SEQUENCE LISTING (258 sequences)

SEQ ID NO: 1 - skipped

SEQ ID NO: 2 - DNA, 786 bp, synthetic construct
  misc_feature 1..786: note = 1H10 VH-VL scFv coding sequence
  source 1..786: mol_type = other DNA
atgctgctgctcgtgaccagcctgctgctgtgcgaactgccccaccctgcctttctgctgatcccccaagtacagcttgttcaaagtggtgctgaagttaaaaagccaggggccagcgttaaggtatcctgcaagggaagtggttacatcttcacatcttacgacatgcactgggtacgacaggctcctggacagggtctggaatggatgggtatcatagacccctcaggaggatctacgagctatgcccaaaaatttcagggaagagtaacaatgaccagggacacgtccatgagcacagtctacatggaactcagcagtctcagatcagaggatacggcggtttactactgtactagggattattcatggagctatttcgactattggggacaaggaaccttggtaacagtgtcttcaggaggcggaggatctggcggagggggctctggaggaggaggatctgcgatacaaatgacgcaaagtcccagcagtttgtccgcctcagtaggcgaccgcgttacgattacgtgtagggcgtctcaagggatcaggatctatctgggctggtatcaacaaaagcctgggaaagccccaaagctccttatatatgcaacatcatccctgcaaagcggcgttccatcccgattcagtggttctggtagcggtacggacttcactctcacaatctcatctcttcaaccagaagactttgcgacgtattactgtttgcaagactacaattatccatggacgttcggccaaggcacgaaagtcgagataaag

SEQ ID NO: 3 - DNA, 783 bp, synthetic construct
  misc_feature 1..783: note = 1A9 VH-VL scFv coding sequence
  source 1..783: mol_type = other DNA
atgctgctgctcgtgaccagcctgctgctgtgcgaactgccccaccctgcctttctgctgatccccgaagtgcagttggttgagtctggaggaggcctggtacagccgggtggtagtcttcggctttcctgtgctgctagcgggtttactttctccatatacgatatgcactgggtgaggcaagcgaccggaaaaggtctggagtgggtctcagcgatcggtacagctggcgatacttactatgcgggcagtgtcaagggacgattcaccataagccgcgaaaacgctaaaaattccctctacttgcaaatgaatagcctgcgagcgggggacaccgccgtatattattgtgctagagagtatagcggatattactttgactattggggtcaaggcactctggtaacggtgtctagcggaggcggaggatctggcggagggggctctggaggaggaggatctgcgattcagatgactcaatccccctcctctctctccgcgtccgtaggggatagggtgacaataacttgtagggcgagccaggacatccgcaatgacctcggctggtatcaacaaaaaccaggcaaggcacctaagatactgatttatggcgcgtcctccttgcaatccggggtgccgtctcggttcagtggttcaggtagtggtacggactttaccttcacaatctctagtctgcaaccggaggatttcgctacttactattgtctccaggagtataattacccctgtacatttgggcaaggcaccaagttggagataaaa

SEQ ID NO: 4 - DNA, 786 bp, synthetic construct
  misc_feature 1..786: note = 1E6 VH-VL scFv coding sequence
  source 1..786: mol_type = other DNA
atgctgctgctcgtgaccagcctgctgctgtgcgaactgccccaccctgcctttctgctgatcccccaagtgcagttggttgaatccggtggcggtgtggttcaaccaggcaggagcttgagactttcatgtgcagcgtccggctttacattctccagctacgacatacattgggtccggcaggcgccaggaaagggcctcgaatgggtcgcggtaatatggtacgacggcagtcataactactacagtgattctgtaaaaggccgctttacgatttcacgcgacaacagcaagaatacactctatttgcaaatgaactctctgcgcgcggaagataccgccgtgtattattgtgcgcgggactacagcgggtcttactacgactactggggccaaggaacccttgtaacggtctctagcggaggcggaggatctggcggagggggctctggaggaggaggatctgcaatacaaatgacgcagtctcctagctcactttctgcaagcgtcggagaccgagttacaattacgtgtcgggcgagccagggaattcggaacgatctcggctggtatcaacagaaacccggcaaagcgccaaaattgcttatatacgcggcatcaaaccttcagagtggtgtgccgtcaagattcagtgggtcaggcagcggaactgactttaccctgactatctctagtctccaacccgaggacttcgcaacgtactattgcctgcaagattactcctacccgcgaacgttcggccaagggacaaaggttgagattaaa

SEQ ID NO: 5 - DNA, 786 bp, synthetic construct
  misc_feature 1..786: note = 1D2 VH-VL scFv coding sequence
  source 1..786: mol_type = other DNA
atgctgctgctcgtgaccagcctgctgctgtgcgaactgccccaccctgcctttctgctgatcccccaggtgcagctggtggaaagcggcggcggcgtggtgcagccgggccgcagcctgcgcctgagctgcgcggcgagcggctttacctttagcagctatgatattcattgggtgcgccaggcgccgggcaaaggcctggaatgggtggcggtgatttggtatgatggcagccagaaatattatgcggatagcgtgaaaggccgctttaccattagccgcgataacagcaaaaacaccctgtatctgcagatgaacagcctgcgcgcggaagataccgcggtgtattattgcgcgcgcgattatagcggcagctattatgattattggggccagggcaccctggtgaccgtgagcagcggaggcggaggatctggcggagggggctctggaggaggaggatctgcgattcagatgacccagagcccgagcagcctgagcgcgagcgtgggcgatcgcgtgaccattacctgccgcgcgagccagggcattcgcaacgatctgggctggtatcagcagaaaccgggcaaagcgccggaactgctgatttatgcgaccagcagcctgcagagcggcgtgccgagccgctttagcggcagcggcagcggcaccgattttaccctgattattagcagcctgcagccggaagattttgcgacctattattgcctgcaggattatagctatccgcgcacctttggccagggcaccaaagtggaaattaaa

SEQ ID NO: 6 - DNA, 786 bp, synthetic construct
  misc_feature 1..786: note = 1B9 VH-VL scFv coding sequence
  source 1..786: mol_type = other DNA
atgctgctgctcgtgaccagcctgctgctgtgcgaactgccccaccctgcctttctgctgatcccccaggtgcagctggtggaaagcggcggcggcgtggtgcagccgggccgcagcctgcgcctgagctgcgcggcgagcggctttatttttagcagctatgatattcattgggtgcgccaggcgccgggcaaaggcctggaatgggtggcggtgatttggtatgatggcagccataactattatagcgatagcgtgaaaggccgctttaccattagccgcgataacagcaaaaacaccctgtatctgcagatgaacagcctgcgcgcggaagataccgcggtgtattattgcgcgcgcgattatagcggcagctattttgattattggggccagggcaccctggtgaccgtgagcagcggaggcggaggatctggcggagggggctctggaggaggaggatctgcgattcagatgacccagagcccgagcagcctgagcgcgagcgtgggcgatcgcgtgaccattacctgccgcgcgagccaggatattcgcaacgatctgggctggtatctgcagcgcccgggcaaagcgccgaaactgctgatttatgcggcgagcagcctgcagagcggcgtgccgagccgctttagcggcagcggcagcggcaccgattttaccctgaccattagcagcctgcagccggaagattttgcgacctattattgcctgcaggattatagctatccgcgcacctttggccagggcaccaccgtggaaattaaa

SEQ ID NO: 7 - DNA, 792 bp, synthetic construct
  misc_feature 1..792: note = 1H8 VH-VL scFv coding sequence
  source 1..792: mol_type = other DNA
atgctgctgctcgtgaccagcctgctgctgtgcgaactgccccaccctgcctttctgctgatcccccaggttcagttggttgaatcaggaggtggtgttgttcagcctggtgggagtctccgcctttcctgtgcggcatctggcttcaccttcggtagttatgggatgcattgggtacgccaagcgcctggcaaaggtctggaatgggtggccgtaatatggtacgatggatctaatgagtactacgcagactccgtgaaagggagattcactgtatcaagagataattctaagcacacgttgtatcttcagatgaacagactccgggcagaggacacagcagtttactactgtgcgcgggacctcgattatgactctagcgggggtgattactggggccaagggattttggttctcgtaagctctggaggcggaggatctggcggagggggctctggaggaggaggatctgaaatagttttgacccaaagccccgactttcagtccgtaacccctaaagaaaaggtcactattacctgtagggcttcccaaaacataggaggcaaccttcactggtatcagcagaagccggaccagtcccctaaattgttgattaggtatgcgacacagcctttttcaggcgtcccctcaagattcggaggctctggttctgggactgacttcaccctgactatcaatagtctcgaggcagaagacgcggccacttattactgccaccaaagtagctccctcccactcactttcggtggtggcaccaaagtagagataaag

SEQ ID NO: 8 - DNA, 792 bp, synthetic construct
  misc_feature 1..792: note = 2D3 VH-VL scFv coding sequence
  source 1..792: mol_type = other DNA
atgctgctgctcgtgaccagcctgctgctgtgcgaactgccccaccctgcctttctgctgatccccgaggtgcaattgctggaaagtggaggaggactcgtgcagcccggaggttcccttagcctttcttgcgctgcaagtgggtttacgttctctatatatgccatgtcttgggtgcggcaagcccccggaaaaggattggaatgggtatctgccattagtgattctgggggtacgacctattatgcagatagtgtaaaagggagattcactatctcacgcgacaattcaaagaatatgctttaccttgagatgaacagtcttcgagcagaggatacagccatatactattgcgctaaacgcacccgctacttcaacggaatggatgtatggggacagggtacaacagttactgtttctagcggaggcggaggatctggcggagggggctctggaggaggaggatctgagattgtaatgacgcagtctccagcgacgctttctcttagtccgggagaaagagccacactgtcctgccgggcgtcccaatccggttctagctcctttctgtcatggtatcaacagaagccaggtcaggcacctcgccttcttatttacggtgcatccactcgcgcgaccgggattcctgcaagattttccgggtctgggtctggcacagatttcacgttgactatcagtagtctgcagccagaggatttcgcagtctattactgtcaacaagactacaatcttcctttcacgtttggtcccggaactaaggttgatataaaa

SEQ ID NO: 9 - DNA, 801 bp, synthetic construct
  misc_feature 1..801: note = 2E3 VH-VL scFv coding sequence
  source 1..801: mol_type = other DNA
atgctgctgctcgtgaccagcctgctgctgtgcgaactgccccaccctgcctttctgctgatcccccaagtgtgtctggtcgagagtggtggaggcgtggttcaaccagggaagagcctccggctctcctgtgcagccagtggatttaccttttcatcctacggtatgcactgggtccgccaggcacccggaaaaggacttgaatgggtggctgtaatttggtatggtggctccaacaaatactatgcagatagtgtaaaaggtcgctttactatcagtcgggacaatagcaaaaataccctctacttgcagatgaatagcttgcgggccgaggataccgctgtctactactgtgcaagagatggtaccggcgaaaactattactactacgtaatggatgtctggggacagggcacgaccgttacagtctcatccggaggcggaggatctggcggagggggctctggaggaggaggatctgagattgtattgactcaaagtccaggtaccctctcccttagccccggagaaagagctacgctcagctgccgcgcttcacagtctgtatcatcctcctatctcgcttggtatcagcagaagcctggtcaagctcctcgccttttgatctatggtacatccagccgggccacaggcatcccggatcggttttccggtagcggatctggtacggattttactcttacaatttcccgactcgaaccagaagactttgcggtatattattgtcagcaatacggttcttcaccgacctttgggggaggcacaaaagtcgagatcaaa

SEQ ID NO: 10 - DNA, 36 bp, synthetic construct
  misc_feature 1..36: note = IgG4 hinge coding sequence-A
  source 1..36: mol_type = other DNA
gagtctaagtacggaccgccctgccccccttgccct

SEQ ID NO: 11 - DNA, 36 bp, synthetic construct
  misc_feature 1..36: note = IgG4 hinge coding sequence-B
  source 1..36: mol_type = other DNA
gagtctaagtacggaccgccttgcccaccgtgccca

SEQ ID NO: 12 - DNA, 321 bp, synthetic construct
  misc_feature 1..321: note = IgG4-int(DS) coding sequence
  source 1..321: mol_type = other DNA
ggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaag

SEQ ID NO: 13 - DNA, 648 bp, synthetic construct
  misc_feature 1..648: note = IgG4-long coding sequence
  source 1..648: mol_type = other DNA
gcaccacctgtggcaggaccgtcagtcttcctcttcccaccaaaacccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccaaagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaag

SEQ ID NO: 14 - DNA, 336 bp, synthetic construct
  misc_feature 1..336: note = CD3z coding sequence
  source 1..336: mol_type = other DNA
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 15 - protein, 112 aa, synthetic construct
  REGION 1..112: note = CD3z protein-A
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 114 aa, synthetic construct
  REGION 1..114: note = CD3z protein-B
ELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 126 bp, synthetic construct
  misc_feature 1..126: note = 4-1BB signaling coding sequence-A
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 18 - DNA, 129 bp, synthetic construct
  misc_feature 1..129: note = 4-1BB signaling coding sequence-B
  source 1..129: mol_type = other DNA
gtgaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 19 - protein, 43 aa, synthetic construct
  REGION 1..43: note = 4-1BB protein-a
VKRGRKKLLYIFKQPFMRPVQTTQEEDGCSCRFPEEEEGGCEL

SEQ ID NO: 20 - protein, 41 aa, synthetic construct
  REGION 1..41: note = 4-1BB protein-B
VKRGRKKLLYIFKQPFMRPVQTTQEEDGCSCRFPEEEEGGC

SEQ ID NO: 21 - DNA, 84 bp, synthetic construct
  misc_feature 1..84: note = CD28TM coding sequence-A
  source 1..84: mol_type = other DNA
atgttctgggtgctggtggtggtcggaggcgtgctggcctgctacagcctgctggtcaccgtggccttcatcatcttttgggtg

SEQ ID NO: 22 - DNA, 81 bp, synthetic construct
  misc_feature 1..81: note = CD28TM coding sequence-B
  source 1..81: mol_type = other DNA
atgttctgggtgctggtggtggtcggaggcgtgctggcctgctacagcctgctggtcaccgtggccttcatcatcttttgg

SEQ ID NO: 23 - DNA, 81 bp, synthetic construct
  misc_feature 1..81: note = CD28TM coding sequence-C
  source 1..81: mol_type = other DNA
atgttctgggtgctggtggtggtgggcggggtgctggcctgctacagcctgctggtgacagtggccttcatcatcttttgg

SEQ ID NO: 24 - protein, 27 aa, synthetic construct
  REGION 1..27: note = CD28TM protein-A
MFWVLVVVGGVLACYSLLVTVAFIIFW

SEQ ID NO: 25 - protein, 26 aa, synthetic construct
  REGION 1..26: note = CD28TM protein-B
FWVLVVVGGVLACYSLLVTVAFIIFW

SEQ ID NO: 26 - DNA, 972 bp, synthetic construct
  misc_feature 1..972: note = tCD19 coding sequence
  source 1..972: mol_type = other DNA
atgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 27 - DNA, 72 bp, synthetic construct
  misc_feature 1..72: note = T2A coding sequence
  source 1..72: mol_type = other DNA
ctcgagggcggcggagagggcagaggaagtcttctaacatgcggtgacgtggaggagaatccaggccctagg

SEQ ID NO: 28 - protein, 21 aa, synthetic construct
  REGION 1..21: note = Thoseaasigna Virus 2A (T2A) Peptide
  REGION 1..3: note = MISC_FEATURE - (GlySerGly) residues can be added to the 5' end of the peptide to improve cleavage efficiency
GSGEGRGSLLTCGDVEENPGP

SEQ ID NO: 29 - protein, 22 aa, synthetic construct
  REGION 1..22: note = Porcine Teschovirus-1 2a (P2A) Peptide
  REGION 1..3: note = MISC_FEATURE - (GlySerGly) residues can be added to the 5' end of the peptide to improve cleavage efficiency
GSGATNFSLLKQAGDVEENPGP

SEQ ID NO: 30 - protein, 23 aa, synthetic construct
  REGION 1..23: note = Equine Rhinitis A Virus (E2A) Peptide
  REGION 1..3: note = MISC_FEATURE - (GlySerGly) residues can be added to the 5' end of the peptide to improve cleavage efficiency
GSGQCTNYALLKLAGDVESNPGP

SEQ ID NO: 31 - protein, 25 aa, synthetic construct
  REGION 1..25: note = Foot-And-Mouth Disease Virus 2A (F2A) Peptide
  REGION 1..3: note = MISC_FEATURE - (GlySerGly) residues can be added to the 5' end of the peptide to improve cleavage efficiency
GSGVKQTLNFDLLKLAGDVESNPGP

SEQ ID NO: 32 - DNA, 544 bp, synthetic construct
  misc_feature 1..544: note = EF1 promoter-A
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 33 - DNA, 545 bp, synthetic construct
  misc_feature 1..545: note = EF1 promoter-B
  source 1..545: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacg

SEQ ID NO: 34 - DNA, 113 bp, synthetic construct
  misc_feature 1..113: note = Psi
  source 1..113: mol_type = other DNA
ctcggcttgctgaagcgcgcacggcaagaggcgaggggcggcgactggtgagtacgccaaaaattttgactagcggaggctagaaggagagagatgggtgcgagagcgtcagt

SEQ ID NO: 35 - DNA, 333 bp, synthetic construct
  misc_feature 1..333: note = RRE
  source 1..333: mol_type = other DNA
gcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatc

SEQ ID NO: 36 - DNA, 179 bp, synthetic construct
  misc_feature 1..179: note = Flap
  source 1..179: mol_type = other DNA
tacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgg

SEQ ID NO: 37 - DNA, 66 bp, synthetic construct
  misc_feature 1..66: note = GM-CSFR encoding sequence
  source 1..66: mol_type = other DNA
atgctgctgctcgtgaccagcctgctgctgtgcgaactgccccaccctgcctttctgctgatcccc

SEQ ID NO: 38 - DNA, 601 bp, synthetic construct
  misc_feature 1..601: note = WPRE
  source 1..601: mol_type = other DNA
atcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgat

SEQ ID NO: 39 - DNA, 105 bp, synthetic construct
  misc_feature 1..105: note = delU3
  source 1..105: mol_type = other DNA
gtacctttaagaccaatgacttacaaggcagctgtagatcttagccactttttaaaagaaaaggggggactggaagggctaattcactcccaaagaagacaagat

SEQ ID NO: 40 - DNA, 81 bp, synthetic construct
  misc_feature 1..81: note = R
  source 1..81: mol_type = other DNA
ctgctttttgcctgtactgggtctctctggttagaccagatctgagcctgggagctctctggctaactagggaacccactg

SEQ ID NO: 41 - DNA, 114 bp, synthetic construct
  misc_feature 1..114: note = U5
  source 1..114: mol_type = other DNA
cttaagcctcaataaagcttgccttgagtgcttcaagtagtgtgtgcccgtctgttgtgtgactctggtaactagagatccctcagacccttttagtcagtgtggaaaatctct

SEQ ID NO: 42 - DNA, 859 bp, synthetic construct
  misc_feature 1..859: note = AmpR
  source 1..859: mol_type = other DNA
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggt

SEQ ID NO: 43 - DNA, 882 bp, synthetic construct
  misc_feature 1..882: note = CoE1 origin
  source 1..882: mol_type = other DNA
aaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagag

SEQ ID NO: 44 - DNA, 200 bp, synthetic construct
  misc_feature 1..200: note = SV40
  source 1..200: mol_type = other DNA
ggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 45 - DNA, 600 bp, synthetic construct
  misc_feature 1..600: note = CMV
  source 1..600: mol_type = other DNA
atcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattc

SEQ ID NO: 46 - DNA, 3288 bp, synthetic construct
  misc_feature 1..3288: note = 1H10-intDS-41bb-3z-T-CD19t Top Strand
  source 1..3288: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagccaccatgctgctgctcgtgaccagcctgctgctgtgcgaactgccccaccctgcctttctgctgatcccccaagtacagcttgttcaaagtggtgctgaagttaaaaagccaggggccagcgttaaggtatcctgcaagggaagtggttacatcttcacatcttacgacatgcactgggtacgacaggctcctggacagggtctggaatggatgggtatcatagacccctcaggaggatctacgagctatgcccaaaaatttcagggaagagtaacaatgaccagggacacgtccatgagcacagtctacatggaactcagcagtctcagatcagaggatacggcggtttactactgtactagggattattcatggagctatttcgactattggggacaaggaaccttggtaacagtgtcttcaggaggcggaggatctggcggagggggctctggaggaggaggatctgcgatacaaatgacgcaaagtcccagcagtttgtccgcctcagtaggcgaccgcgttacgattacgtgtagggcgtctcaagggatcaggatctatctgggctggtatcaacaaaagcctgggaaagccccaaagctccttatatatgcaacatcatccctgcaaagcggcgttccatcccgattcagtggttctggtagcggtacggacttcactctcacaatctcatctcttcaaccagaagactttgcgacgtattactgtttgcaagactacaattatccatggacgttcggccaaggcacgaaagtcgagataaaggagtctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatccaggccctaggatgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 47 - DNA, 2967 bp, synthetic construct
  misc_feature 1..2967: note = 1H10-sh-41bb-3z-T-CD19t Top Strand
  source 1..2967: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagccaccatgctgctgctcgtgaccagcctgctgctgtgcgaactgccccaccctgcctttctgctgatcccccaagtacagcttgttcaaagtggtgctgaagttaaaaagccaggggccagcgttaaggtatcctgcaagggaagtggttacatcttcacatcttacgacatgcactgggtacgacaggctcctggacagggtctggaatggatgggtatcatagacccctcaggaggatctacgagctatgcccaaaaatttcagggaagagtaacaatgaccagggacacgtccatgagcacagtctacatggaactcagcagtctcagatcagaggatacggcggtttactactgtactagggattattcatggagctatttcgactattggggacaaggaaccttggtaacagtgtcttcaggaggcggaggatctggcggagggggctctggaggaggaggatctgcgatacaaatgacgcaaagtcccagcagtttgtccgcctcagtaggcgaccgcgttacgattacgtgtagggcgtctcaagggatcaggatctatctgggctggtatcaacaaaagcctgggaaagccccaaagctccttatatatgcaacatcatccctgcaaagcggcgttccatcccgattcagtggttctggtagcggtacggacttcactctcacaatctcatctcttcaaccagaagactttgcgacgtattactgtttgcaagactacaattatccatggacgttcggccaaggcacgaaagtcgagataaaggagtctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatccaggccctaggatgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 48 - DNA, 3285 bp, synthetic construct
  misc_feature 1..3285: note = 1A9-intDS-41bb-3z-T-CD19t Top Strand
  source 1..3285: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagccaccatgctgctgctcgtgaccagcctgctgctgtgcgaactgccccaccctgcctttctgctgatccccgaagtgcagttggttgagtctggaggaggcctggtacagccgggtggtagtcttcggctttcctgtgctgctagcgggtttactttctccatatacgatatgcactgggtgaggcaagcgaccggaaaaggtctggagtgggtctcagcgatcggtacagctggcgatacttactatgcgggcagtgtcaagggacgattcaccataagccgcgaaaacgctaaaaattccctctacttgcaaatgaatagcctgcgagcgggggacaccgccgtatattattgtgctagagagtatagcggatattactttgactattggggtcaaggcactctggtaacggtgtctagcggaggcggaggatctggcggagggggctctggaggaggaggatctgcgattcagatgactcaatccccctcctctctctccgcgtccgtaggggatagggtgacaataacttgtagggcgagccaggacatccgcaatgacctcggctggtatcaacaaaaaccaggcaaggcacctaagatactgatttatggcgcgtcctccttgcaatccggggtgccgtctcggttcagtggttcaggtagtggtacggactttaccttcacaatctctagtctgcaaccggaggatttcgctacttactattgtctccaggagtataattacccctgtacatttgggcaaggcaccaagttggagataaaagagtctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatccaggccctaggatgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 49 - DNA, 2964 bp, synthetic construct
  misc_feature 1..2964: note = 1A9-sh-41bb-3z-T-CD19t Top Strand
  source 1..2964: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagccaccatgctgctgctcgtgaccagcctgctgctgtgcgaactgccccaccctgcctttctgctgatccccgaagtgcagttggttgagtctggaggaggcctggtacagccgggtggtagtcttcggctttcctgtgctgctagcgggtttactttctccatatacgatatgcactgggtgaggcaagcgaccggaaaaggtctggagtgggtctcagcgatcggtacagctggcgatacttactatgcgggcagtgtcaagggacgattcaccataagccgcgaaaacgctaaaaattccctctacttgcaaatgaatagcctgcgagcgggggacaccgccgtatattattgtgctagagagtatagcggatattactttgactattggggtcaaggcactctggtaacggtgtctagcggaggcggaggatctggcggagggggctctggaggaggaggatctgcgattcagatgactcaatccccctcctctctctccgcgtccgtaggggatagggtgacaataacttgtagggcgagccaggacatccgcaatgacctcggctggtatcaacaaaaaccaggcaaggcacctaagatactgatttatggcgcgtcctccttgcaatccggggtgccgtctcggttcagtggttcaggtagtggtacggactttaccttcacaatctctagtctgcaaccggaggatttcgctacttactattgtctccaggagtataattacccctgtacatttgggcaaggcaccaagttggagataaaagagtctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatccaggccctaggatgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 50 - DNA, 3288 bp, synthetic construct
  misc_feature 1..3288: note = 1E6-intDS-41bb-3z-T-CD19t Top Strand
  source 1..3288: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagccaccatgctgctgctcgtgaccagcctgctgctgtgcgaactgccccaccctgcctttctgctgatcccccaagtgcagttggttgaatccggtggcggtgtggttcaaccaggcaggagcttgagactttcatgtgcagcgtccggctttacattctccagctacgacatacattgggtccggcaggcgccaggaaagggcctcgaatgggtcgcggtaatatggtacgacggcagtcataactactacagtgattctgtaaaaggccgctttacgatttcacgcgacaacagcaagaatacactctatttgcaaatgaactctctgcgcgcggaagataccgccgtgtattattgtgcgcgggactacagcgggtcttactacgactactggggccaaggaacccttgtaacggtctctagcggaggcggaggatctggcggagggggctctggaggaggaggatctgcaatacaaatgacgcagtctcctagctcactttctgcaagcgtcggagaccgagttacaattacgtgtcgggcgagccagggaattcggaacgatctcggctggtatcaacagaaacccggcaaagcgccaaaattgcttatatacgcggcatcaaaccttcagagtggtgtgccgtcaagattcagtgggtcaggcagcggaactgactttaccctgactatctctagtctccaacccgaggacttcgcaacgtactattgcctgcaagattactcctacccgcgaacgttcggccaagggacaaaggttgagattaaagagtctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatccaggccctaggatgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 51 - DNA, 2967 bp, synthetic construct
  misc_feature 1..2967: note = 1E6-sh-41bb-3z-T-CD19t Top Strand
  source 1..2967: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagccaccatgctgctgctcgtgaccagcctgctgctgtgcgaactgccccaccctgcctttctgctgatcccccaagtgcagttggttgaatccggtggcggtgtggttcaaccaggcaggagcttgagactttcatgtgcagcgtccggctttacattctccagctacgacatacattgggtccggcaggcgccaggaaagggcctcgaatgggtcgcggtaatatggtacgacggcagtcataactactacagtgattctgtaaaaggccgctttacgatttcacgcgacaacagcaagaatacactctatttgcaaatgaactctctgcgcgcggaagataccgccgtgtattattgtgcgcgggactacagcgggtcttactacgactactggggccaaggaacccttgtaacggtctctagcggaggcggaggatctggcggagggggctctggaggaggaggatctgcaatacaaatgacgcagtctcctagctcactttctgcaagcgtcggagaccgagttacaattacgtgtcgggcgagccagggaattcggaacgatctcggctggtatcaacagaaacccggcaaagcgccaaaattgcttatatacgcggcatcaaaccttcagagtggtgtgccgtcaagattcagtgggtcaggcagcggaactgactttaccctgactatctctagtctccaacccgaggacttcgcaacgtactattgcctgcaagattactcctacccgcgaacgttcggccaagggacaaaggttgagattaaagagtctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatccaggccctaggatgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 52 - protein, 6 aa, synthetic construct
  REGION 1..6: note = First six amino acids of the CH2 domain in IgG4
APEFLG

SEQ ID NO: 53 - protein, 5 aa, synthetic construct
  REGION 1..5: note = First five amino acids of IgG2
APPVA

SEQ ID NO: 54 - protein, 11 aa, synthetic construct
  REGION 1..11: note = CDRL1 of 1H10
RASQGIRIYLG

SEQ ID NO: 55 - protein, 8 aa, synthetic construct
  REGION 1..8: note = CDRL2 of 1H10 and 1D2
YATSSLQS

SEQ ID NO: 56 - protein, 9 aa, synthetic construct
  REGION 1..9: note = CDRL3 of 1H10
LQDYNYPWT

SEQ ID NO: 57 - protein, 13 aa, synthetic construct
  REGION 1..13: note = CDRH1 of 1H10
KGSGYIFTSYDMH

SEQ ID NO: 58 - protein, 10 aa, synthetic construct
  REGION 1..10: note = CDRH2 of 1H10
IIDPSGGSTS

SEQ ID NO: 59 - protein, 11 aa, synthetic construct
  REGION 1..11: note = CDRH3 of 1H10
TRDYSWSYFDY

SEQ ID NO: 60 - protein, 11 aa, synthetic construct
  REGION 1..11: note = CDRL1 of 1A9 and 1B9
RASQDIRNDLG

SEQ ID NO: 61 - protein, 8 aa, synthetic construct
  REGION 1..8: note = CDRL2 of 1A9
YGASSLQS

SEQ ID NO: 62 - protein, 9 aa, synthetic construct
  REGION 1..9: note = CDRL3 of 1A9
LQEYNYPCT

SEQ ID NO: 63 - protein, 13 aa, synthetic construct
  REGION 1..13: note = CDRH1 of 1A9
AASGFTFSIYDMH

SEQ ID NO: 64 - protein, 9 aa, synthetic construct
  REGION 1..9: note = CDRH2 of 1A9
AIGTAGDTY

SEQ ID NO: 65 - protein, 11 aa, synthetic construct
  REGION 1..11: note = CDRH3 of 1A9
AREYSGYYFDY

SEQ ID NO: 66 - protein, 11 aa, synthetic construct
  REGION 1..11: note = CDRL1 of 1E6 and 1D2
RASQGIRNDLG

SEQ ID NO: 67 - protein, 8 aa, synthetic construct
  REGION 1..8: note = CDRL2 of 1E6
YAASNLQS

SEQ ID NO: 68 - protein, 9 aa, synthetic construct
  REGION 1..9: note = CDRL3 of 1E6, 1D2, and 1B9
LQDYSYPRT

SEQ ID NO: 69 - protein, 13 aa, synthetic construct
  REGION 1..13: note = CDRH1 of 1E6 and 1D2
AASGFTFSSYDIH

SEQ ID NO: 70 - protein, 10 aa, synthetic construct
  REGION 1..10: note = CDRH2 of 1E6 and 1B9
VIWYDGSHNY

SEQ ID NO: 71 - protein, 11 aa, synthetic construct
  REGION 1..11: note = CDRH3 of 1E6 and 1D2
ARDYSGSYYDY

SEQ ID NO: 72 - protein, 10 aa, synthetic construct
  REGION 1..10: note = CDRH2 of 1D2
VIWYDGSQKY

SEQ ID NO: 73 - protein, 8 aa, synthetic construct
  REGION 1..8: note = CDRL2 of 1B9
YAASSLQS

SEQ ID NO: 74 - protein, 13 aa, synthetic construct
  REGION 1..13: note = CDRH1 of 1B9
AASGFIFSSYDIH

SEQ ID NO: 75 - protein, 11 aa, synthetic construct
  REGION 1..11: note = CDRH3 of 1B9
ARDYSGSYFDY

SEQ ID NO: 76 - protein, 11 aa, synthetic construct
  REGION 1..11: note = CDRL1 of 1H8
RASQNIGGNLH

SEQ ID NO: 77 - protein, 7 aa, synthetic construct
  REGION 1..7: note = CDRL2 of 1H8
YATQPFS

SEQ ID NO: 78 - protein, 9 aa, synthetic construct
  REGION 1..9: note = CDRL3 of 1H8
HQSSSLPLT

SEQ ID NO: 79 - protein, 5 aa, synthetic construct
  REGION 1..5: note = CDRH1 of 1H8 and 2E3
SYGMH

SEQ ID NO: 80 - protein, 16 aa, synthetic construct
  REGION 1..16: note = CDRH2 of 1H8
IWYDGSNEYYADSVKG

SEQ ID NO: 81 - protein, 8 aa, synthetic construct
  REGION 1..8: note = CDRH3 of 1H8
DLDYDSSG

SEQ ID NO: 82 - protein, 9 aa, synthetic construct
  REGION 1..9: note = CDRL1 of 2D3
QSGSSSFLS

SEQ ID NO: 83 - protein, 7 aa, synthetic construct
  REGION 1..7: note = CDRL2 of 2D3
GASTRAT

SEQ ID NO: 84 - protein, 9 aa, synthetic construct
  REGION 1..9: note = CDRL3 of 2D3
QQDYNLPFT

SEQ ID NO: 85 - protein, 5 aa, synthetic construct
  REGION 1..5: note = CDRH1 of 2D3
IYAMS

SEQ ID NO: 86 - protein, 16 aa, synthetic construct
  REGION 1..16: note = CDRH2 of 2D3
ISDSGGTTYYADSVKG

SEQ ID NO: 87 - protein, 7 aa, synthetic construct
  REGION 1..7: note = CDRH3 of 2D3
RTRYFNG

SEQ ID NO: 88 - protein, 9 aa, synthetic construct
  REGION 1..9: note = CDRL1 of 2E3
QSVSSSYLA

SEQ ID NO: 89 - protein, 7 aa, synthetic construct
  REGION 1..7: note = CDRL2 of 2E3
GTSSRAT

SEQ ID NO: 90 - protein, 8 aa, synthetic construct
  REGION 1..8: note = CDRL3 of 2E3
QQYGSSPT

SEQ ID NO: 91 - protein, 16 aa, synthetic construct
  REGION 1..16: note = CDRH2 of 2E3
IWYGGSNKYYADSVKG

SEQ ID NO: 92 - protein, 11 aa, synthetic construct
  REGION 1..11: note = CDRH3 of 2E3
DGTGENYYYYV

SEQ ID NO: 93 - protein, 8 aa, synthetic construct
  REGION 1..8: note = CDRL2 of 1H8
RYATQPFS

SEQ ID NO: 94 - protein, 13 aa, synthetic construct
  REGION 1..13: note = CDRH1 of 1H8
AASGFTFGSYGMH

SEQ ID NO: 95 - protein, 10 aa, synthetic construct
  REGION 1..10: note = CDRH2 of 1H8
VIWYDGSNEY

SEQ ID NO: 96 - protein, 13 aa, synthetic construct
  REGION 1..13: note = CDRH3 of 1H8
ARDLDYDSSGGDY

SEQ ID NO: 97 - protein, 12 aa, synthetic construct
  REGION 1..12: note = CDRL1 of 2D3
RASQSGSSSFLS

SEQ ID NO: 98 - protein, 8 aa, synthetic construct
  REGION 1..8: note = CDRL2 of 2D3
YGASTRAT

SEQ ID NO: 99 - protein, 13 aa, synthetic construct
  REGION 1..13: note = CDRH1 of 2D3
AASGFTFSIYAMS

SEQ ID NO: 100 - protein, 10 aa, synthetic construct
  REGION 1..10: note = CDRH2 of 2D3
AISDSGGTTY

SEQ ID NO: 101 - protein, 12 aa, synthetic construct
  REGION 1..12: note = CDRH3 of 2D3
AKRTRYFNGMDV

SEQ ID NO: 102 - protein, 12 aa, synthetic construct
  REGION 1..12: note = CDRL1 of 2E3
RASQSVSSSYLA

SEQ ID NO: 103 - protein, 8 aa, synthetic construct
  REGION 1..8: note = CDRL2 of 2E3
YGTSSRAT

SEQ ID NO: 104 - protein, 13 aa, synthetic construct
  REGION 1..13: note = CDRH1 of 2E3
AASGFTFSSYGMH

SEQ ID NO: 105 - protein, 10 aa, synthetic construct
  REGION 1..10: note = CDRH2 of 2E3
VIWYGGSNKY

SEQ ID NO: 106 - protein, 16 aa, synthetic construct
  REGION 1..16: note = CDRH3 of 2E3
ARDGTGENYYYYVMDV

SEQ ID NO: 107 - protein, 6 aa, synthetic construct
  REGION 1..6: note = CDRL1 of 1H10
QGIRIY

SEQ ID NO: 108 - protein, 8 aa, synthetic construct
  REGION 1..8: note = CDRH1 of 1H10
GYIFTSYD

SEQ ID NO: 109 - protein, 8 aa, synthetic construct
  REGION 1..8: note = CDRH2 of 1H10
IDPSGGST

SEQ ID NO: 110 - protein, 6 aa, synthetic construct
  REGION 1..6: note = CDRL1 of 1A9 and 1B9
QDIRND

SEQ ID NO: 111 - protein, 8 aa, synthetic construct
  REGION 1..8: note = CDRH1 of 1A9
GFTFSIYD

SEQ ID NO: 112 - protein, 7 aa, synthetic construct
  REGION 1..7: note = CDRH2 of 1A9
IGTAGDT

SEQ ID NO: 113 - protein, 6 aa, synthetic construct
  REGION 1..6: note = CDRL1 of 1E6 and 1D2
QGIRND

SEQ ID NO: 114 - protein, 8 aa, synthetic construct
  REGION 1..8: note = CDRH1 of 1E6 and 1D2
GFTFSSYD

SEQ ID NO: 115 - protein, 8 aa, synthetic construct
  REGION 1..8: note = CDRH2 of 1E6 and 1B9
IWYDGSHN

SEQ ID NO: 116 - protein, 8 aa, synthetic construct
  REGION 1..8: note = CDRH2 of 1D2
IWYDGSQK

SEQ ID NO: 117 - protein, 8 aa, synthetic construct
  REGION 1..8: note = CDRH1 of 1B9
GFIFSSYD

SEQ ID NO: 118 - protein, 6 aa, synthetic construct
  REGION 1..6: note = CDRL1 of 1H8
QNIGGN

SEQ ID NO: 119 - protein, 8 aa, synthetic construct
  REGION 1..8: note = CDRH1 of 1H8
GFTFGSYG

SEQ ID NO: 120 - protein, 8 aa, synthetic construct
  REGION 1..8: note = CDRH2 of 1H8
IWYDGSNE

SEQ ID NO: 121 - protein, 7 aa, synthetic construct
  REGION 1..7: note = CDRL1 of 2D3
QSGSSSF

SEQ ID NO: 122 - protein, 8 aa, synthetic construct
  REGION 1..8: note = CDRH1 of 2D3
GFTFSIYA

SEQ ID NO: 123 - protein, 8 aa, synthetic construct
  REGION 1..8: note = CDRH2 of 2D3
ISDSGGTT

SEQ ID NO: 124 - protein, 7 aa, synthetic construct
  REGION 1..7: note = CDRL1 of 2E3
QSVSSSY

SEQ ID NO: 125 - protein, 8 aa, synthetic construct
  REGION 1..8: note = CDRH1 of 2E3
GFTFSSYG

SEQ ID NO: 126 - protein, 8 aa, synthetic construct
  REGION 1..8: note = CDRH2 of 2E3
IWYGGSNK

SEQ ID NO: 127 - protein, 7 aa, synthetic construct
  REGION 1..7: note = CDRL2 of 1H10 and 1D2
ATSSLQS

SEQ ID NO: 128 - protein, 5 aa, synthetic construct
  REGION 1..5: note = CDRH1 of 1H10
SYDMH

SEQ ID NO: 129 - protein, 17 aa, synthetic construct
  REGION 1..17: note = CDRH2 of 1H10
IIDPSGGSTSYAQKFQG

SEQ ID NO: 130 - protein, 9 aa, synthetic construct
  REGION 1..9: note = CDRH3 of 1H10
DYSWSYFDY

SEQ ID NO: 131 - protein, 7 aa, synthetic construct
  REGION 1..7: note = CDRL2 of 1A9
GASSLQS

SEQ ID NO: 132 - protein, 5 aa, synthetic construct
  REGION 1..5: note = CDRH1 of 1A9
IYDMH

SEQ ID NO: 133 - protein, 16 aa, synthetic construct
  REGION 1..16: note = CDRH2 of 1A9
AIGTAGDTYYAGSVKG

SEQ ID NO: 134 - protein, 9 aa, synthetic construct
  REGION 1..9: note = CDRH3 of 1A9
EYSGYYFDY

SEQ ID NO: 135 - protein, 7 aa, synthetic construct
  REGION 1..7: note = CDRL2 of 1E6
AASNLQS

SEQ ID NO: 136 - protein, 5 aa, synthetic construct
  REGION 1..5: note = CDRH1 of 1E6, 1D2, and 1B9
SYDIH

SEQ ID NO: 137 - protein, 17 aa, synthetic construct
  REGION 1..17: note = CDRH2 of 1E6 and 1B9
VIWYDGSHNYYSDSVKG

SEQ ID NO: 138 - protein, 9 aa, synthetic construct
  REGION 1..9: note = CDRH3 of 1E6 and 1D2
DYSGSYYDY

SEQ ID NO: 139 - protein, 17 aa, synthetic construct
  REGION 1..17: note = CDRH2 of 1D2
VIWYDGSQKYYADSVKG

SEQ ID NO: 140 - protein, 7 aa, synthetic construct
  REGION 1..7: note = CDRL2 of 1B9
AASSLQS

SEQ ID NO: 141 - protein, 9 aa, synthetic construct
  REGION 1..9: note = CDRH3 of 1B9
DYSGSYFDY

SEQ ID NO: 142 - protein, 17 aa, synthetic construct
  REGION 1..17: note = CDRH2 of 1H8
VIWYDGSNEYYADSVKG

SEQ ID NO: 143 - protein, 11 aa, synthetic construct
  REGION 1..11: note = CDRH3 of 1H8
DLDYDSSGGDY

SEQ ID NO: 144 - protein, 17 aa, synthetic construct
  REGION 1..17: note = CDRH2 of 2D3
AISDSGGTTYYADSVKG

SEQ ID NO: 145 - protein, 10 aa, synthetic construct
  REGION 1..10: note = CDRH3 of 2D3
RTRYFNGMDV

SEQ ID NO: 146 - protein, 17 aa, synthetic construct
  REGION 1..17: note = CDRH2 of 2E3
VIWYGGSNKYYADSVKG

SEQ ID NO: 147 - protein, 14 aa, synthetic construct
  REGION 1..14: note = CDRH3 of 2E3
DGTGENYYYYVMDV

SEQ ID NO: 148 - protein, 7 aa, synthetic construct
  REGION 1..7: note = CDRH1 of 1H10
GYIFTSY

SEQ ID NO: 149 - protein, 6 aa, synthetic construct
  REGION 1..6: note = CDRH2 of 1H10
DPSGGS

SEQ ID NO: 150 - protein, 7 aa, synthetic construct
  REGION 1..7: note = CDRH1 of 1A9 and 2D3
GFTFSIY

SEQ ID NO: 151 - protein, 5 aa, synthetic construct
  REGION 1..5: note = CDRH2 of 1A9
GTAGD

SEQ ID NO: 152 - protein, 7 aa, synthetic construct
  REGION 1..7: note = CDRH1 of 1E6, 1D2, and 2E3
GFTFSSY

SEQ ID NO: 153 - protein, 6 aa, synthetic construct
  REGION 1..6: note = CDRH2 of 1E6 and 1B9
WYDGSH

SEQ ID NO: 154 - protein, 6 aa, synthetic construct
  REGION 1..6: note = CDRH2 of 1D2
WYDGSQ

SEQ ID NO: 155 - protein, 7 aa, synthetic construct
  REGION 1..7: note = CDRH1 of 1B9
GFIFSSY

SEQ ID NO: 156 - protein, 7 aa, synthetic construct
  REGION 1..7: note = CDRH1 of 1H8
GFTFGSY

SEQ ID NO: 157 - protein, 6 aa, synthetic construct
  REGION 1..6: note = CDRH2 of 1H8
WYDGSN

SEQ ID NO: 158 - protein, 20 aa, synthetic construct
  REGION 1..20: note = IgK signal peptide
METDTLLLWVLLLWVPGSTG

SEQ ID NO: 159 - protein, 6 aa, synthetic construct
  REGION 1..6: note = CDRH2 of 2E3
WYGGSN

SEQ ID NO: 160 - protein, 5 aa, synthetic construct
  REGION 1..5: note = GlySer linker
  REGION 1..5: note = MISC_FEATURE - Entire sequence can be repeated n times wherein n is an integer including 1, 2, 3, 4, 5, 6, 7, 8, 9, or more
GGGGS

SEQ ID NO: 161 - protein, 90 aa, synthetic construct
  REGION 1..90: note = GlySer linker
  REGION 5..40: note = MISC_FEATURE - Residues can be absent or present
  REGION 45..90: note = MISC_FEATURE - Residues can be absent or present
GGGSGGGSGGGSGGGSGGGSGGGSGGGSGGGSGGGSGGGSGGGGSGGGGSGGGGSGGGGSGGGGSGGGGSGGGGSGGGGSGGGGSGGGGS

SEQ ID NO: 162 - protein, 70 aa, synthetic construct
  REGION 1..70: note = GlySer linker
  REGION 5..40: note = MISC_FEATURE - Residues can be absent or present
  REGION 44..70: note = MISC_FEATURE - Residues can be absent or present
GGGSGGGSGGGSGGGSGGGSGGGSGGGSGGGSGGGSGGGSGGSGGSGGSGGSGGSGGSGGSGGSGGSGGS

SEQ ID NO: 163 - protein, 45 aa, synthetic construct
  REGION 1..45: note = GlySer linker
  REGION 5..40: note = MISC_FEATURE - Residues can be present or absent
GGGSGGGSGGGSGGGSGGGSGGGSGGGSGGGSGGGSGGGSGGGGS

SEQ ID NO: 164 - protein, 20 aa, synthetic construct
  REGION 1..20: note = GlySer linker
GGGGSGGGGSGGGGSGGGGS

SEQ ID NO: 165 - protein, 15 aa, synthetic construct
  REGION 1..15: note = GlySer linker
GGGGSGGGGSGGGGS

SEQ ID NO: 166 - protein, 10 aa, synthetic construct
  REGION 1..10: note = GlySer linker
GGGGSGGGGS

SEQ ID NO: 167 - protein, 5 aa, synthetic construct
  REGION 1..5: note = GlySer linker
GGGGS

SEQ ID NO: 168 - protein, 8 aa, synthetic construct
  REGION 1..8: note = GlySer linker
GGGSGGGS

SEQ ID NO: 169 - protein, 4 aa, synthetic construct
  REGION 1..4: note = GlySer linker
GGGS

SEQ ID NO: 170 - protein, 6 aa, synthetic construct
  REGION 1..6: note = GlySer linker
GGSGGS

SEQ ID NO: 171 - protein, 11 aa, synthetic construct
  REGION 1..11: note = GlySer linker
GGSGGGSGGSG

SEQ ID NO: 172 - protein, 10 aa, synthetic construct
  REGION 1..10: note = GlySer linker
GGSGGGSGSG

SEQ ID NO: 173 - protein, 8 aa, synthetic construct
  REGION 1..8: note = GlySer linker
GGSGGGSG

SEQ ID NO: 174 - protein, 5 aa, synthetic construct
  REGION 1..5: note = Spacer
  REGION 1..5: note = MISC_FEATURE - Entire sequence can be repeated n times wherein n is an integer including 1, 2, 3, 4, 5, 6, 7, 8, 9, or more
EAAAK

SEQ ID NO: 175 - protein, 6 aa, synthetic construct
  REGION 1..6: note = His tag
HHHHHH

SEQ ID NO: 176 - protein, 8 aa, synthetic construct
  REGION 1..8: note = Flag tag
DYKDDDDK

SEQ ID NO: 177 - protein, 8 aa, synthetic construct
  REGION 1..8: note = Xpress tag
DLYDDDDK

SEQ ID NO: 178 - protein, 15 aa, synthetic construct
  REGION 1..15: note = Avi tag
GLNDIFEAQKIEWHE

SEQ ID NO: 179 - protein, 26 aa, synthetic construct
  REGION 1..26: note = Calmodulin tag
KRRWKKNFIAVSAANRFKKISSSGAL

SEQ ID NO: 180 - protein, 9 aa, synthetic construct
  REGION 1..9: note = HA tag
YPYDVPDYA

SEQ ID NO: 181 - protein, 10 aa, synthetic construct
  REGION 1..10: note = Myc tag
EQKLISEEDL

SEQ ID NO: 182 - protein, 8 aa, synthetic construct
  REGION 1..8: note = STREP tag
WRHPQFGG

SEQ ID NO: 183 - protein, 8 aa, synthetic construct
  REGION 1..8: note = STREP tag II
WSHPQFEK

SEQ ID NO: 184 - protein, 13 aa, synthetic construct
  REGION 1..13: note = Softag 1
SLAELLNAGLGGS

SEQ ID NO: 185 - protein, 8 aa, synthetic construct
  REGION 1..8: note = Softag 3
TQDPSRVG

SEQ ID NO: 186 - protein, 14 aa, synthetic construct
  REGION 1..14: note = V5 tag
GKPIPNPLLGLDST

SEQ ID NO: 187 - skipped

SEQ ID NO: 188 - DNA, 63 bp, synthetic construct
  misc_feature 1..63: note = Signal peptide coding sequence
  source 1..63: mol_type = other DNA
ctgctgctcgtgaccagcctgctgctgtgcgaactgccccaccctgcctttctgctgatcccc

SEQ ID NO: 189 - DNA, 45 bp, synthetic construct
  misc_feature 1..45: note = GlySer linker coding sequence
  source 1..45: mol_type = other DNA
ggaggcggaggatctggcggagggggctctggaggaggaggatct

SEQ ID NO: 190 - protein, 260 aa, synthetic construct
  REGION 1..260: note = 1H10 scFv in VH-VL orientation
METDTLLLWVLLLWVPGSTGQVQLVQSGAEVKKPGASVKVSCKGSGYIFTSYDMHWVRQAPGQGLEWMGIIDPSGGSTSYAQKFQGRVTMTRDTSMSTVYMELSSLRSEDTAVYYCTRDYSWSYFDYWGQGTLVTVSSGGGGSGGGGSGGGGSAIQMTQSPSSLSASVGDRVTITCRASQGIRIYLGWYQQKPGKAPKLLIYATSSLQSGVPSRFSGSGSGTDFTLTISSLQPEDFATYYCLQDYNYPWTFGQGTKVEIK

SEQ ID NO: 191 - protein, 260 aa, synthetic construct
  REGION 1..260: note = 1H10 scFv in VL-VH orientation
METDTLLLWVLLLWVPGSTGAIQMTQSPSSLSASVGDRVTITCRASQGIRIYLGWYQQKPGKAPKLLIYATSSLQSGVPSRFSGSGSGTDFTLTISSLQPEDFATYYCLQDYNYPWTFGQGTKVEIKGGGGSGGGGSGGGGSQVQLVQSGAEVKKPGASVKVSCKGSGYIFTSYDMHWVRQAPGQGLEWMGIIDPSGGSTSYAQKFQGRVTMTRDTSMSTVYMELSSLRSEDTAVYYCTRDYSWSYFDYWGQGTLVTVSS

SEQ ID NO: 192 - protein, 259 aa, synthetic construct
  REGION 1..259: note = 1A9 scFv in VH-VL orientation
METDTLLLWVLLLWVPGSTGEVQLVESGGGLVQPGGSLRLSCAASGFTFSIYDMHWVRQATGKGLEWVSAIGTAGDTYYAGSVKGRFTISRENAKNSLYLQMNSLRAGDTAVYYCAREYSGYYFDYWGQGTLVTVSSGGGGSGGGGSGGGGSAIQMTQSPSSLSASVGDRVTITCRASQDIRNDLGWYQQKPGKAPKILIYGASSLQSGVPSRFSGSGSGTDFTFTISSLQPEDFATYYCLQEYNYPCTFGQGTKLEIK

SEQ ID NO: 193 - protein, 259 aa, synthetic construct
  REGION 1..259: note = 1A9 scFv in VL-VH orientation
METDTLLLWVLLLWVPGSTGAIQMTQSPSSLSASVGDRVTITCRASQDIRNDLGWYQQKPGKAPKILIYGASSLQSGVPSRFSGSGSGTDFTFTISSLQPEDFATYYCLQEYNYPCTFGQGTKLEIKGGGGSGGGGSGGGGSEVQLVESGGGLVQPGGSLRLSCAASGFTFSIYDMHWVRQATGKGLEWVSAIGTAGDTYYAGSVKGRFTISRENAKNSLYLQMNSLRAGDTAVYYCAREYSGYYFDYWGQGTLVTVSS

SEQ ID NO: 194 - protein, 260 aa, synthetic construct
  REGION 1..260: note = 1E6 scFv in VH-VL orientation
METDTLLLWVLLLWVPGSTGQVQLVESGGGVVQPGRSLRLSCAASGFTFSSYDIHWVRQAPGKGLEWVAVIWYDGSHNYYSDSVKGRFTISRDNSKNTLYLQMNSLRAEDTAVYYCARDYSGSYYDYWGQGTLVTVSSGGGGSGGGGSGGGGSAIQMTQSPSSLSASVGDRVTITCRASQGIRNDLGWYQQKPGKAPKLLIYAASNLQSGVPSRFSGSGSGTDFTLTISSLQPEDFATYYCLQDYSYPRTFGQGTKVEIK

SEQ ID NO: 195 - protein, 260 aa, synthetic construct
  REGION 1..260: note = 1E6 scFv in VL-VH orientation
METDTLLLWVLLLWVPGSTGAIQMTQSPSSLSASVGDRVTITCRASQGIRNDLGWYQQKPGKAPKLLIYAASNLQSGVPSRFSGSGSGTDFTLTISSLQPEDFATYYCLQDYSYPRTFGQGTKVEIKGGGGSGGGGSGGGGSQVQLVESGGGVVQPGRSLRLSCAASGFTFSSYDIHWVRQAPGKGLEWVAVIWYDGSHNYYSDSVKGRFTISRDNSKNTLYLQMNSLRAEDTAVYYCARDYSGSYYDYWGQGTLVTVSS

SEQ ID NO: 196 - protein, 262 aa, synthetic construct
  REGION 1..262: note = 2D3 scFv in VH-VL orientation
METDTLLLWVLLLWVPGSTGEVQLLESGGGLVQPGGSLSLSCAASGFTFSIYAMSWVRQAPGKGLEWVSAISDSGGTTYYADSVKGRFTISRDNSKNMLYLEMNSLRAEDTAIYYCAKRTRYFNGMDVWGQGTTVTVSSGGGGSGGGGSGGGGSEIVMTQSPATLSLSPGERATLSCRASQSGSSSFLSWYQQKPGQAPRLLIYGASTRATGIPARFSGSGSGTDFTLTISSLQPEDFAVYYCQQDYNLPFTFGPGTKVDIK

SEQ ID NO: 197 - protein, 262 aa, synthetic construct
  REGION 1..262: note = 2D3 scFv in VL-VH orientation
METDTLLLWVLLLWVPGSTGEIVMTQSPATLSLSPGERATLSCRASQSGSSSFLSWYQQKPGQAPRLLIYGASTRATGIPARFSGSGSGTDFTLTISSLQPEDFAVYYCQQDYNLPFTFGPGTKVDIKGGGGSGGGGSGGGGSEVQLLESGGGLVQPGGSLSLSCAASGFTFSIYAMSWVRQAPGKGLEWVSAISDSGGTTYYADSVKGRFTISRDNSKNMLYLEMNSLRAEDTAIYYCAKRTRYFNGMDVWGQGTTVTVSS

SEQ ID NO: 198 - DNA, 1092 bp, Homo sapiens
  source 1..1092: mol_type = other DNA
atgccgctgctgctactgctgcccctgctgtgggcaggggccctggctatggatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacagacttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctcaacgtcacctatgttccacagaacccaacaactggtatctttccaggagatggctcagggaaacaagagaccagagcaggagtggttcatggggccattggaggagctggtgttacagccctgctcgctctttgtctctgcctcatcttcttcatagtgaagacccacaggaggaaagcagccaggacagcagtgggcaggaatgacacccaccctaccacagggtcagcctccccgaaacaccagaagaagtccaagttacatggccccactgaaacctcaagctgttcaggtgccgcccctactgtggagatggatgaggagctgcattatgcttccctcaactttcatgggatgaatccttccaaggacacctccaccgaatactcagaggtcaggacccag

SEQ ID NO: 199 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 200 - DNA, 3288 bp, synthetic construct
  misc_feature 1..3288: note = 1H10-LvHv-intDS-41bb-3z-T-CD19t Top Strand
  source 1..3288: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagccaccatgctgctgctcgtgaccagcctgctgctgtgcgaactgccccaccctgcctttctgctgatccccgcgatacaaatgacgcaaagtcccagcagtttgtccgcctcagtaggcgaccgcgttacgattacgtgtagggcgtctcaagggatcaggatctatctgggctggtatcaacaaaagcctgggaaagccccaaagctccttatatatgcaacatcatccctgcaaagcggcgttccatcccgattcagtggttctggtagcggtacggacttcactctcacaatctcatctcttcaaccagaagactttgcgacgtattactgtttgcaagactacaattatccatggacgttcggccaaggcacgaaagtcgagataaagggaggcggaggatctggcggagggggctctggaggaggaggatctcaagtacagcttgttcaaagtggtgctgaagttaaaaagccaggggccagcgttaaggtatcctgcaagggaagtggttacatcttcacatcttacgacatgcactgggtacgacaggctcctggacagggtctggaatggatgggtatcatagacccctcaggaggatctacgagctatgcccaaaaatttcagggaagagtaacaatgaccagggacacgtccatgagcacagtctacatggaactcagcagtctcagatcagaggatacggcggtttactactgtactagggattattcatggagctatttcgactattggggacaaggaaccttggtaacagtgtcttcagagtctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatccaggccctaggatgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 201 - DNA, 3285 bp, synthetic construct
  misc_feature 1..3285: note = 1A9-LvHv-intDS-41bb-3z-T-CD19t Top Strand
  source 1..3285: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagccaccatgctgctgctcgtgaccagcctgctgctgtgcgaactgccccaccctgcctttctgctgatccccgcgattcagatgactcaatccccctcctctctctccgcgtccgtaggggatagggtgacaataacttgtagggcgagccaggacatccgcaatgacctcggctggtatcaacaaaaaccaggcaaggcacctaagatactgatttatggcgcgtcctccttgcaatccggggtgccgtctcggttcagtggttcaggtagtggtacggactttaccttcacaatctctagtctgcaaccggaggatttcgctacttactattgtctccaggagtataattacccctgtacatttgggcaaggcaccaagttggagataaaaggaggcggaggatctggcggagggggctctggaggaggaggatctgaagtgcagttggttgagtctggaggaggcctggtacagccgggtggtagtcttcggctttcctgtgctgctagcgggtttactttctccatatacgatatgcactgggtgaggcaagcgaccggaaaaggtctggagtgggtctcagcgatcggtacagctggcgatacttactatgcgggcagtgtcaagggacgattcaccataagccgcgaaaacgctaaaaattccctctacttgcaaatgaatagcctgcgagcgggggacaccgccgtatattattgtgctagagagtatagcggatattactttgactattggggtcaaggcactctggtaacggtgtctagcgagtctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatccaggccctaggatgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 202 - DNA, 3354 bp, synthetic construct
  misc_feature 1..3354: note = 1E6-LvHv-intDS-41bb-3z-T-CD19t Top Strand
  source 1..3354: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagccaccatgctgctgctcgtgaccagcctgctgctgtgcgaactgccccaccctgcctttctgctgatccccgcaatacaaatgacgcagtctcctagctcactttctgcaagcgtcggagaccgagttacaattacgtgtcgggcgagccagggaattcggaacgatctcggctggtatcaacagaaacccggcaaagcgccaaaattgcttatatacgcggcatcaaaccttcagagtggtgtgccgtcaagattcagtgggtcaggcagcggaactgactttaccctgactatctctagtctccaacccgaggacttcgcaacgtactattgcctgcaagattactcctacccgcgaacgttcggccaagggacaaaggttgagattaaaggaggcggaggatctggcggagggggctctggaggaggaggatctatgctgctgctcgtgaccagcctgctgctgtgcgaactgccccaccctgcctttctgctgatcccccaagtgcagttggttgaatccggtggcggtgtggttcaaccaggcaggagcttgagactttcatgtgcagcgtccggctttacattctccagctacgacatacattgggtccggcaggcgccaggaaagggcctcgaatgggtcgcggtaatatggtacgacggcagtcataactactacagtgattctgtaaaaggccgctttacgatttcacgcgacaacagcaagaatacactctatttgcaaatgaactctctgcgcgcggaagataccgccgtgtattattgtgcgcgggactacagcgggtcttactacgactactggggccaaggaacccttgtaacggtctctagcgagtctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatccaggccctaggatgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 203 - DNA, 3294 bp, synthetic construct
  misc_feature 1..3294: note = 2D3-LvHv-intDS-41bb-3z-T-CD19t Top Strand
  source 1..3294: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagccaccatgctgctgctcgtgaccagcctgctgctgtgcgaactgccccaccctgcctttctgctgatccccgagattgtaatgacgcagtctccagcgacgctttctcttagtccgggagaaagagccacactgtcctgccgggcgtcccaatccggttctagctcctttctgtcatggtatcaacagaagccaggtcaggcacctcgccttcttatttacggtgcatccactcgcgcgaccgggattcctgcaagattttccgggtctgggtctggcacagatttcacgttgactatcagtagtctgcagccagaggatttcgcagtctattactgtcaacaagactacaatcttcctttcacgtttggtcccggaactaaggttgatataaaaggaggcggaggatctggcggagggggctctggaggaggaggatctgaggtgcaattgctggaaagtggaggaggactcgtgcagcccggaggttcccttagcctttcttgcgctgcaagtgggtttacgttctctatatatgccatgtcttgggtgcggcaagcccccggaaaaggattggaatgggtatctgccattagtgattctgggggtacgacctattatgcagatagtgtaaaagggagattcactatctcacgcgacaattcaaagaatatgctttaccttgagatgaacagtcttcgagcagaggatacagccatatactattgcgctaaacgcacccgctacttcaacggaatggatgtatggggacagggtacaacagttactgtttctagcgagtctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatccaggccctaggatgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 204 - protein, 726 aa, synthetic construct
  REGION 1..726: note = CD33:CD22 4D protein
MPLLLLLPLLWAGALAMHHHHHHGGGDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 205 - DNA, 2178 bp, synthetic construct
  misc_feature 1..2178: note = CD33:CD22 4D nucleotides
  source 1..2178: mol_type = other DNA
atgcctctgctgctactgctacctctgctgtgggctggagccctggctatgcatcatcaccaccatcacggcggcggcgatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacagacttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctgaacgtcacctatgttccacagaacccaacaactggtatctttccaggagatggctcagggaaacaagagaccagagcaggagtggttcatccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccttggcacgctgggacttattcctgtgtggcagaaaacattcttggtactggacagaggggccctggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccaggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccggggccattggaggagctggtgttacagccctgctcgctctttgtctctgcctcatcttcttcatagtgaagacccacaggaggaaagcagccaggacagcagtgggcaggaatgacacccaccctaccacagggtcagcctccccgaaacaccagaagaagtccaagttacatggccccactgaaacctcaagctgttcaggtgccgcccctactgtggagatggatgaggagctgcattatgcttccctcaactttcatgggatgaatccttccaaggacacctccaccgaatactcagaggtcaggacccag

SEQ ID NO: 206 - protein, 552 aa, synthetic construct
  REGION 1..552: note = CD33:CD22 2D protein
MPLLLLLPLLWAGALAMHHHHHHGGGDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 207 - DNA, 1656 bp, synthetic construct
  misc_feature 1..1656: note = CD33:CD22 2D nucleotides
  source 1..1656: mol_type = other DNA
atgcctctgctgctactgctacctctgctgtgggctggagccctggctatgcatcatcaccaccatcacggcggcggcgatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacagacttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctgaacgtcacctatgttccacagaacccaacaactggtatctttccaggagatggctcagggaaacaagagaccagagcaggagtggttcatccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccaggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccggggccattggaggagctggtgttacagccctgctcgctctttgtctctgcctcatcttcttcatagtgaagacccacaggaggaaagcagccaggacagcagtgggcaggaatgacacccaccctaccacagggtcagcctccccgaaacaccagaagaagtccaagttacatggccccactgaaacctcaagctgttcaggtgccgcccctactgtggagatggatgaggagctgcattatgcttccctcaactttcatgggatgaatccttccaaggacacctccaccgaatactcagaggtcaggacccag

SEQ ID NO: 208 - protein, 280 aa, synthetic construct
  REGION 1..280: note = CD33 V-set construct (exon 3 and 4 deleted) protein
MPLLLLLPLLWAGALAMHHHHHHGGGDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 209 - DNA, 840 bp, synthetic construct
  misc_feature 1..840: note = CD33 V-set construct (exon 3 and 4 deleted) nucleotides
  source 1..840: mol_type = other DNA
atgcctctgctgctactgctacctctgctgtgggctggagccctggctatgcatcatcaccaccatcacggcggcggcgatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacatatgttccacagaacccaacaactggtatctttccaggagatggctcagggaaacaagagaccagagcaggagtggttcatggggccattggaggagctggtgttacagccctgctcgctctttgtctctgcctcatcttcttcatagtgaagacccacaggaggaaagcagccaggacagcagtgggcaggaatgacacccaccctaccacagggtcagcctccccgaaacaccagaagaagtccaagttacatggccccactgaaacctcaagctgttcaggtgccgcccctactgtggagatggatgaggagctgcattatgcttccctcaactttcatgggatgaatccttccaaggacacctccaccgaatactcagaggtcaggacccag

SEQ ID NO: 210 - protein, 17 aa, synthetic construct
  REGION 1..17: note = CD33 signal peptide
MPLLLLLPLLWAGALAM

SEQ ID NO: 211 - DNA, 51 bp, synthetic construct
  misc_feature 1..51: note = CD33 signal peptide coding sequence
  source 1..51: mol_type = other DNA
atgcctctgctgctactgctacctctgctgtgggctggagccctggctatg

SEQ ID NO: 212 - DNA, 18 bp, synthetic construct
  misc_feature 1..18: note = 6-histidine tag coding sequence
  source 1..18: mol_type = other DNA
catcatcaccaccatcac

SEQ ID NO: 213 - protein, 242 aa, synthetic construct
  REGION 1..242: note = CD33 extracellular domain
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVH

SEQ ID NO: 214 - DNA, 726 bp, synthetic construct
  misc_feature 1..726: note = CD33 extracellular domain coding sequence
  source 1..726: mol_type = other DNA
gatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacagacttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctgaacgtcacctatgttccacagaacccaacaactggtatctttccaggagatggctcagggaaacaagagaccagagcaggagtggttcat

SEQ ID NO: 215 - protein, 149 aa, synthetic construct
  REGION 1..149: note = CD33 extracellular domain lacking CD33 amino acids 140-232
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTYVPQNPTTGIFPGDGSGKQETRAGVVH

SEQ ID NO: 216 - DNA, 447 bp, synthetic construct
  misc_feature 1..447: note = CD33 extracellular domain lacking CD33 amino acids 140-232 coding sequence
  source 1..447: mol_type = other DNA
gatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacatatgttccacagaacccaacaactggtatctttccaggagatggctcagggaaacaagagaccagagcaggagtggttcat

SEQ ID NO: 217 - DNA, 13 bp, synthetic construct
  misc_feature 1..13: note = CD33 transmembrane domain
  source 1..13: mol_type = other DNA
gaggagvtaaccv

SEQ ID NO: 218 - DNA, 69 bp, synthetic construct
  misc_feature 1..69: note = CD33 transmembrane domain coding sequence
  source 1..69: mol_type = other DNA
ggggccattggaggagctggtgttacagccctgctcgctctttgtctctgcctcatcttcttcatagtg

SEQ ID NO: 219 - protein, 82 aa, synthetic construct
  REGION 1..82: note = CD33 intracellular domain
KTHRRKAARTAVGRNDTHPTTGSASPKHQKKSKLHGPTETSSCSGAAPTVEMDEELHYASLNFHGMNPSKDTSTEYSEVRTQ

SEQ ID NO: 220 - DNA, 246 bp, synthetic construct
  misc_feature 1..246: note = CD33 intracellular domain coding sequence
  source 1..246: mol_type = other DNA
aagacccacaggaggaaagcagccaggacagcagtgggcaggaatgacacccaccctaccacagggtcagcctccccgaaacaccagaagaagtccaagttacatggccccactgaaacctcaagctgttcaggtgccgcccctactgtggagatggatgaggagctgcattatgcttccctcaactttcatgggatgaatccttccaaggacacctccaccgaatactcagaggtcaggacccag

SEQ ID NO: 221 - protein, 353 aa, synthetic construct
  REGION 1..353: note = Portion of CD22 extracellular domain that contains CD22 domains defined as Ig-like C2-type 3, Ig-like C2-type 4, Ig-like C2-type 5, Ig-like C2-type 6
PEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPET

SEQ ID NO: 222 - DNA, 1059 bp, synthetic construct
  misc_feature 1..1059: note = Portion of CD22 extracellular domain that contains CD22 domains defined as Ig-like C2-type 3, Ig-like C2-type 4, Ig-like C2-type 5, Ig-like C2-type 6 coding sequence
  source 1..1059: mol_type = other DNA
ccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccttggcacgctgggacttattcctgtgtggcagaaaacattcttggtactggacagaggggccctggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccaggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagacc

SEQ ID NO: 223 - protein, 179 aa, synthetic construct
  REGION 1..179: note = Portion of CD22 extracellular domain that contains CD22 domains defined as Ig-like C2-type 5, Ig-like C2-type 6
PRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPET

SEQ ID NO: 224 - DNA, 537 bp, synthetic construct
  misc_feature 1..537: note = Portion of CD22 extracellular domain that contains CD22 domains defined as Ig-like C2-type 5, Ig-like C2-type 6 coding sequence
  source 1..537: mol_type = other DNA
ccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccaggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagacc

SEQ ID NO: 225 - protein, 22 aa, synthetic construct
  REGION 1..22: note = 1H10, 1A9, 1E6, and/or 1B9 light chain signal peptide
MDMRVPAQLLGLLLLWLPGARC

SEQ ID NO: 226 - protein, 20 aa, synthetic construct
  REGION 1..20: note = 1D2 light chain signal peptide
MRVPAQLLGLLLLWLPGARC

SEQ ID NO: 227 - protein, 22 aa, synthetic construct
  REGION 1..22: note = 1H8 light chain signal peptide
MDMRLPAQLLGLLMLWVPASRG

SEQ ID NO: 228 - protein, 22 aa, synthetic construct
  REGION 1..22: note = 2D3 light chain signal peptide
MEPWKPQHSFFFLLLWLPDSTG

SEQ ID NO: 229 - protein, 19 aa, synthetic construct
  REGION 1..19: note = 1H10 heavy chain signal peptide
MDWTWRVFCLLAVAPGVHS

SEQ ID NO: 230 - protein, 19 aa, synthetic construct
  REGION 1..19: note = 1A9 heavy chain signal peptide
MELGLSWVFLVAILEGVQC

SEQ ID NO: 231 - protein, 19 aa, synthetic construct
  REGION 1..19: note = 1E6 and/or 2E3 heavy chain signal peptide
MEFGLSWVFLVALLRGVQC

SEQ ID NO: 232 - protein, 19 aa, synthetic construct
  REGION 1..19: note = 1D2 heavy chain signal peptide
MESGLSWVFLVALLRGVQC

SEQ ID NO: 233 - protein, 19 aa, synthetic construct
  REGION 1..19: note = 1B9 heavy chain signal peptide
MEFGLSWVFLIALLRGVQC

SEQ ID NO: 234 - protein, 22 aa, synthetic construct
  REGION 1..22: note = 1H8 heavy chain signal peptide
MLLLVTSLLLCELPHPAFLLIP

SEQ ID NO: 235 - protein, 19 aa, synthetic construct
  REGION 1..19: note = 2D3 heavy chain signal peptide
MEFGLSWLFLVAILKGVQC

SEQ ID NO: 236 - DNA, 738 bp, synthetic construct
  misc_feature 1..738: note = My96 Coding Sequence
  source 1..738: mol_type = other DNA
gagatcgtgctgacacagagccctggaagcctggccgtgtctcctggcgagcgcgtgacaatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagagaggcggcggaggctctggcggaggcggatctagtggcggaggatctcaggtgcagctgcagcagcctggcgccgaggtcgtgaaacctggcgcctctgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtcatct

SEQ ID NO: 237 - DNA, 9278 bp, synthetic construct
  misc_feature 1..9278: note = My96_int_41BB_3z_TCD19 Coding Sequence
  source 1..9278: mol_type = other DNA
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagccaccatgctgctgctcgtgaccagcctgctgctgtgcgaactgccccaccctgcctttctgctgatccccgagatcgtgctgacacagagccctggaagcctggccgtgtctcctggcgagcgcgtgacaatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagagaggcggcggaggctctggcggaggcggatctagtggcggaggatctcaggtgcagctgcagcagcctggcgccgaggtcgtgaaacctggcgcctctgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtcatctgagtctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatccaggccctaggatgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 238 - protein, 107 aa, synthetic construct
  REGION 1..107: note = 1H10 variable light chain
AIQMTQSPSSLSASVGDRVTITCRASQGIRIYLGWYQQKPGKAPKLLIYATSSLQSGVPSRFSGSGSGTDFTLTISSLQPEDFATYYCLQDYNYPWTFGQGTKVEIK

SEQ ID NO: 239 - protein, 118 aa, synthetic construct
  REGION 1..118: note = 1H10 variable heavy chain
QVQLVQSGAEVKKPGASVKVSCKGSGYIFTSYDMHWVRQAPGQGLEWMGIIDPSGGSTSYAQKFQGRVTMTRDTSMSTVYMELSSLRSEDTAVYYCTRDYSWSYFDYWGQGTLVTVSS

SEQ ID NO: 240 - protein, 107 aa, synthetic construct
  REGION 1..107: note = 1A9 variable light chain
AIQMTQSPSSLSASVGDRVTITCRASQDIRNDLGWYQQKPGKAPKILIYGASSLQSGVPSRFSGSGSGTDFTFTISSLQPEDFATYYCLQEYNYPCTFGQGTKLEIK

SEQ ID NO: 241 - protein, 117 aa, synthetic construct
  REGION 1..117: note = 1A9 variable heavy chain
EVQLVESGGGLVQPGGSLRLSCAASGFTFSIYDMHWVRQATGKGLEWVSAIGTAGDTYYAGSVKGRFTISRENAKNSLYLQMNSLRAGDTAVYYCAREYSGYYFDYWGQGTLVTVSS

SEQ ID NO: 242 - protein, 107 aa, synthetic construct
  REGION 1..107: note = 1E6 variable light chain
AIQMTQSPSSLSASVGDRVTITCRASQGIRNDLGWYQQKPGKAPKLLIYAASNLQSGVPSRFSGSGSGTDFTLTISSLQPEDFATYYCLQDYSYPRTFGQGTKVEIK

SEQ ID NO: 243 - protein, 118 aa, synthetic construct
  REGION 1..118: note = 1E6 variable heavy chain
QVQLVESGGGVVQPGRSLRLSCAASGFTFSSYDIHWVRQAPGKGLEWVAVIWYDGSHNYYSDSVKGRFTISRDNSKNTLYLQMNSLRAEDTAVYYCARDYSGSYYDYWGQGTLVTVSS

SEQ ID NO: 244 - protein, 107 aa, synthetic construct
  REGION 1..107: note = 1D2 variable light chain
AIQMTQSPSSLSASVGDRVTITCRASQGIRNDLGWYQQKPGKAPELLIYATSSLQSGVPSRFSGSGSGTDFTLIISSLQPEDFATYYCLQDYSYPRTFGQGTKVEIK

SEQ ID NO: 245 - protein, 118 aa, synthetic construct
  REGION 1..118: note = 1D2 variable heavy chain
QVQLVESGGGVVQPGRSLRLSCAASGFTFSSYDIHWVRQAPGKGLEWVAVIWYDGSQKYYADSVKGRFTISRDNSKNTLYLQMNSLRAEDTAVYYCARDYSGSYYDYWGQGTLVTVSS

SEQ ID NO: 246 - protein, 107 aa, synthetic construct
  REGION 1..107: note = 1B9 variable light chain
AIQMTQSPSSLSASVGDRVTITCRASQDIRNDLGWYLQRPGKAPKLLIYAASSLQSGVPSRFSGSGSGTDFTLTISSLQPEDFATYYCLQDYSYPRTFGQGTTVEIK

SEQ ID NO: 247 - protein, 118 aa, synthetic construct
  REGION 1..118: note = 1B9 variable heavy chain
QVQLVESGGGVVQPGRSLRLSCAASGFIFSSYDIHWVRQAPGKGLEWVAVIWYDGSHNYYSDSVKGRFTISRDNSKNTLYLQMNSLRAEDTAVYYCARDYSGSYFDYWGQGTLVTVSS

SEQ ID NO: 248 - protein, 107 aa, synthetic construct
  REGION 1..107: note = 1H8 variable light chain
EIVLTQSPDFQSVTPKEKVTITCRASQNIGGNLHWYQQKPDQSPKLLIRYATQPFSGVPSRFGGSGSGTDFTLTINSLEAEDAATYYCHQSSSLPLTFGGGTKVEIK

SEQ ID NO: 249 - protein, 120 aa, synthetic construct
  REGION 1..120: note = 1H8 variable heavy chain
QVQLVESGGGVVQPGGSLRLSCAASGFTFGSYGMHWVRQAPGKGLEWVAVIWYDGSNEYYADSVKGRFTVSRDNSKHTLYLQMNRLRAEDTAVYYCARDLDYDSSGGDYWGQGILVLVSS

SEQ ID NO: 250 - protein, 108 aa, synthetic construct
  REGION 1..108: note = 2D3 variable light chain
EIVMTQSPATLSLSPGERATLSCRASQSGSSSFLSWYQQKPGQAPRLLIYGASTRATGIPARFSGSGSGTDFTLTISSLQPEDFAVYYCQQDYNLPFTFGPGTKVDIK

SEQ ID NO: 251 - protein, 119 aa, synthetic construct
  REGION 1..119: note = 2D3 variable heavy chain
EVQLLESGGGLVQPGGSLSLSCAASGFTFSIYAMSWVRQAPGKGLEWVSAISDSGGTTYYADSVKGRFTISRDNSKNMLYLEMNSLRAEDTAIYYCAKRTRYFNGMDVWGQGTTVTVSS

SEQ ID NO: 252 - protein, 107 aa, synthetic construct
  REGION 1..107: note = 2E3 variable light chain
EIVLTQSPGTLSLSPGERATLSCRASQSVSSSYLAWYQQKPGQAPRLLIYGTSSRATGIPDRFSGSGSGTDFTLTISRLEPEDFAVYYCQQYGSSPTFGGGTKVEIK

SEQ ID NO: 253 - protein, 122 aa, synthetic construct
  REGION 1..122: note = 2E3 variable heavy chain
QVCLVESGGGVVQPGKSLRLSCAASGFTFSSYGMHWVRQAPGKGLEWVAVIWYGGSNKYYADSVKGRFTISRDNSKNTLYLQMNSLRAEDTAVYYCARDGTGENYYYYVMDVWGQGTTVTVS

SEQ ID NO: 254 - protein, 531 aa, synthetic construct
  REGION 1..531: note = V-set directed CD33/CD3 BsAb
METDTLLLWVLLLWVPGSTGQVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 255 - protein, 505 aa, synthetic construct
  REGION 1..505: note = V-set directed CD33/CD3 BsAb without leader sequence or His tag
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 256 - protein, 250 aa, synthetic construct
  REGION 1..250: note = V-set directed CD33 scFv
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 257 - DNA, 750 bp, synthetic construct
  misc_feature 1..750: note = V-set directed CD33 scFv coding sequence
  source 1..750: mol_type = other DNA
caagttcagctcgtgcagagtggtgcagaggtcaagaagcctggagagagcgtcaaggtcagctgtaaagcatctggctatacattcactaattacggaatgaactgggtcaagcaggcgccaggtcagggacttgaatggatgggctggataaacacatatacaggagagccaacttatgctgacaaattccagggtagagtcacgatgacgacggacacatcaacctccaccgcgtatatggaaatcaggaatttgggcggagacgatacagcggtttactactgcgcccgatggagttggtctgatggctattatgtgtatttcgactactggggtcagggtacaagcgtcacagtaagttcaggaggcggaggatctggcggagggggctctggaggaggaggatctgatattgtaatgacccaatcccctgactcattgacagtatccctcggagagcggaccactataaactgcaaatccagccagtctgtattggactccagcaccaacaaaaatagccttgcgtggtatcagcaaaagccgggtcaaccacccaagctgctcttgagttgggcgagtaccagagagagtgggatacccgacaggtttagtggatctggctctggcaccgattttacgcttacaatcgacagtccgcaacccgaagactccgcgacgtactactgtcagcaatctgcacactttccaataaccttcgggcaagggacacggctggagatcaaa

SEQ ID NO: 258 - protein, 6 aa, synthetic construct
  REGION 1..6: note = CDRH2 of 2D3
SDSGGT

(19) United States
(12) Patent Application Publication
(10) Pub. No.: US 2026/0167738 A1
(43) Pub. Date: Jun. 18, 2026

(54) CHIMERIC ANTIGEN RECEPTORS TARGETING CD33

(71) Applicant: Fred Hutchinson Cancer Center, Seattle, WA (US)

(72) Inventors: Cameron J. Turtle, NSW (AU); Roland B. Walter, Seattle, WA (US); George S. Laszlo, Seattle, WA (US); Salvatore Fiorenza, Seattle, WA (US)

(73) Assignee: Fred Hutchinson Cancer Center, Seattle, WA (US)

(21) Appl. No.: 19/531,427

(22) Filed: Feb. 5, 2026

Related U.S. Application Data
(63) Continuation of application No. 17/907,648, now Pat. No. 12,570,758.
(60) Provisional application No. 63/003,196, filed on Mar. 31, 2020.

(51) Int. Cl. C07K 16/28; A61K 40/11; A61K 40/31; A61K 40/42; C07K 14/705; C07K 14/725; C12N 15/63

(52) U.S. Cl. CPC C07K 16/2896; A61K 40/11; A61K 40/31; A61K 40/421; C07K 14/7051; C07K 14/70521; C12N 15/63; A61K 2239/25; A61K 2239/48; C07K 2317/526; C07K 2317/565; C07K 2317/622; C07K 2319/03

(57) ABSTRACT

Chimeric antigen receptors (CARs) with binding domains derived from a novel suite of human CD33-binding antibodies are described. The CARs include optimized short and intermediate spacer regions. The current disclosure also provides methods of cell expansion/activation processes utilizing IL-2, IL-7, IL-15, and/or IL-21 that improve cellular proliferation and cell lysis of the CARs as described.

### CROSS REFERENCE TO RELATED APPLICATION

This application is a continuation application which claims priority to U.S. National Phase patent application Ser. No. 17/907,648, filed on Sep. 28, 2022, which claims priority to International Patent Application No. PCT/US2021/025255, filed on Mar. 31, 2021, which claims priority to U.S. Provisional Patent Application No. 63/003,196 filed on Mar. 31, 2020, the entire contents each of which are incorporated by reference herein.

### STATEMENT REGARDING FEDERALLY SPONSORED RESEARCH OR DEVELOPMENT

This invention was made with government support under CA234203 and CA245594 awarded by the National Institutes of Health. The government has certain rights in the invention.

### REFERENCE TO SEQUENCE LISTING

The Sequence Listing associated with this application is provided in text format in lieu of a paper copy, and is hereby incorporated by reference into the specification. The name of the text file containing the Sequence Listing is 3K11479.XML. The xml file is 401,408 bytes, was created on Feb. 3, 2026, and is being submitted electronically via Patent Center.

### FIELD OF THE DISCLOSURE

The current disclosure provides chimeric antigen receptors (CARs) with binding domains derived from a novel suite of human CD33-binding antibodies that can include optimized short and intermediate spacer regions. The current disclosure also provides methods of cell expansion/activation processes utilizing IL-2, IL-7, IL-15, and/or IL-21 that improve cellular proliferation and cell lysis of the CARs as described.

### BACKGROUND OF THE DISCLOSURE

According to the World Health Organization, cancer is the second leading cause of death globally, and was responsible for an estimated 9.6 million deaths in 2018. Acute myeloid leukemia (AML) is a type of cancer resulting from a malignancy of clonal, proliferative myeloid blast cells. There are 20,000 new cases of AML per year in the United States and 11,000 deaths from AML each year (Siegel, et al., 2021, CA Cancer J Clin. 71(1): 7-33). Although high complete remission rates can be achieved in younger patients with AML with conventional chemotherapy at rates of 60% to 80% (Döhner, et al., 2017. Blood. 129(4): 424-447), treatment outcomes for older patients, at the age of 65 or older, remains unsatisfactory with as many as 70% of patients dying of their disease within a year of diagnosis (Meyers, et al., Appl Health Econ Health Policy, 11:275-286, 2013). Unfortunately, because of the chemo-refractoriness of leukemic stem cells, relapse after conventional therapy is common (Eppert, et al., 2011. Nat. Med. 17(9): 1086-1093) and current treatment options for relapsed/refractory (R/R) AML are dismal, resulting in less than 30% overall survival at 12 months.

For many years, the chosen treatments for cancer have been surgery, chemotherapy, and/or radiation therapy. In recent years, more targeted therapies have emerged to specifically target cancer cells by identifying and exploiting specific molecular and/or immunophenotypic changes seen primarily in those cells. For example, many cancer cells preferentially express particular markers on their cellular surfaces and these markers have provided targets for antibody-based therapeutics.

CD33 is a member of the sialic acid binding, immunoglobulin-like lectin (SIGLEC) protein family. It is a 67-kDa glycosylated transmembrane protein. CD33 (also known as SIGLEC-3) is a myeloid differentiation antigen that is found at least on some leukemic cells in almost all patients with AML and, perhaps, on AML stem cells in some cases. Based on this broad expression pattern, CD33 has been widely pursued as a therapeutic target in AML. Recent data from several randomized studies have demonstrated that the CD33 antibody-drug conjugate, gemtuzumab ozogamicin (GO), improves survival when added to chemotherapy in defined subsets of patients with newly diagnosed AML. This data has validated CD33 as the first (and so far, only) target for immunotherapy in AML. In parallel to the development of new, more effective CD33-directed therapeutics (e.g., antibody-drug conjugates, radioimmunoconjugates, bispecific antibodies, chimeric antigen receptor [CAR]-modified T cells) to overcome the limitations noted with GO, interest has grown in CD33 as a drug target for other malignant and non-malignant disorders. These efforts include the targeting of CD33 splice variants not recognized by GO as well as the targeting of CD33+ tumor cells in other hematologic malignancies, CD33+ myeloid-derived suppressor cells (MDSCs) in a variety of diseases, and normal CD33+ microglial cells in Alzheimer disease (Walter, Expert Opin Biol Ther. 2020, 20(9):955-958).

The full length CD33 protein (CD33^{FL}) is characterized by an amino-terminal, membrane-distant V-set immunoglobulin (Ig)-like domain and a membrane-proximal C2-set Ig-like domain in its extracellular portion (FIG. 2). Shorter isoforms of CD33 exist. A shorter isoform of CD33 includes one variant that lacks exon 2, which encodes the V-set domain (CD33^{ΔE2}). At least at the mRNA level, CD33^{ΔE2} is broadly expressed in myeloid cells in the bone marrow and peripheral blood of patients with AML. Currently, however, almost all commercially and clinically available CD33 antibodies recognize the immune-dominant V-set Ig-like domain. This means that these antibodies would not recognize shorter forms of CD33 that lack the V-set domain such as CD33^{ΔE2}. This may explain the observation made in one clinical trial in pediatric AML that patients with a single nucleotide polymorphism in the CD33 gene that leads to preferential transcription of CD33^{ΔE2} and reduced translation of CD33^{FL} did not benefit from the addition of GO (which also binds to the V-set domain of CD33) to intensive chemotherapy.

Beyond antibody-based therapeutics, significant progress has been made in genetically engineering T cells of the immune system to target and kill unwanted cell types, such as cancer cells. Many of these T cells have been genetically engineered to express a chimeric antigen receptor (CAR). CARs are proteins including several distinct subcomponents that allow the genetically modified T cells to recognize and kill cancer cells. The subcomponents include at least an extracellular component and an intracellular component. The extracellular component includes a binding domain that specifically binds a marker that is preferentially present on the surface of unwanted cells (e.g., CD33). When the binding domain binds such markers, the intracellular component signals the T cell to destroy the bound cell. CARs additionally include a transmembrane domain that can link the extracellular component to the intracellular component.

Other subcomponents that can increase a CAR's function can also be used. For example, spacer regions can provide a CAR with additional conformational flexibility, often increasing the binding domain's ability to bind the targeted cell marker. The appropriate length of a spacer region within a particular CAR can depend on numerous factors including how close or far a targeted marker is located from the surface of an unwanted cell's membrane.

When performed ex vivo, genetically modifying T cells can involve numerous cell manipulation steps, and it has been observed that different manipulation conditions can affect the cancer-cell killing properties of the cells. Thus, in designing CARs and genetically modifying cells to express them, numerous considerations must be taken into account, including: targeted cell marker; presence and/or length of spacer; and ex vivo manipulation procedures.

### SUMMARY OF THE DISCLOSURE

The current disclosure provides chimeric antigen receptors (CARs) for the treatment of CD33-related disorders. The CARs include binding domains derived from a novel suite of anti-CD33 antibodies. In particular embodiments, the CARs include a binding domain that binds CD33 regardless of which CD33 variant a patient expresses (CD33^{FL} or CD33^{ΔE2}). These CD33 binding domains are referred to as “pan” binders. In particular embodiments, the pan binders bind the membrane-proximal C2-set Ig-like domain of CD33 (see FIG. 2). In particular embodiments, these pan binders are derived from antibodies: 1H10, 1A9, 1E6, 1D2, and 1B9 and can include single chain variable fragments of these antibody binding domains. Additional newly developed CD33 targeting antibodies disclosed herein bind the V-set domain of CD33. These antibodies include 1H8, 2D3, and 2E3 and provide additional CAR-based therapeutic options for patients that express CD33^{FL}.

In particular embodiments, the current disclosure provides CARs with a short or intermediate spacer region. In particular embodiments, the short spacer region includes the hinge region of IgG4 (12 amino acids). In particular embodiments, the intermediate spacer region includes the hinge region and the CH3 domain of IgG4 (collectively, 131 amino acids).

In particular embodiments, the current disclosure provides expanding and activating T cells genetically modified to express a CAR disclosed herein utilizing a combination of the cytokines IL-7, IL-15, and IL-21. In particular embodiments, the current disclosure provides expanding and activating T cells genetically modified to express a CAR disclosed herein utilizing a combination of cytokines including IL-2.

The CARs disclosed herein can be used in the treatment of acute myeloid leukemia (AML) and other CD33+ disorders.

### BRIEF DESCRIPTION OF THE FIGURES

FIG. 1. Expression of CD33 acute myeloid leukemia (AML) and normal hematopoietic cells indicating that CD33 is over-expressed in AML in comparison to hematopoietic stem cells. Relative expression data collated from bloodspot.eu. HSPC, hematopoietic stem/progenitor cell; MPP, multipotent progenitor. ****p<0.0001, ns not significant by multiple T-test using the two-stage linear step-up procedure of Benjamini, Krieger and Yekutieli, with Q=1%.

FIG. 2. Diagram of full-length CD33 (CD33^{FL}) and CD33 with deletion of exon 2, resulting deletion of V-set domain (CD33^{ΔE2}). Depicted are an antibody that binds CD33^{FL} only (anti-CD33^{FL}), CD33^{ΔE2} only (anti_CD33^{ΔE2}), or CD33^{FL} and CD33^{ΔE2} (anti-CD33^{FL}+ or anti-CD33^{PAN}).

FIG. 3. Schematic of CD33^{FL} and artificial CD33 molecules with deletion of exons 3 and 4, resulting in membrane proximal relocation of the V-set domain (CD33^{ΔE34}) or insertion of either 2 C2-set domains of CD22 (“CD33^{FL}+CD22 2D”) or 4 C2-set domains of CD22 (“CD33^{FL}+CD22 4D”). CD33^{ΔE34} was engineered using site-directed mutagenesis to splice out CD33 amino acids (aa) 140-232 of the human CD33^{FL} extracellular domain (ECD). CD33^{FL}+CD22 4D was generated using the endogenous CD33 signal peptide (aa 1-17), a 6-histidine tag, 3× glycine linker, the human CD33 ECD (aa 18-259), a portion of the human CD22 ECD including C2-type domains 3-6 (aa 331-683), the CD33 transmembrane domain, and the CD33 intracellular domain (aa 260-364). CD22 aa 331-504 (C2-type domains 3 and 4) were removed from CD33^{FL}+CD22 24 to generate CD33^{FL}+CD22 2D.

FIG. 4. Cloning strategy for generation of CD33 CAR expression constructs. (i) Supernatants from antibody-secreting mouse hybridoma cells were screened for pan-CD33 binding activity, then screened for lack of binding to CD33 null cell lines, and best individual hybridomas selected, antibody isotyping performed, RNA extracted and used for rapid amplification of cDNA ends (RACE) amplification (Takara) and subsequent isotype-specific polymerase chain reaction (PCR)-based amplification. (ii) Cloning of antibody variable region sequences was performed utilizing either pRACE (Takara) or TOPO (Invitrogen) standard cloning vectors. (iii) Plasmid DNA was purified from individual bacterial colonies, and Sanger DNA sequencing was performed to obtain at least 3 individual identical cDNA sequences corresponding to each antibody variable region for both the antibody heavy and light chains. (iv) Antibody variable region cDNA sequences were translated into amino acid sequences using the ExPASy.org translate tool. (v) Amino acid sequences for each individual antibody variable region were submitted to Integrated DNA Technologies (IDT, Coralville, IA) website for human codon optimization. (vi) Codon optimized single chain variable regions (scFvs) nucleic acid sequences were then combined with nucleic acid sequences contained within the CAR backbone lentiviral backbone-41BB-3z-T-CD19t, specifically, sequences within the EF1 promoter that flank the 5′ end of the granulocyte-macrophage colony-stimulating factor receptor (GM-CSFR) signal peptide, including the GM-CSFR signal peptide were added 5′ to the antibody HC variable region, followed by a flexible Gly-Ser linker sequence, and then the antibody LC variable region, and then linker sequence from the CAR backbone lentiviral backbone-41BB-3z-T-CD19t. (vii) Complete nucleotide sequences were then submitted to IDT for synthesis of gBlocks. (viii) gBlocks were cloned in TOPO, and nucleic acid sequences confirmed by Sanger DNA sequencing. (ix) All gBlocks were generated to include universal priming sequences, CAR_universal_forward primer and CAR_universal_reverse primer. (x) Next, PCR amplification was performed using an appropriate TOPO vector as template, proofreading DNA polymerase, and using CAR_universal_forward primer and a reverse primer selected from 2 options, CAR_universal_rev_sh or CAR_universal_rev_int. In these primers, rev indicates reverse, sh indicates the short linker, and int indicates the intermediate length linker within the CAR construct. (xi) Recipient plasmid (lentiviral backbone-41BB-3z-T-CD19t with sh, or int linker) was digested with RsrII/NheI and cut plasmid and PCR products were gel purified. (xii) Gibson assembly (NEB) was performed using RsrII/NheI digested recipient plasmid (lentiviral backbone-41BB-3z-T-CD19t with sh, or int linker) and appropriate PCR amplicon. (xiii) Sanger DNA sequencing verification was used to confirm the scFv and linker contained in each plasmid. (vii) Maxi prep and lentiviral packaging followed.

FIGS. 5A-5C. Reducing the binding distance from cell membrane enhances the anti-tumor efficacy of CD33/CD3 BsAbs against human myeloid leukemia cells. Human CD33+ myeloid leukemia cell lines ((5A) ML-1, (5B) HL-60, (5C) K562) with CRISPR/Cas9-mediated deletion of the endogenous CD33 locus were engineered to overexpress either CD33^{FL} or CD33^{ΔE3}-4 via lentiviral gene transfer. Relative expression of the target proteins was flow cytometrically assessed via V-set domain CD33 antibody, P67.6, with representative histograms shown in the bottom right panel. Cells were then treated with a V-set domain-targeting CD33/CD3 BsAb at a concentration of 1000 pg/mL and healthy donor T cells enriched from unstimulated peripheral blood mononuclear cells collected from healthy adult volunteers at the effector:target (E:T) cell ratios shown (top panel). Myeloid cells were also treated with gemtuzumab ozogamicin (GO) at the concentrations shown (bottom left panel). Cytotoxicity was quantified flow cytometrically after 2 days (for BsAbs) or 3 days (for GO) as a change in the percentage of dead cells as measured by 4′,6-diamidino-2-phenylindole (DAPI) staining. The anti-V-set domain-directed CD33/CD3 BsAb was constructed in the scFv-scFv format using a construct referred to herein as RC1 or A3 that utilizes the sequence as set forth in SEQ ID NO: 254 and described in United States patent application publication US 2016/0317657 A1. *p<0.05; **p<0.01; ***p<0.001.

FIG. 6. Reducing the binding distance from cell membrane enhances the anti-tumor efficacy of CD33/CD3 BsAbs against human acute lymphoblastic leukemia cells engineered to express CD33 proteins. The human CD33^{neg} acute lymphoblastic leukemia (ALL) cell line RS4;11 was engineered to overexpress either CD33^{FL} or CD33^{ΔE34} via lentiviral gene transfer. Relative expression of the target proteins was flow cytometrically assessed via V-set domain CD33 antibody, P67.6, with representative histograms shown in the bottom panel. Cells were then treated with a V-set domain-targeting CD33/CD3 BsAb at a concentration of 1000 pg/mL and healthy donor T cells enriched from unstimulated peripheral blood mononuclear cells collected from healthy adult volunteers at the effector:target (E:T) cell ratios shown (top panel). Cytotoxicity was quantified flow cytometrically after 2 days as a change in the percentage of dead cells as measured by 4′,6-diamidino-2-phenylindole (DAPI) staining. The anti-V-set domain-directed CD33/CD3 BsAb was constructed in the scFv-scFv format using a construct referred to herein as RC1 or A3 that utilizes the sequence as set forth in SEQ ID NO: 254 and described in United States patent application publication US 2016/0317657 A1. *p<0.05; **p<0.01; ***p<0.001.

FIG. 7. Increasing the binding distance from cell membrane reduces the anti-tumor efficacy of CD33/CD3 BsAbs. Human myeloid leukemia cell lines (ML-1 [upper panel], K562 [lower panel]) with CRISPR/Cas9-mediated deletion of the endogenous CD33 locus were engineered to overexpress CD33^{FL} CD33^{FL}+CD22 2D or CD33^{FL}+CD22 4D via lentiviral gene transfer. Relative expression of the CD33 constructs was flow cytometrically assessed using the V-set domain CD33 antibody, P67.6 (right panel). Cells were then treated with a V-set domain-targeting CD33/CD3 BsAb at the concentrations shown (in pg/mL) and healthy donor T cells enriched from unstimulated peripheral healthy donor blood mononuclear cells at an E:T cell ratio of 1:1. Cytotoxicity was quantified flow cytometrically after 2 days as a change in the percentage of dead cells as measured by 4′,6-diamidino-2-phenylindole (DAPI) staining. The anti-V-set domain-directed CD33/CD3 BsAb was constructed in the scFv-scFv format using a construct referred to herein as RC1 or A3 that utilizes the sequence as set forth in SEQ ID NO: 254 and described in United States patent application publication US 2016/0317657 A1. *p<0.05; **p<0.01; ***p<0.001.

FIG. 8. Reducing the binding distance from cell membrane enhances the anti-tumor efficacy of CD33 chimeric antigen receptor (CAR) T cells. The human myeloid leukemia cell line K562 with CRISPR/Cas9-mediated deletion of the endogenous CD33 locus was engineered to overexpress CD33^{FL} or CD33^{ΔE3-4} via lentiviral gene transfer. Relative expression of the target proteins was flow cytometrically assessed via V-set domain CD33 antibody, P67.6, with representative histograms. The efficacy of V-set domain-directed CAR T cells was assessed in a chromium⁵¹ release. For CAR T cell generation, healthy donor negative selected human CD8+ T cells were transduced with an epHIV7 lentivirus encoding the scFv from the CD33^{V-set}/CD3 BsAb described in FIGS. 3, 5A-5C, and 6 linked to an IgG₄ CH3 domain spacer, CD28-transmembrane domain, CD3zeta and 4-1BB intracellular signaling domain and truncated CD19 (tCD19) transduction marker. tCD19 CD8+ CAR-T cells were sorted and expanded in IL-7 and IL-15 (10 ng/mL; Peprotech, Rocky Hill, NJ, USA) each for 14 days with media and cytokine changes every other day. CAR-T cell cytotoxicity was assessed following incubation with chromium⁵¹ labelled targets for 4 hours.

FIG. 9. AML cell lines show different surface expression of CD33. CD33 expression as measured by Quantibrite-PE was measured by staining human AML cell lines with PE-conjugated p67.6 antibody.

FIGS. 10A-10C. Gene maps of chimeric antigen receptor (CAR) inserted into lentiviral vector backbones. VL and VH domain represent variable light (VL) and variable heavy (VH) domain of single chain variable fragments (scFv). Short (10A), intermediate (10B) and long (10C) domains differ by insertion of hinge only (short); hinge and CH3 domain (intermediate); and hinge, CH3 and CH2 domains (long) of human IgG4. All IgG4 domains contain mutated sequences to prevent binding to the human Fc receptor. The mutations include replacing the first six amino acids of the CH2 domain in IgG4 (APEFLG, SEQ ID NO: 52) with the first five amino acids of IgG2 (APPVA, SEQ ID NO: 53). TM is a CD28 transmembrane domain. tCD19 represents truncated CD19 lacking intracellular signaling. CD3 and 4-1BB are internal signaling domains of these proteins; T2A represents a cleaving peptide.

FIGS. 11A, 11B. (11A) Human CD33^{PAN} antibody clones (clones 1A9, 1H10, 1B9, 1E6, and 1D2) and (11B) human CD33^{V-set} antibody clones (clones 2E3, 2D3, and 1H8) were tested flow cytometrically against CD33+ parental ML-1 cells and ML-1 cells with CRISPR/Cas9-mediated deletion of CD33 (“CD33 KO”) as well as against REH sublines engineered to express CD33^{FL} or CD33^{ΔE2}, as indicated. A control without primary antibody was included.

FIGS. 12A, 12B. (12A) A kinetic profile was established from purified anti-CD33 antibodies by use of surface plasmon resonance technology (SPR) from the Carterra instrument. SPR is a great way to estimate the kinetic rate constants of binding interactions, of which can fit to a 1:1 Langmuir binding model to determine the on-rate (ka) and off-rate (kd). Both of these rate parameters allow for the calculation of the dissociation rate constant (kD), referred to as binding affinity. The antibody clones were captured as an array on a protein A/G lawn, which was immobilized to a HC30M chip. The first kinetic experiment used a full-length CD33 (CD33^{FL}) antigen which started at a concentration of 2 μM preceding a 4-fold titration to 2 nM. Following 10 HBSTE buffer blanks, 6 injections from low to high concentrations were subsequently flowed over the array to assess kinetics of each clone printed to the array, 1-min baseline, 5-min association, 10-min dissociation. The chip was then regenerated with 0.85% phosphoric acid pH 1.7 for a fresh reprint of the same array of clones to the protein A/G lawn, in case any lingering antigen stayed bound to the array preventing an interaction with the 2^{nd} antigen of interest, of which CD33^{ΔE2} flowed over the array of antibodies. Carterra kinetic software was used to process the data to fit the raw data to kinetic curves for each concentration of antigen injected over the array. (12B) SPR assessment of purified ECDs from CD33^{FL} or CD33^{ΔE2} binding to captured 1H10 and 2D3. Experiments were performed at 25° C. on a Biacore T100 instrument with a Series S CM4 chip. Protein A/G at 60 μg/mL in 10 mM sodium acetate, pH 4.0 was immobilized on 2 flow cells (1000 RUs) using standard amine-coupling chemistry. Capture kinetic experiments were run in 10 mM HEPES, pH 7.4, 150 mM NaCl, 3 mM EDTA, 0.05% surfactant P20 and 0.1 mg/mL IgG free bovine serum albumin buffer. Anti-human CD33 antibodies at 0.5 μg/mL were injected at 10 μL/min over the second flow cell of immobilized Protein A/G for 30-40 s to capture 40 to 58 RUs of antibody for the CD33^{FL} binding experiments, or 45-80 s to capture 75 to 95 RUs of antibody for the CD33^{ΔE2} binding experiments. Purified ectodomains for CD33^{FL} and CD33^{ΔE2} were run as concentration series at 50 μL/min over both the captured antibody and Protein A/G alone (reference) surfaces. CD33^{FL} series started at a high concentration of 160 nM for 2D3 and 1H10; CD33^{ΔE2} series started at 40 nM for 1H10 and 300 nM for 2D3. CD33 was injected for 7 minutes and allowed to dissociate for 20 or 30 minutes for most pairs. Serial 2-fold dilutions of ectodomain concentrations were run in duplicate, randomized, and included a buffer blank every 4^{th} injection. The CM4 chip was regenerated with 2 30 s injections of 0.85% H₃PO₄ at 50 μL/min and antibody recaptured prior to each CD33 injection. Data was double referenced and analyzed in BiaEval 2.0.4 software with a 1:1 binding model using local Rmax.

FIG. 13. Internalization of 1E6 and P67.6. AML cell lines were incubated with CD33 antibody at 37° C. for the time indicated. Fluorescently labeled secondary antibody was then added to quantify remaining CD33 antibody on the cell surface. Results are presented as a percentage of the fluorescence signal present at time 0.

FIG. 14. Binding of recombinant fully human CD33^{V-set} antibody 1H8 with human IgG1 framework to REH cells (human acute lymphoblastic leukemia cell line, endogenously CD33^{neg}) engineered to express human CD33^{FL}.

FIGS. 15A-15O. CAR-T cells expressing humanized (1E6, 1A9 and 1H10) scFVs directed against CD33 can be expanded in vitro with cytokines. CD8+ CAR-T cells expressing intermediate spacer CAR and 1E6, 1A9 or 1H10 scFv were sorted by fluorescence-activated cell sorting (FACS) seven days following CD3/CD28 bead stimulation and transduction. Cells were then expanded in IL-2 (50 ng/mL, 15A, 15D, 15G, 15J, and 15M); IL-7 and IL-15 (10 ng/mL each, 15B, 15E, 15H, 15K, and 15N); or IL-7, IL-15 and IL-21 (10 ng/mL each, 15C, 15F, 15I, 15L, and 15O). Media and cytokines were refreshed every other day. Cells were assessed every two days for cell number (15A-15F), cell growth (15G-15I), viability by propidium iodide exclusion (15J-15L) and diameter (15M-15O).

FIGS. 16A-16C. 1E6, 1A9 and 1H10 CD33-directed CAR-T cells with an intermediate spacer show antigen specific lysis in vitro against multiple AML cell lines. Chromium⁵¹ (Cr⁵¹)-labeled KG1a and ML-1 cells either expressing endogenous (Par) or deficient for CD33 by CRISPR-Cas9 genetic deletion (KO) were exposed for four hours to (16A) CD33-directed CD8+ CAR-T cells expanded in IL-2 (50 ng/mL), (16B) IL-7 & IL-15 (10 ng/mL each), or (16C) IL-7, IL-15 & IL-21 (10 ng/mL each) at various effector:target ratios. Supernatant was harvested and analyzed for Cr⁵¹ concentration by scintillation.

FIGS. 17A-17I. CD33-directed CD8+ CAR-T cells expressing 1E6, 1A9 or 1H10 scFvs were expanded in IL-2 (50 ng/mL, 17A, 17D, and 17G), IL-7 & 15 (10 ng/mL each, 17B, 17E, and 17H), or IL-7, IL-15 & IL-21 (10 ng/mL each, 17C, 17F, and 17I) and then exposed: to irradiated AML cell lines (ML-1 or KG1a) replete with endogenous CD33 expression (Par); cell lines deficient in CD33 by CRISPR-Cas9 genetic deletion (KO); media; or phorbol-12 myristate and ionomycin (PMA-Iono). Cell supernatant was then harvested 24 hours later and analyzed for levels of interferon-gamma (IFNγ, 17A-17C), interleukin-2 (IL-2, 17D-17F) or tumor necrosis factor alpha (TNFa, 17G-17I) by enzyme-linked immunosorbent assay (ELISA).

FIGS. 18A-18F. CD33-directed CAR-T cells expanded in IL-7, IL-15 & IL-21 show superior proliferation in vitro. CD8+ CAR-T cells expressing 1E6, 1A9 or 1H10 directed scFvs and expanded in either IL-2 (50 ng/ml, 18A and 18D), IL-7 and 15 (10 ng/ml, 18B and 18E), or IL-7, 15 & 21 (10 ng/ml, 18C and 18F) were labeled with CFSE and exposed to ML-1 cells expressing endogenous CD33 (Par) or deficient for CD33 by CRISPR-Cas9 genetic deletion (KO) or media alone for five days. Cells were then analyzed for CFSE level by flow cytometry. Summary of percent divided cells (18D-18F) with undivided peak isolated by proliferation modeling in FlowJo software.

FIG. 19. Sequences supporting the disclosure: 1H10 VH-VL scFv coding sequence (SEQ ID NO: 2); 1A9 VH-VL scFv coding sequence (SEQ ID NO: 3); 1E6 VH-VL scFv coding sequence (SEQ ID NO: 4); 1D2 VH-VL scFv coding sequence (SEQ ID NO: 5); 1B9 VH-VL scFv coding sequence (SEQ ID NO: 6); 1H8 VH-VL scFv coding sequence (SEQ ID NO: 7); 2D3 VH-VL scFv coding sequence (SEQ ID NO: 8); 2E3 VH-VL scFv coding sequence (SEQ ID NO: 9); Signal peptide coding sequence (SEQ ID NO: 188); G4Sx3 linker coding sequence (SEQ ID NO: 189); IgK signal peptide (SEQ ID NO: 158); 1H10 scFv VH-VL orientation (SEQ ID NO: 190); 1H10 scFv VL-VH orientation (SEQ ID NO: 191); 1A9 scFv VH-VL orientation (SEQ ID NO: 192); 1A9 scFv VL-VH orientation (SEQ ID NO: 193); 1E6 scFv VH-VL orientation (SEQ ID NO: 194); 1E6 scFv VL-VH orientation (SEQ ID NO: 195); 2D3 scFv VH-VL orientation (SEQ ID NO: 196); 2D3 scFv VL-VH orientation (SEQ ID NO: 197); human CD33 full length DNA coding (SEQ ID NO: 198); human CD33 full length protein (SEQ ID NO: 199); IgG4 hinge coding sequence-A (SEQ ID NO: 10); IgG4 hinge coding sequence-B (SEQ ID NO: 11); IgG4-int(DS) coding sequence (SEQ ID NO: 12); IgG4-long coding sequence (SEQ ID NO: 13); CD3ζ coding sequence (SEQ ID NO: 14); CD3ζ protein-A (SEQ ID NO: 15); CD3ζ protein-B (SEQ ID NO: 16); 4-1BB signaling coding sequence-A (SEQ ID NO: 17); 4-1BB signaling coding sequence-B (SEQ ID NO: 18); 4-1BB protein-A (SEQ ID NO: 19); 4-1 BB protein-B (SEQ ID NO: 20); CD28TM coding sequence-A (SEQ ID NO: 21); CD28TM coding sequence-B (SEQ ID NO: 22); CD28TM coding sequence-C(SEQ ID NO: 23); CD28TM protein-A (SEQ ID NO: 24); CD28TM protein-B (SEQ ID NO: 25); tCD19 coding sequence (SEQ ID NO: 26); T2A coding sequence (SEQ ID NO: 27); Thoseaasigna Virus 2A (T2A) Peptide (SEQ ID NO: 28); Porcine Teschovirus-1 2A (P2A) Peptide (SEQ ID NO: 29); Equine Rhinitis A Virus (ERAV) 2A (E2A) Peptide (SEQ ID NO: 30); Foot-And-Mouth Disease Virus 2A (F2A) Peptide (SEQ ID NO: 31); EF1 promoter-A (SEQ ID NO: 32); EF1 promoter-B (SEQ ID NO: 33); Psi (SEQ ID NO: 34); RRE (SEQ ID NO: 35); Flap (SEQ ID NO: 36); GM-CSFR encoding sequence (SEQ ID NO: 37), WPRE (SEQ ID NO: 38); delU3 (SEQ ID NO: 39); R (SEQ ID NO: 40); U5 (SEQ ID NO: 41); AmpR (SEQ ID NO: 42); CoE1 origin (SEQ ID NO: 43); SV40 (SEQ ID NO: 44); CMV (SEQ ID NO: 45); Glycosylation site; 1H10-intDS-41bb-3z-T-CD19t Top Strand (SEQ ID NO: 46); 1H10-sh-41bb-3z-T-CD19t Top Strand (SEQ ID NO: 47); 1A9-intDS-41bb-3z-T-CD19t Top Strand (SEQ ID NO: 48); 1A9-sh-41bb-3z-T-CD19t Top Strand (SEQ ID NO: 49); 1E6-intDS-41bb-3z-T-CD19t Top Strand (SEQ ID NO: 50); 1E6-sh-41bb-3z-T-CD19t Top Strand (SEQ ID NO: 51); 1H10-LvHv-intDS-41bb-3z-T-CD19t Top Strand (SEQ ID NO: 200); 1A9-LvHv-intDS-41bb-3z-T-CD19t Top Strand (SEQ ID NO: 201); 1E6-LvHv-intDS-41bb-3z-T-CD19t Top Strand (SEQ ID NO: 202); 2D3-LvHv-intDS-41bb-3z-T-CD19t Top Strand (SEQ ID NO: 203); CD33:CD22 4D protein (SEQ ID NO: 204); CD33:CD22 4D nucleotides (SEQ ID NO: 205); CD33:CD22 2D protein (SEQ ID NO: 206); CD33:CD22 2D nucleotides (SEQ ID NO: 207); CD33 V-set construct (exon 3 and 4 deleted) protein (SEQ ID NO: 208); CD33 V-set construct (exon 3 and 4 deleted) nucleotides (SEQ ID NO: 209); CD33 signal peptide (SEQ ID NO: 210); CD33 signal peptide coding sequence (SEQ ID NO: 211); 6-histidine tag coding sequence (SEQ ID NO: 212); 3× glycine linker; 3× glycine linker coding sequence; CD33 ECD (SEQ ID NO: 213); CD33 ECD coding sequence (SEQ ID NO: 214); CD33 ECD lacking CD33 amino acids 140-232 (SEQ ID NO: 215); CD33 ECD lacking CD33 amino acids 140-232 coding sequence (SEQ ID NO: 216); CD33 transmembrane domain: (SEQ ID NO: 217); CD33 transmembrane domain coding sequence (SEQ ID NO: 218); CD33 intracellular domain: (SEQ ID NO: 219); CD33 intracellular domain coding sequence (SEQ ID NO: 220); Portion of CD22 ECD that contains CD22 domains defined as Ig-like C2-type 3, Ig-like C2-type 4, Ig-like C2-type 5, Ig-like C2-type 6 (SEQ ID NO: 221); Portion of CD22 ECD that contains CD22 domains defined as Ig-like C2-type 3, Ig-like C2-type 4, Ig-like C2-type 5, Ig-like C2-type 6 coding sequence (SEQ ID NO: 222); Portion of CD22 ECD that contains CD22 domains defined as Ig-like C2-type 5, Ig-like C2-type 6 (SEQ ID NO: 223); Portion of CD22 ECD that contains CD22 domains defined as Ig-like C2-type 5, Ig-like C2-type 6 coding sequence (SEQ ID NO: 224); 1H10, 1A9, 1E6, and/or 1B9 light chain signal peptide (SEQ ID NO: 225); 1D2 light chain signal peptide (SEQ ID NO: 226); 1H8 light chain signal peptide (SEQ ID NO: 227); 2D3 light chain signal peptide (SEQ ID NO: 228); 1H10 heavy chain signal peptide (SEQ ID NO: 229); 1A9 heavy chain signal peptide (SEQ ID NO: 230); 1E6 and/or 2E3 heavy chain signal peptide (SEQ ID NO: 231); 1D2 heavy chain signal peptide (SEQ ID NO: 232); 1B9 heavy chain signal peptide (SEQ ID NO: 233); 1H8 heavy chain signal peptide (SEQ ID NO: 234); 2D3 heavy chain signal peptide (SEQ ID NO: 235); My96_int_41BB_3z_TCD19 coding sequence (SEQ ID NO: 236); My96 coding sequence (SEQ ID NO: 237); V-set directed CD33/CD3 BsAb (RC1, SEQ ID NO: 254); V-set directed CD33/CD3 BsAb without leader sequence or His tag (SEQ ID NO: 255); and V-set directed CD33 scFv protein sequence (SEQ ID NO: 256) and coding sequence (SEQ ID NO: 257).

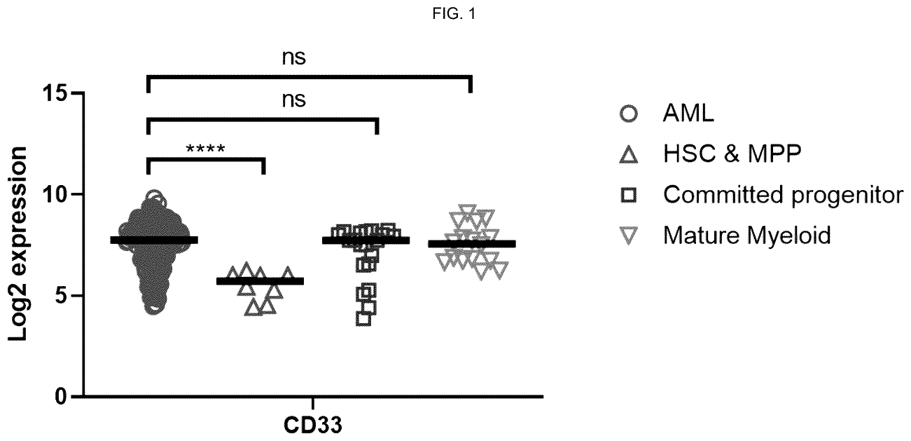

### DETAILED DESCRIPTION

According to the World Health Organization, cancer is the second leading cause of death globally, and was responsible for an estimated 9.6 million deaths in 2018. Acute myeloid leukemia (AML) is a type of cancer resulting from a malignancy of clonal, proliferative myeloid blast cells. AML is also known as acute myelocytic leukemia, acute myelogenous leukemia, acute granulocytic leukemia, and acute nonlymphocytic leukemia.

There are 20,000 new cases of AML per year in the United States (Kouchkovsky and Abdul-Hay, Blood Cancer J. 6(7):e441, 2016) and 11,000 deaths from AML each year (American Cancer Society, August 2018). Although high complete remission rates can be achieved in younger patients with AML with conventional chemotherapy at rates of 60% to 80% (Döhner, et al., 2017. Blood. 129(4): 424-447), treatment outcomes for older patients, at the age of 65 or older, remains unsatisfactory with as many as 70% of patients dying of their disease within a year of diagnosis (Meyers, et al., Appl Health Econ Health Policy, 11:275-286, 2013). Unfortunately, because of the chemo-refractoriness of leukemic stem cells, relapse after conventional therapy is common (Eppert, et al., 2011. Nat. Med. 17(9): 1086-1093) and current treatment options for relapsed/refractory (R/R) AML are dismal, resulting in less than 30% overall survival at 12 months.

For many years, the chosen treatments for cancer have been surgery, chemotherapy, and/or radiation therapy. In recent years, more targeted therapies have emerged to specifically target cancer cells by identifying and exploiting specific molecular changes seen primarily in those cells. For example, many cancer cells preferentially express particular markers on their cellular surfaces and these markers have provided targets for antibody-based therapeutics.

One key to successful targeted therapy is in the choice of the target cancer cell marker. An ideal target marker is immunogenic, plays a critical role in proliferation and differentiation, is expressed only on the surface of all malignant cells and malignant stem cells, and a large portion of patients should test positive for the marker (Cheever, et al., 2009. Clin. Cancer Res. 15(17): 5323-8337).

CD33^{FL} is primarily displayed on maturing and mature cells of the myeloid lineage, with initial expression on multipotent myeloid precursors. It is not found outside the hematopoietic system and is not thought to be expressed on pluripotent hematopoietic stem cells. Consistent with its role as a myeloid differentiation antigen, CD33^{FL} is widely expressed on malignant cells in patients with myeloid neoplasms; e.g., in AML, it is found on at least a subset of the AML blast cells in almost all cases and possibly leukemic stem cells in some. Because of this expression pattern, CD33^{FL} has been widely exploited as an antigen for targeted therapy of AML. (Walter et al., Blood 119(26):6198-6208, 2012; Cowan et al., Front. Biosci. (Landmark Ed) 18:1311-1334, 2013; Laszlo et al., Blood Ref. 28(4):143-153, 2014; and Walter, Expert Opin Investig Drugs 27(4):339-348, 2018) While unconjugated monoclonal CD33 antibodies have proved ineffective in the clinic, several recent randomized trials with the CD33 antibody-drug conjugate (ADC) gemtuzumab ozogamicin (GO) have demonstrated improved survival in subsets of patients with AML, establishing the value of antibodies in this disease and validating CD33^{FL} as the first, and so far only, therapeutic target for immunotherapy of AML (Laszlo et al., Blood Rev. 28(4):143-153, 2014; Godwin et al., Leukemia 31(9) 31(9):1855-1868, 2017). This data has validated CD33 as the first (and so far, only) target for immunotherapy in AML. In parallel to the development of new, more effective CD33-directed therapeutics (e.g. antibody-drug conjugates, radioimmunoconjugates, bispecific antibodies, chimeric antigen receptor [CAR]-modified T cells) to overcome the limitations noted with GO, interest has grown in CD33 as a drug target for other malignant and non-malignant disorders. These efforts include the targeting of CD33 splice variants not recognized by GO as well as the targeting of CD33+ tumor cells in other hematologic malignancies, CD33+ myeloid-derived suppressor cells (MDSCs) in a variety of diseases, and normal CD33+ microglial cells in Alzheimer disease (Walter, Expert Opin Biol Ther. 2020, 20(9):955-958).

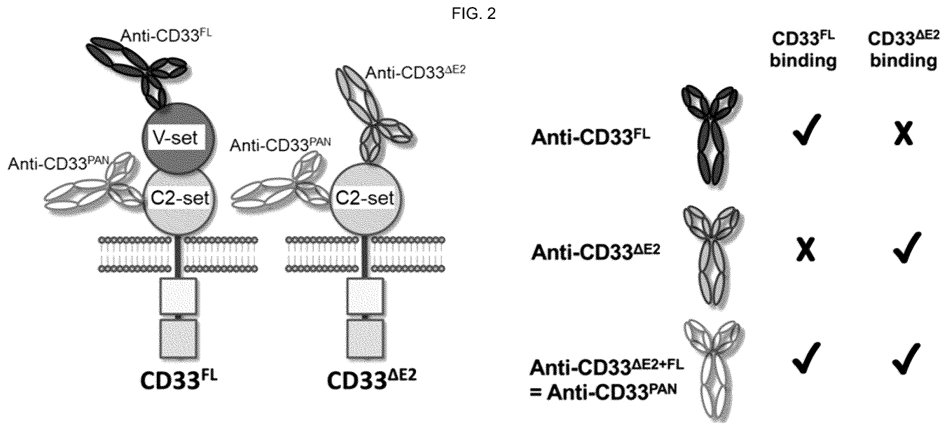

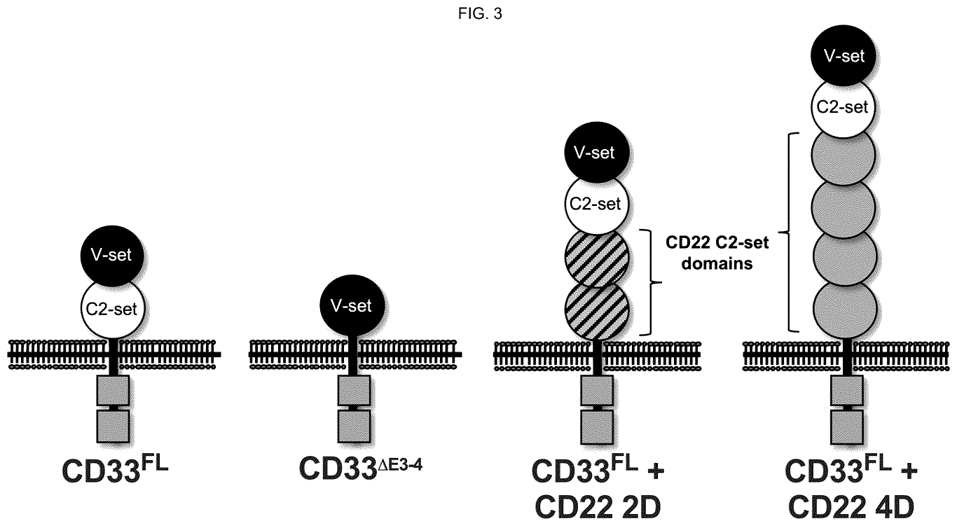

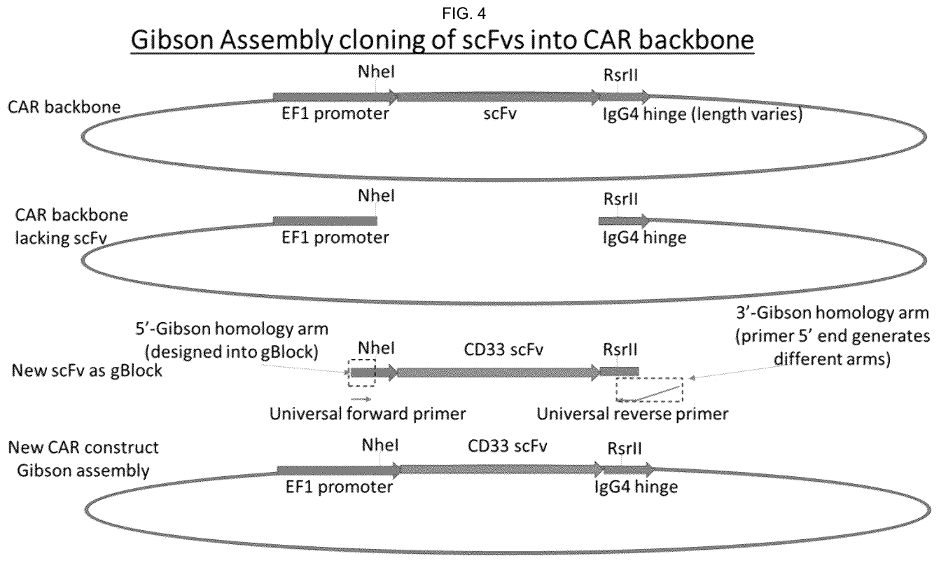

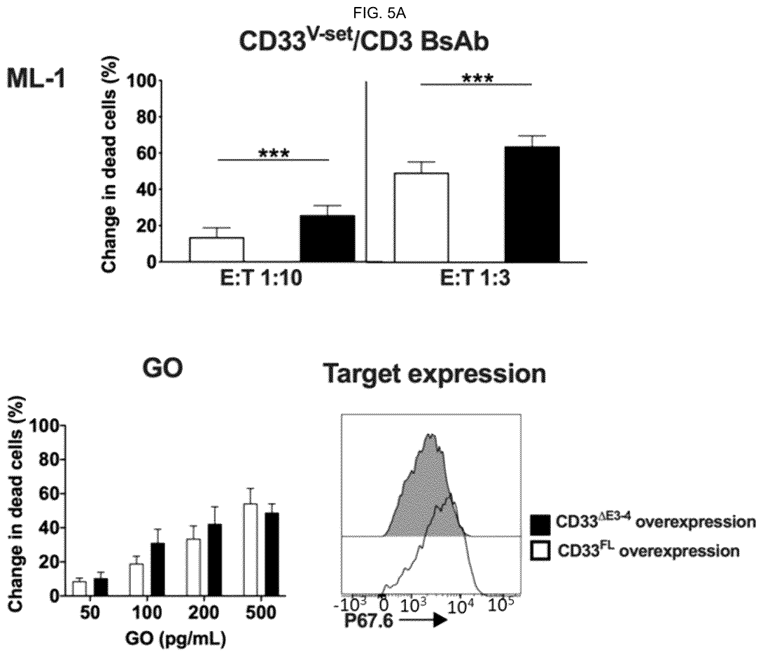

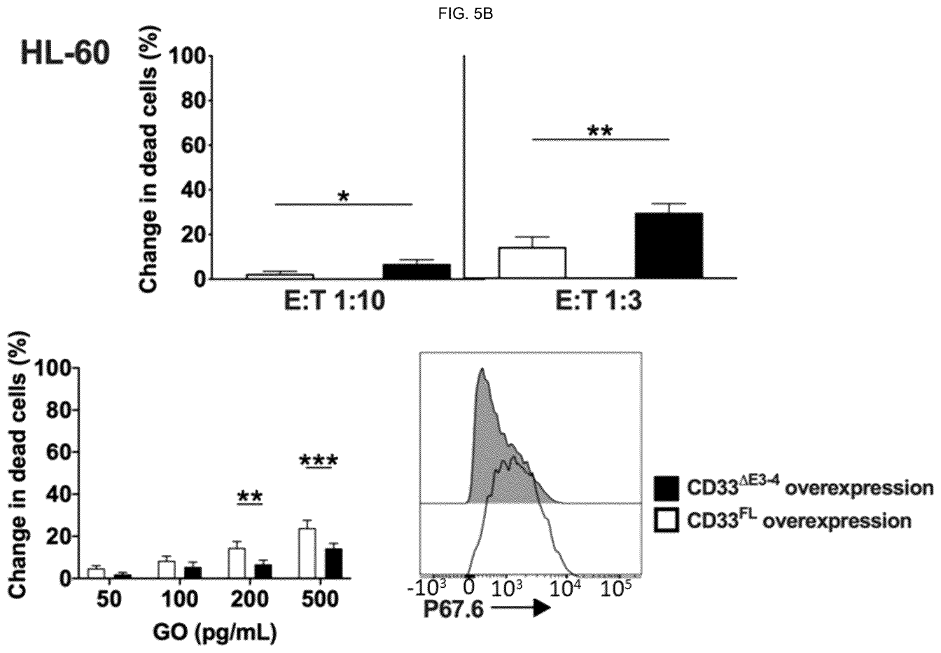

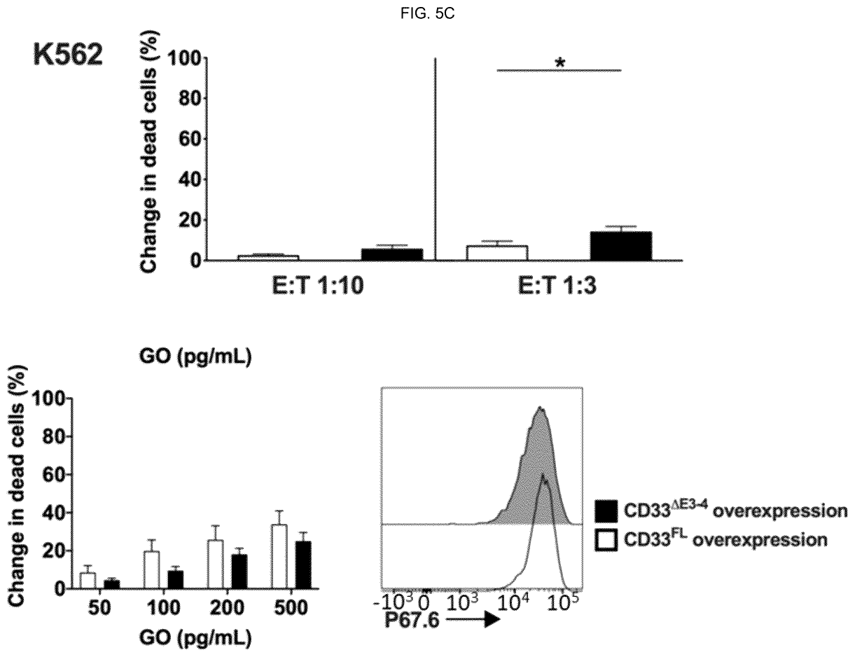

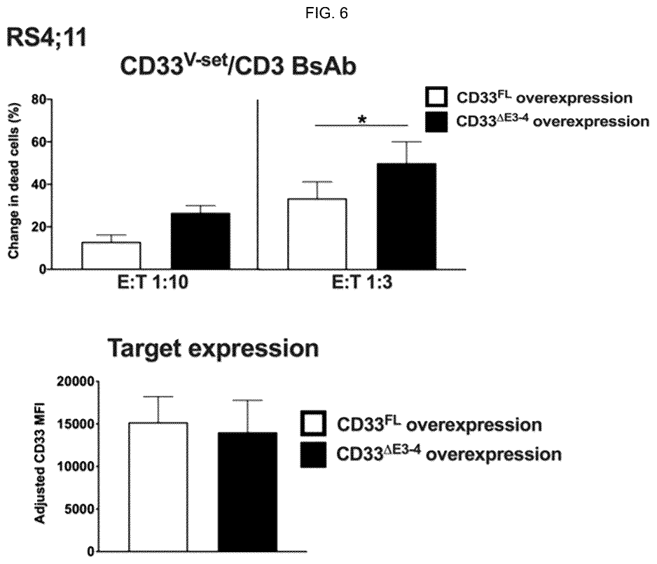

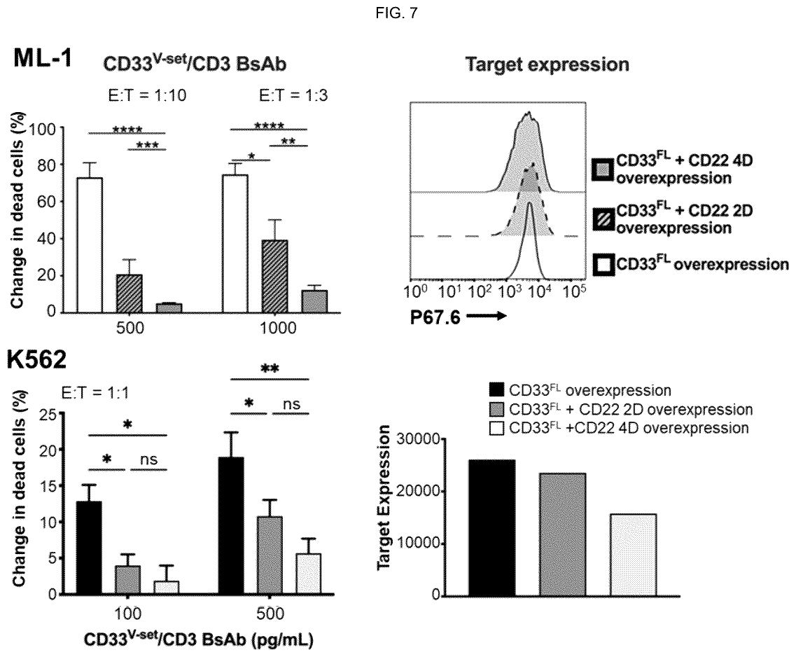

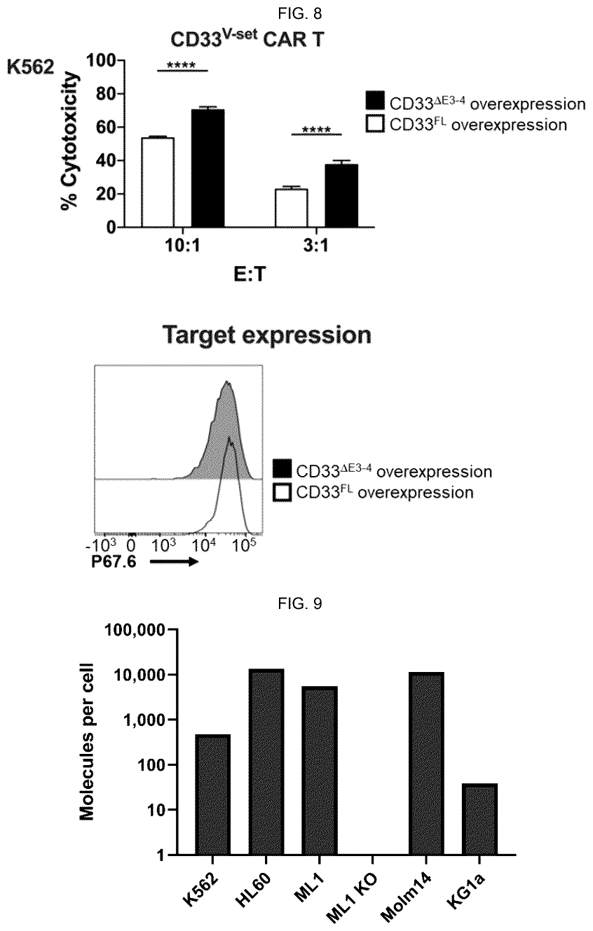

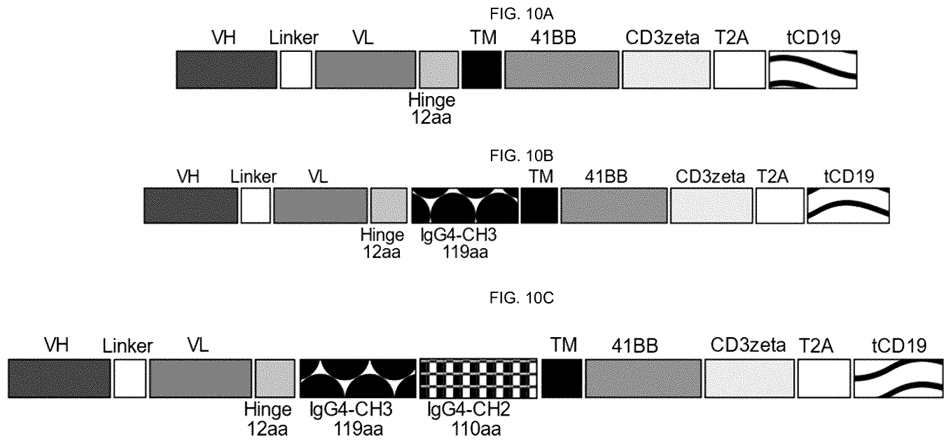

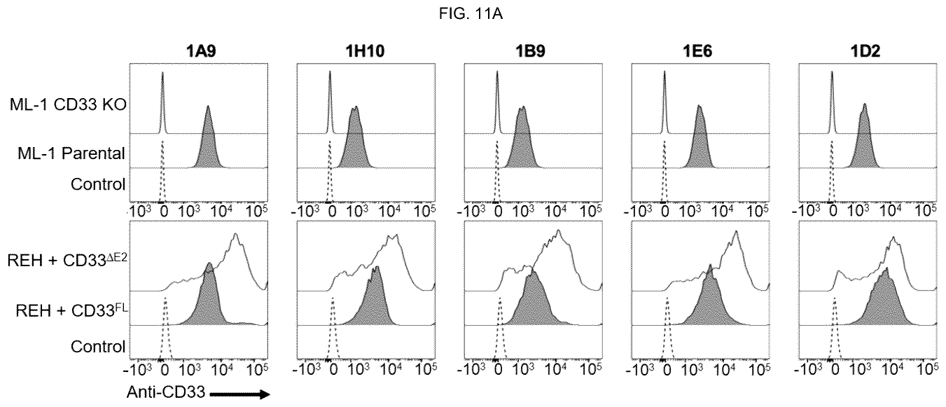

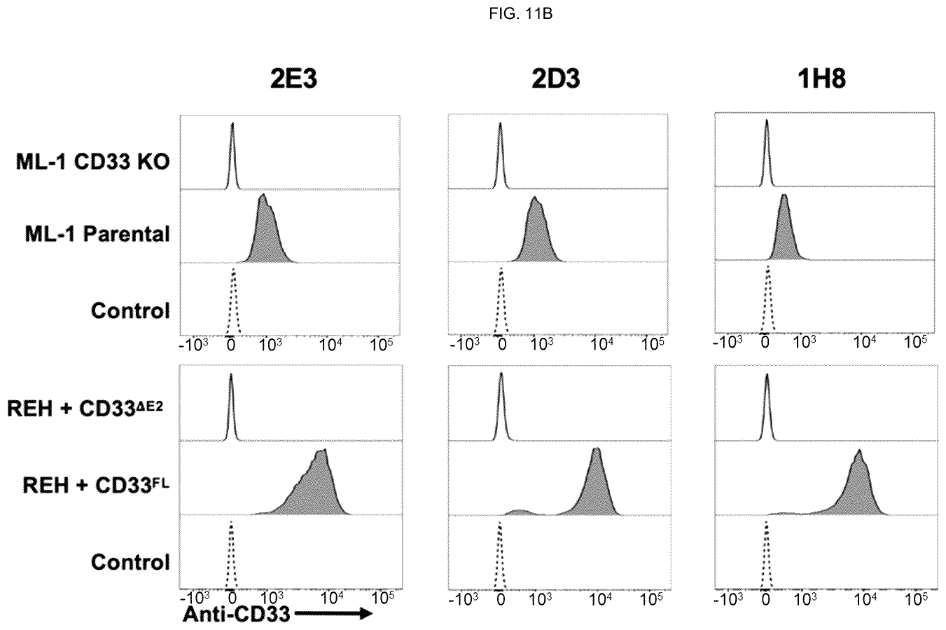

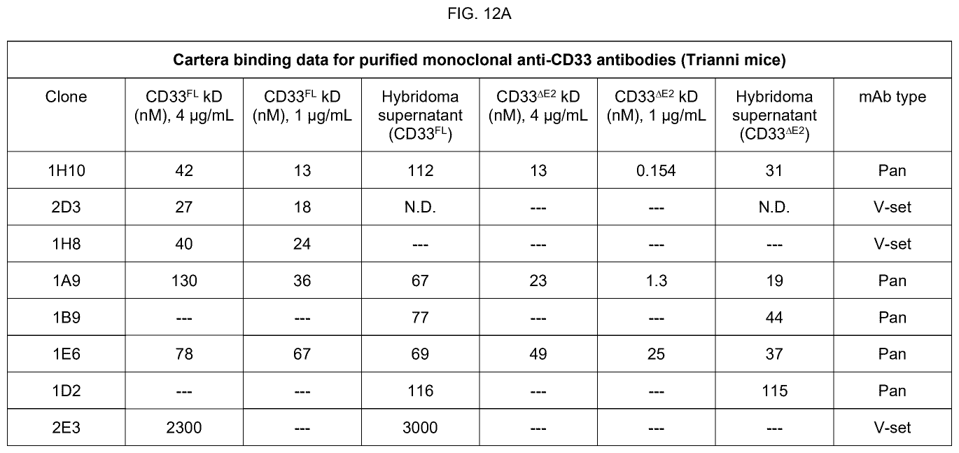

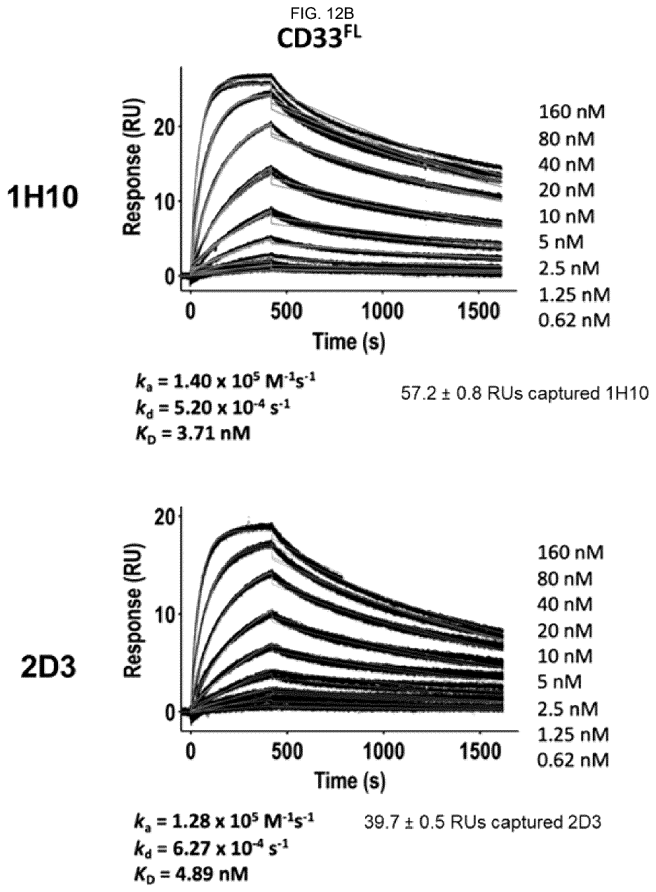

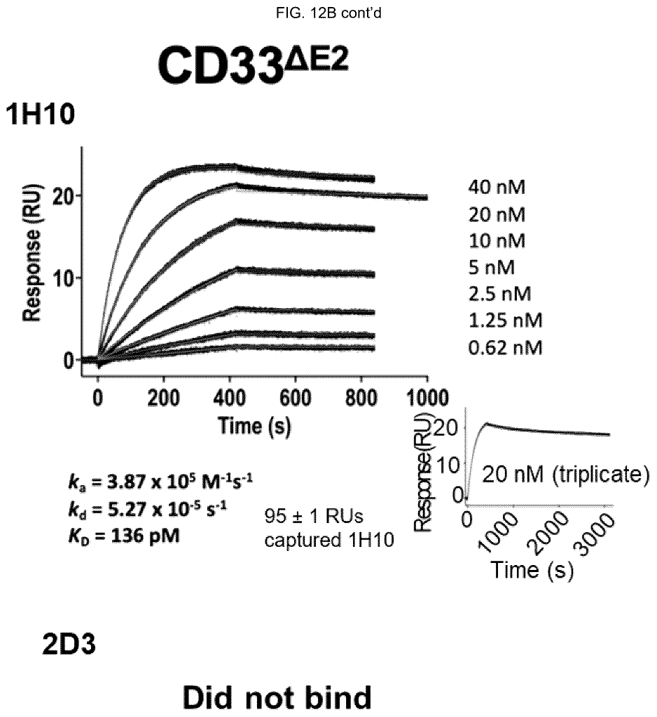

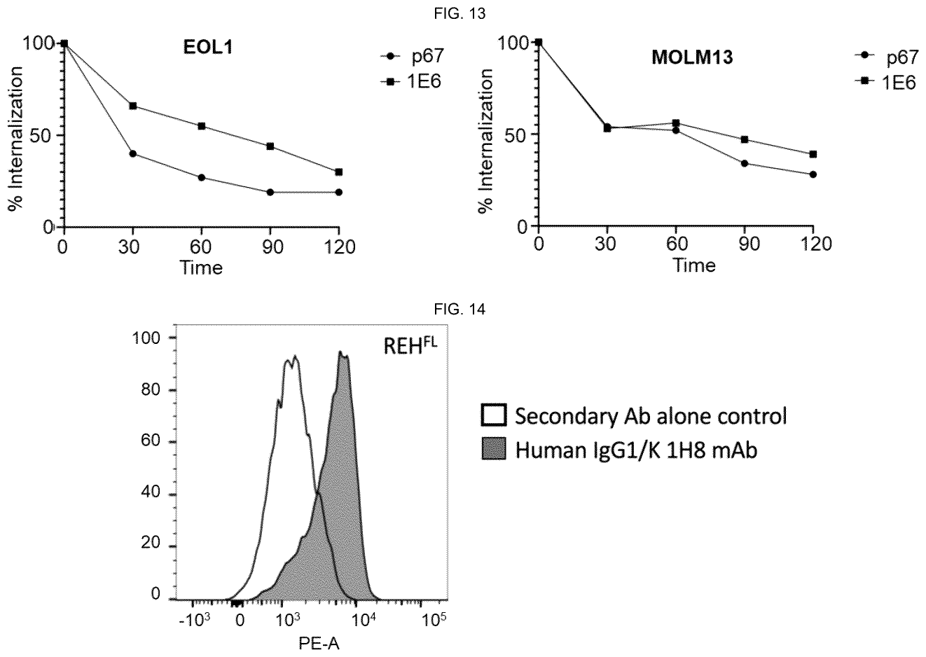

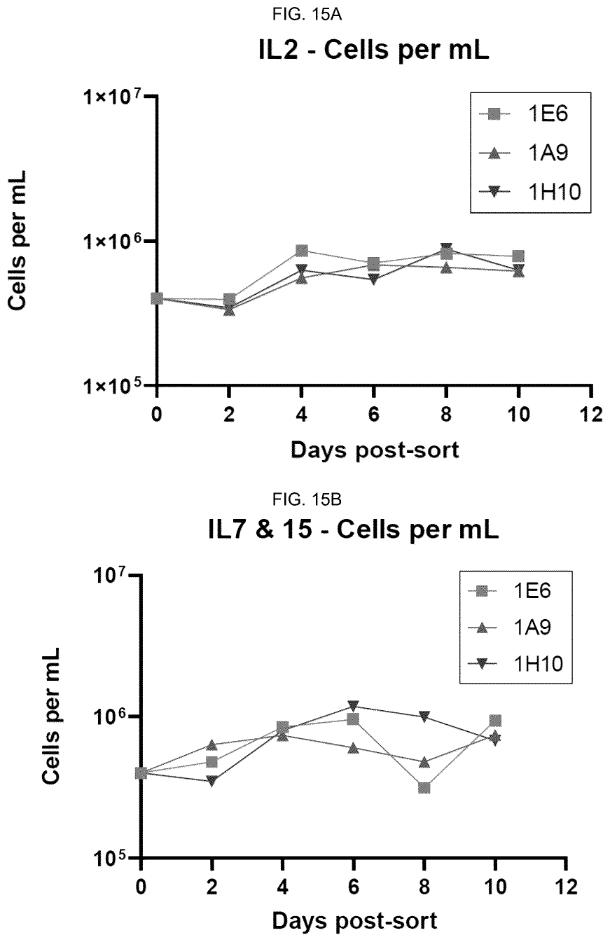

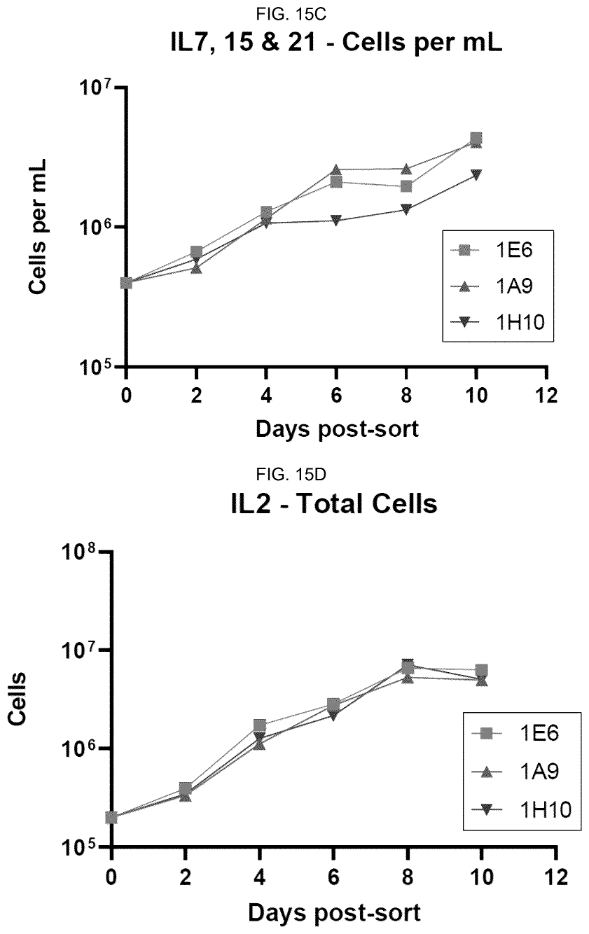

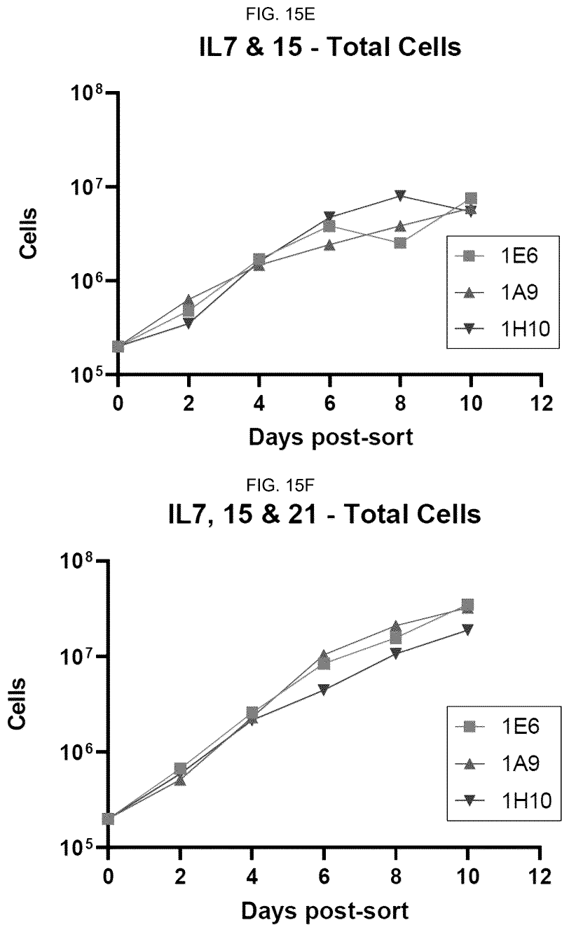

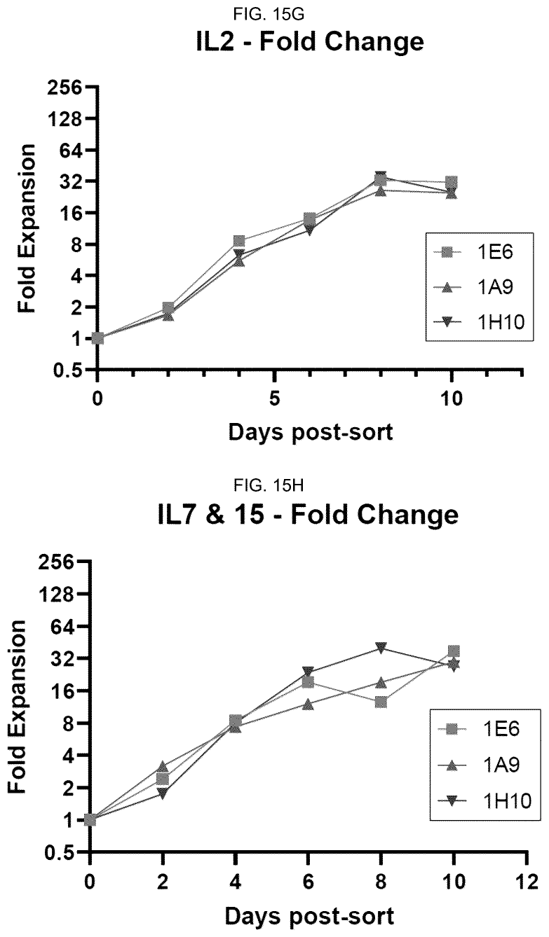

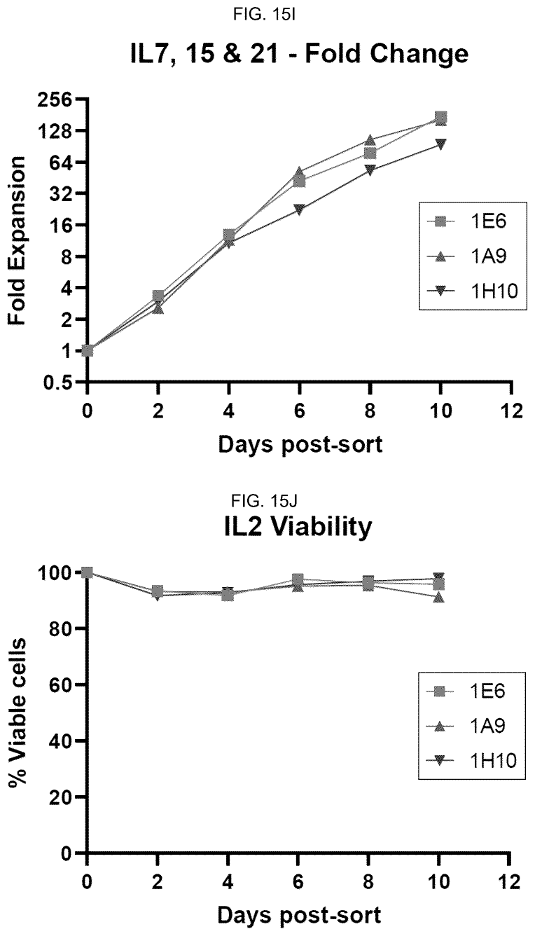

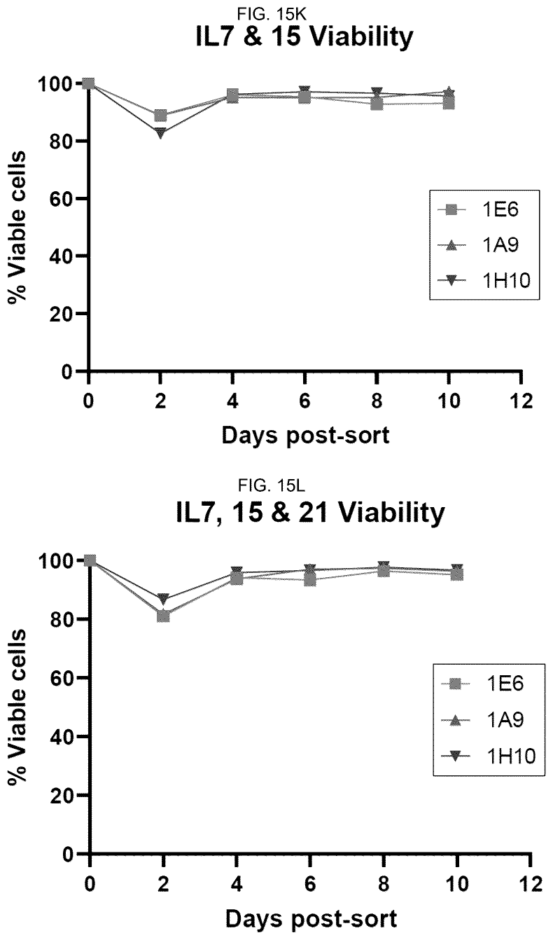

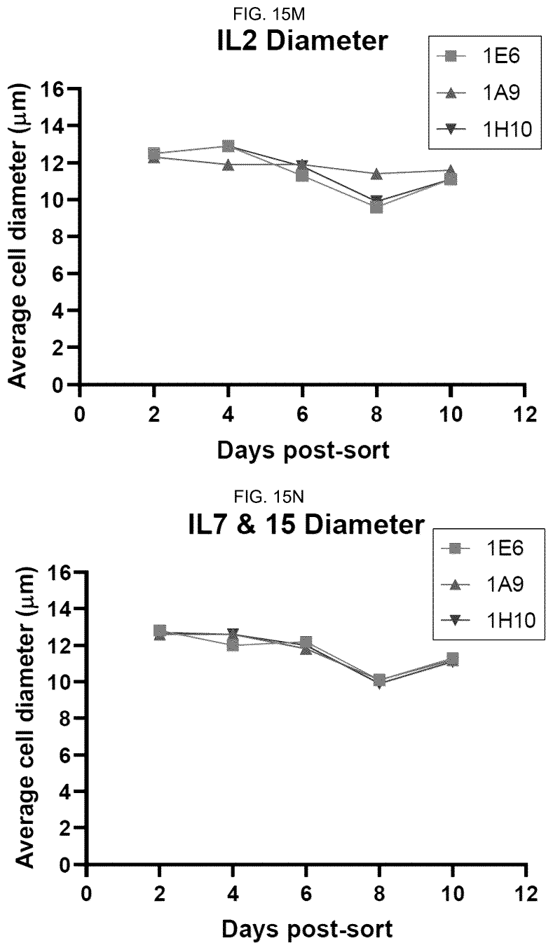

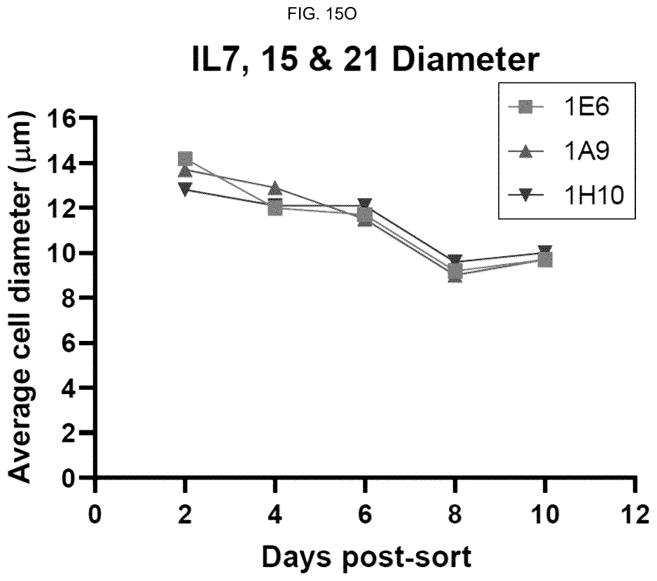

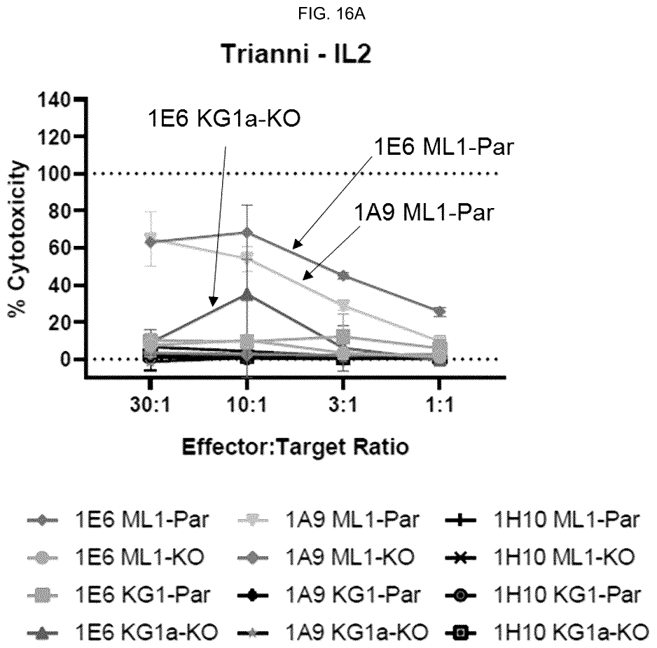

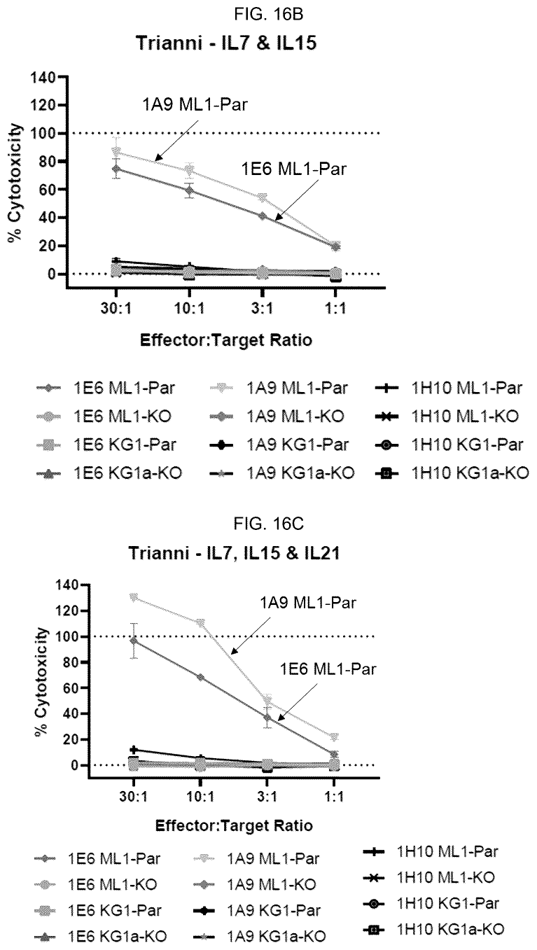

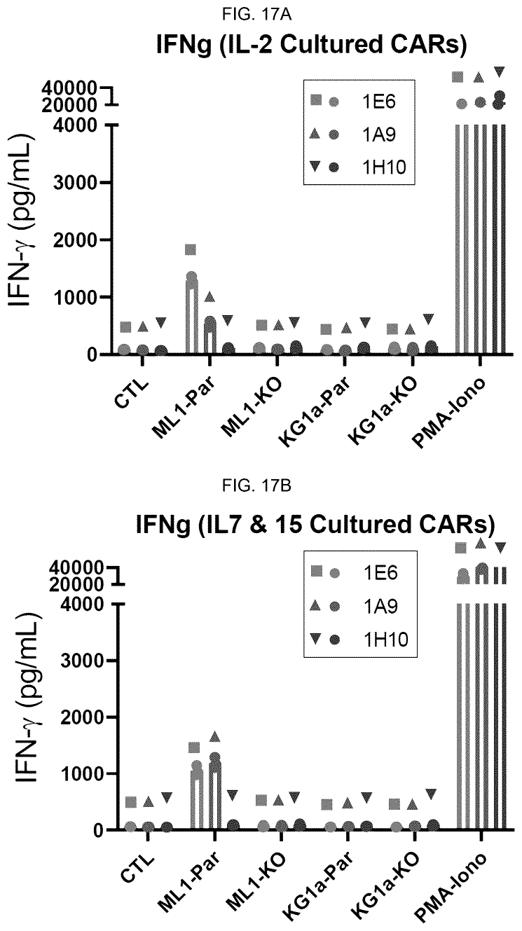

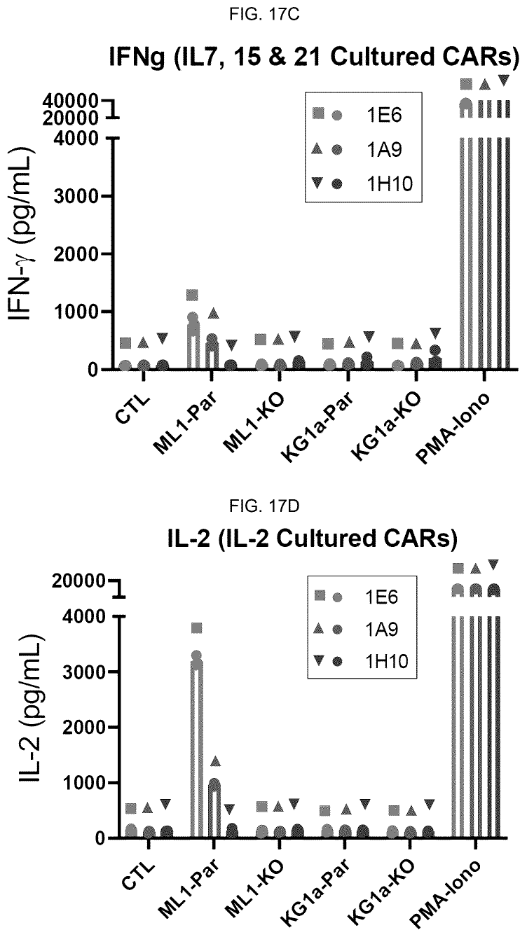

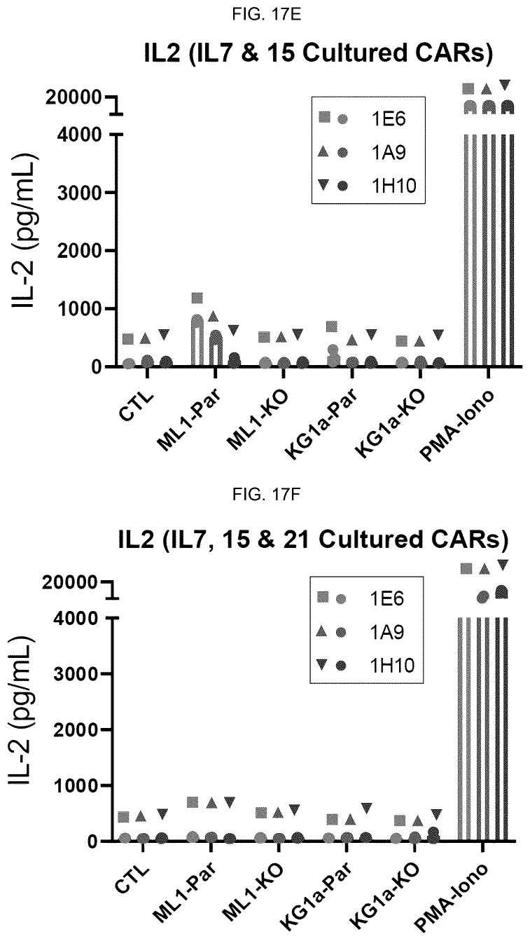

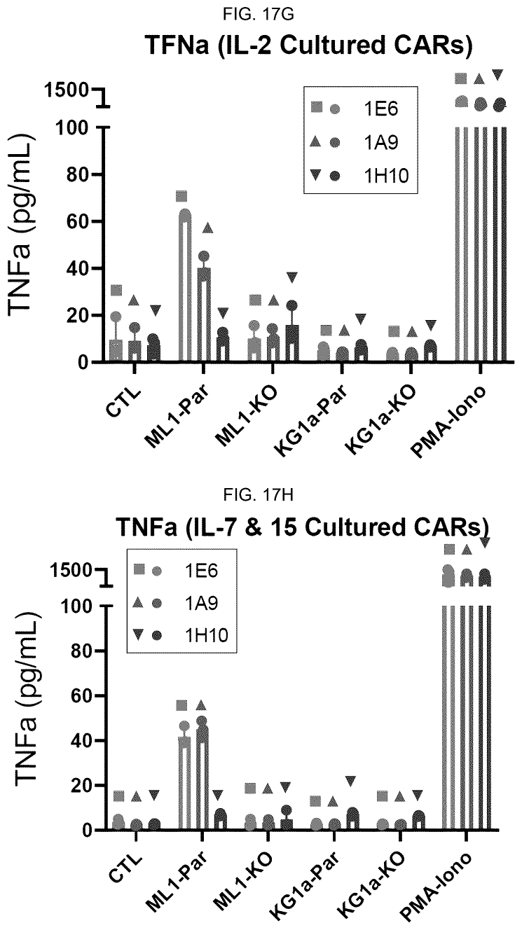

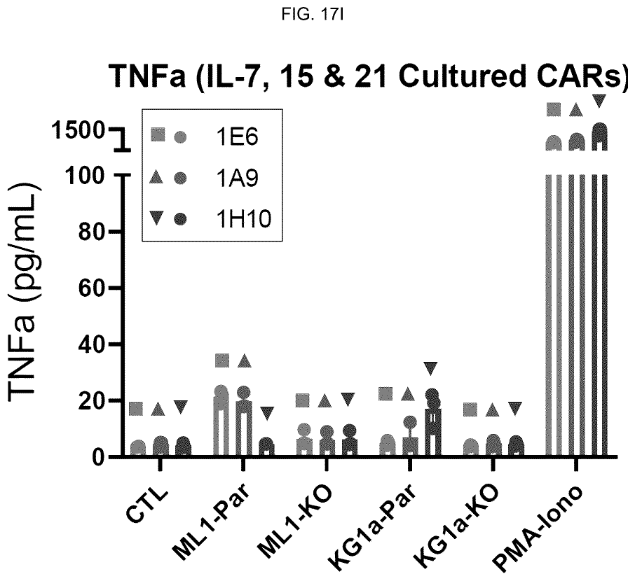

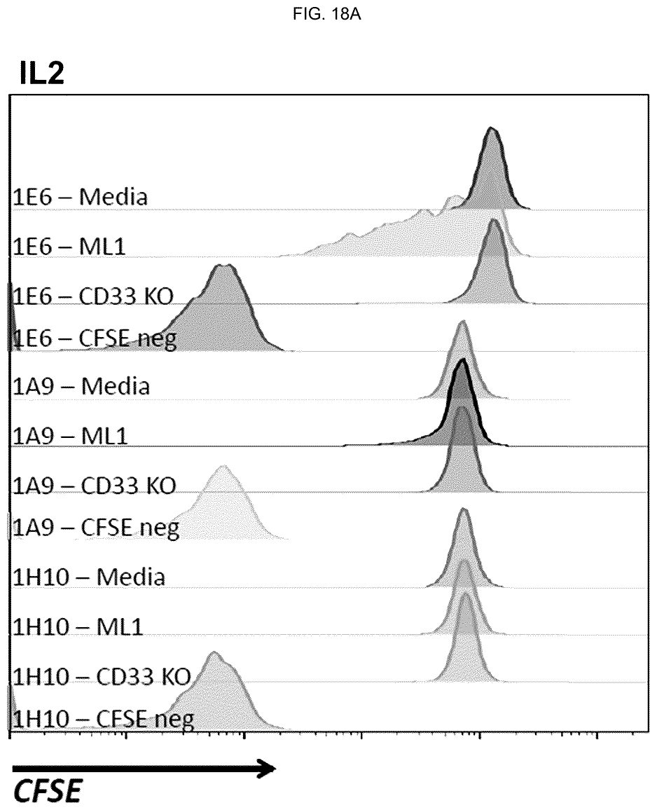

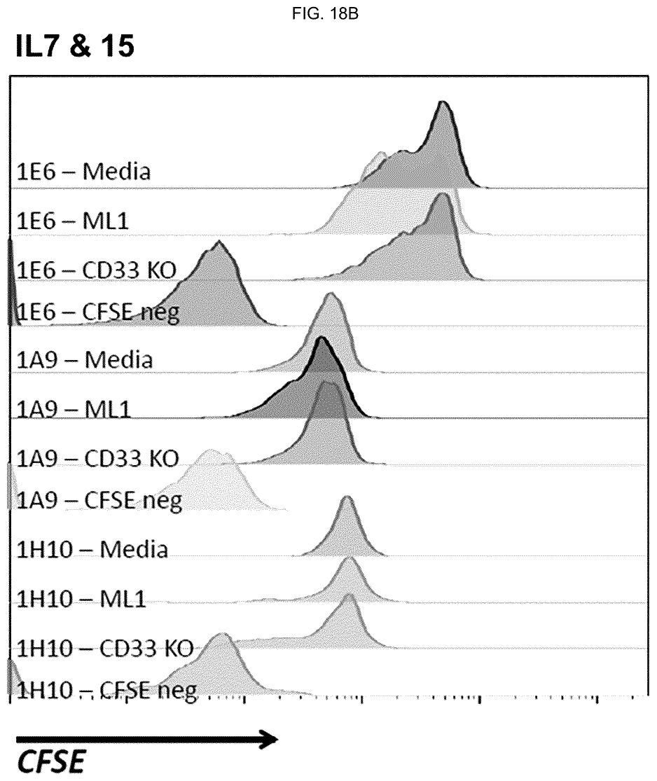

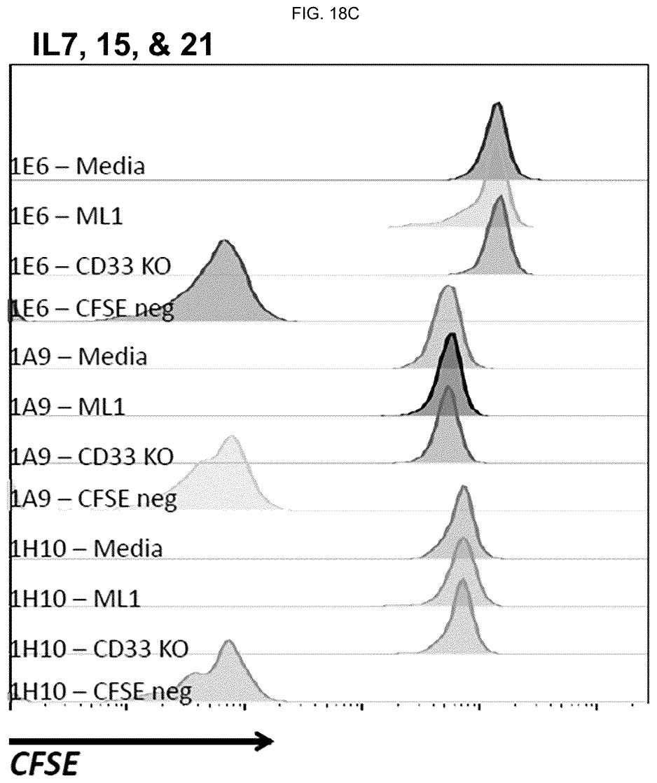

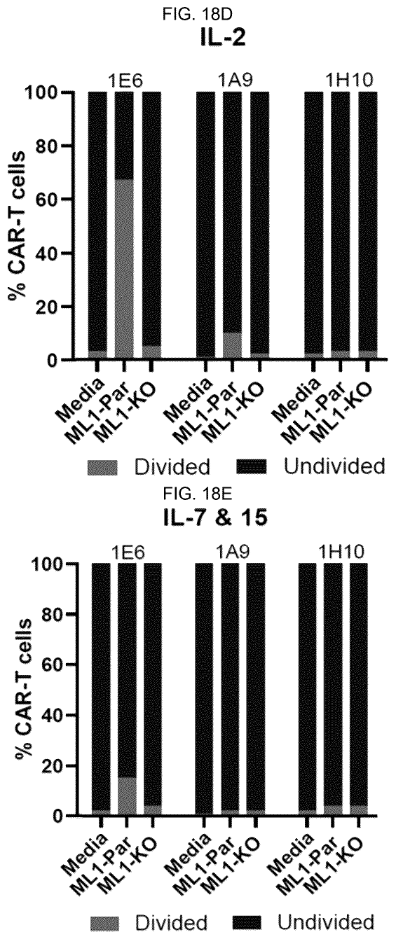

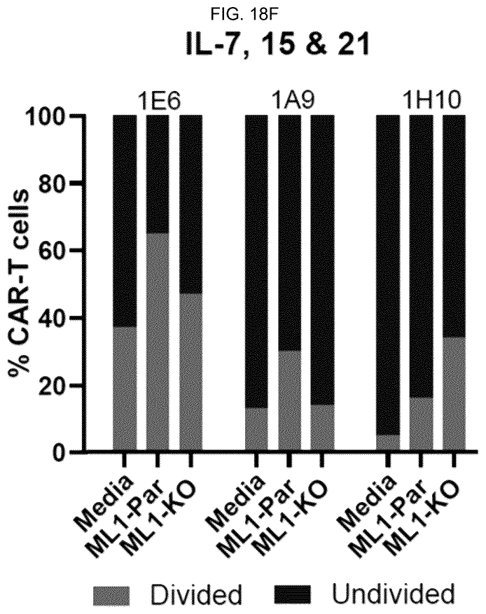

However, some patients express a truncated splice variant form of CD33 that is missing exon 2 and is referred to as CD33^{ΔE2}. CD33^{ΔE2} has been identified at the mRNA level in normal hematopoietic cells as well as leukemia cells. Regarding the latter, CD33^{ΔE2} mRNA was identified in 29 of 29 tested AML patient specimens, indicating universal expression in human AML. CD33^{ΔE2} contains the C2-set Ig-like domain but not the V-set Ig-like domain of CD33 (FIG. 2). Additional splice variants, identified at the mRNA level, include CD33^{E7}a and CD33^{ΔE2/E7a}. CD33^{E7a} uses an alternate exon 7 (E7a) which results in a truncation of the intracellular domain of CD33. CD33^{ΔE2/E7a} lacks exon 2 and also has the truncation of the intracellular domain of CD33.

Currently, however, almost all commercial diagnostic CD33 antibodies and currently clinically available CD33 antibody-based therapeutics recognize the immune-dominant V-set Ig-like domain that is encoded by exon 2 (FIG. 2). That is, CD33^{ΔE2} and other CD33 proteins that lack the V-set Ig-like domain are not recognized by almost any commercially and clinically available CD33 antibody This means that these antibodies would not recognize shorter forms of CD33 that lack the V-set domain such as CD33^{ΔE2}. This may explain the observation made in one clinical trial in pediatric AML that patients with a single nucleotide polymorphism in the CD33 gene that leads to preferential transcription of CD33^{ΔE2} and reduced translation of CD33^{FL} did not benefit from the addition of GO (which also binds to the V-set domain of CD33) to intensive chemotherapy.

Antibodies that recognize and bind the C2-set Ig-like domain of CD33 proteins regardless of the presence/absence of the V-set Ig-like domain (e.g. antibodies that bind the CD33^{ΔE2} and CD33^{FL} isoforms, referred to as CD33^{PAN} antibodies) would provide a great advance in the targeting of all CD33 isoforms, providing for broader therapeutic efficacy. These pan-binding antibodies would also provide an advance because they bind closer to the cell membrane (FIG. 2). For several therapeutic targets, the specifics of the targeted epitope have been shown to be critically important for antibody-based therapy, with membrane-proximal epitopes resulting in more potent anti-tumor effects than membrane-distal ones, as shown for CD20, CD22, CD25, and EpCAM. See, for instance, Cleary et al., J Immunol. 2017; 198(10):3999-4011; Lin, Pharmgenomics Pers Med. 2010; 3:51-59; Haso et al., Blood. 2013; 121(7):1165-1174; and Bluemel et al., Cancer Immunol Immunother. 2010; 59(8):1197-1209.

Beyond antibody-based therapeutics, significant progress has been made in genetically engineering T cells of the immune system to target and kill unwanted cell types, such as cancer cells. Many of these T cells have been genetically engineered to express a chimeric antigen receptor (CAR). CARs are proteins including several distinct subcomponents that allow the genetically modified T cells to recognize and kill cancer cells. The subcomponents include at least an extracellular component and an intracellular component. The extracellular component includes a binding domain that specifically binds a marker that is preferentially present on the surface of unwanted cells (e.g., CD33). The binding domain is typically a single-chain variable fragment (scFv) derived from a monoclonal antibody (mAb), but it can be based on other formats which include an antibody-like antigen binding site.

When the binding domain binds such markers, the intracellular component signals the T cell to destroy the bound cell. The intracellular components provide such activation signals based on the inclusion of an effector domain. First generation CARs utilized the cytoplasmic domain of CD3ζ as an effector domain. Second generation CARs utilized the cytoplasmic domain of CD3ζ in combination with cluster of differentiation 28 (CD28) or 4-1BB (CD137) cytoplasmic domains, while third generation CARs have utilized the CD3ζ cytoplasmic domain in combination with the CD28 and 4-1BB cytoplasmic domains as effector domains.

CARs additionally include a transmembrane domain that can link the extracellular component to the intracellular component.

Other subcomponents that can increase a CAR's function can also be used. For example, spacer regions can provide a CAR with additional conformational flexibility, often increasing the binding domain's ability to bind the targeted cell marker. The appropriate length of a spacer region within a particular CAR can depend on numerous factors including how close or far a targeted marker is located from the surface of an unwanted cell's membrane.

When performed ex vivo, genetically modifying T cells can involve numerous cell manipulation steps, and it has been observed that different manipulation conditions can affect the cancer-cell killing properties of the cells. Thus, in designing CARs and genetically modifying cells to express them, numerous considerations must be taken into account, including: targeted cell marker; presence and/or length of spacer; and ex vivo manipulation procedures.

The current disclosure provides chimeric antigen receptors (CARs) for the treatment of CD33-related disorders, such as AML. In particular embodiments, the CARs include a binding domain that binds CD33 regardless of which CD33 variant a patient expresses (e.g. CD33^{FL} or CD33^{ΔE2}). These CD33 binding domains are referred to as “pan” binders. In particular embodiments, the pan binders bind the membrane-proximal C2-set Ig-like domain of CD33. In particular embodiments, these pan binders are derived from newly developed antibodies: 1H10, 1A9, 1E6, 1D2, and 1B9, and can include single chain variable fragments of these antibodies. As described herein, more membrane-proximal binding enhances the immune effector function of the CAR for treatment of AML and other CD33+ disorders. Additional newly developed CD33 targeting antibodies disclosed herein bind the V-set domain of CD33 and include 1H8, 2D3, and 2E3. These antibodies provide additional CAR-based therapeutic options for patients expressing CD33^{FL}.

Binding domains of antibodies for use within a treatment can be based on combinations of binding domains based on whether a subject expresses or lacks the V-set domain of CD33. For example, if a subject expresses the V-set domain, a combination therapy including one or more binding domains of 1H10, 1A9, 1E6, 1D2, and 1B9 could be selected in combination with one or more of 1H8, 2D3, and 2E3.

In particular embodiments, the current disclosure provides CARs having a short or intermediate spacer region. In particular embodiments, the short spacer region includes the hinge region of IgG4 (12 amino acids). In particular embodiments, the intermediate spacer region includes the hinge region and the CH3 domain of IgG4 (collectively, 131 amino acids). IgG4 domains utilized as spacer regions can include mutations that prevent binding to the human Fc receptor. In particular embodiments, these mutations include replacing the first six amino acids of the CH2 domain of IgG4 (APEFLG, SEQ ID NO: 52) with the first five amino acids of IgG2 (APPVA, SEQ ID NO: 53).

In particular embodiments, the current disclosure provides expanding and activating T cells genetically modified to express a CAR disclosed herein utilizing a combination of the cytokines IL-7, IL-15, and IL-21. In particular embodiments, the current disclosure provides expanding and activating T cells genetically modified to express a CAR disclosed herein utilizing a combination of cytokines including IL-2. Expansion/activation with this combination of cytokines results in increased proliferation and antigen-specific cell lysis.

Aspects of the current disclosure are now described in more supporting detail as follows: (i) Immune Cells; (ii) Cell Sample Collection and Cell Enrichment; (iii) Genetically Modifying Cell Populations to Express Chimeric Antigen Receptors (CAR); (iii-a) Genetic Engineering Techniques; (iii-b) CAR Subcomponents; (iii-b-i) Binding Domains; (iii-b-ii) Spacer Regions; (iii-b-iii) Transmembrane Domains; (iii-b-iv) Intracellular Effector Domains; (iii-b-v) Linkers; (iii-b-vi) Control Features Including Tag Cassettes, Transduction Markers, and/or Suicide Switches; (iv) Cell Activating Culture Conditions; (v) Ex Vivo Manufactured Cell Formulations; (vi) Methods of Use; (vii) Reference Levels Derived from Control Populations; (viii) Exemplary Embodiments; and (ix) Closing Paragraphs. These headings are provided for organizational purposes only and do not limit the scope or interpretation of the disclosure.

### (i) IMMUNE CELLS

The present disclosure describes cells genetically modified to express CAR. Genetically modified cells can include T-cells, B cells, natural killer (NK) cells, NK-T cells, monocytes/macrophages, lymphocytes, hematopoietic stem cells (HSCs), hematopoietic progenitor cells (HPC), and/or a mixture of HSC and HPC (i.e., HSPC). In particular embodiments, genetically modified cells include T-cells.

Several different subsets of T-cells have been discovered, each with a distinct function. For example, a majority of T-cells have a T-cell receptor (TCR) existing as a complex of several proteins. The actual T-cell receptor is composed of two separate peptide chains, which are produced from the independent T-cell receptor alpha and beta (TCRα and TCRβ) genes and are called α- and β-TCR chains.

γδ T-cells represent a small subset of T-cells that possess a distinct T-cell receptor (TCR) on their surface. In γδ T-cells, the TCR is made up of one γ-chain and one δ-chain. This group of T-cells is much less common (2% of total T-cells) than the as T-cells.

CD3 is expressed on all mature T cells. Activated T-cells express 4-1BB (CD137), CD69, and CD25. CD5 and transferrin receptor are also expressed on T-cells.

T-cells can further be classified into helper cells (CD4+ T-cells) and cytotoxic T-cells (CTLs, CD8+ T-cells), which include cytolytic T-cells. T helper cells assist other white blood cells in immunologic processes, including maturation of B cells into plasma cells and activation of cytotoxic T-cells and macrophages, among other functions. These cells are also known as CD4+ T-cells because they express the CD4 protein on their surface. Helper T-cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T-cells destroy virally infected cells and tumor cells and are also implicated in transplant rejection. These cells are also known as CD8+ T-cells because they express the CD8 glycoprotein on their surface. These cells recognize their targets by binding to antigen associated with MHC class I, which is present on the surface of nearly every cell of the body.

“Central memory” T-cells (or “TCM”) as used herein refers to an antigen experienced CTL that expresses CD62L or CCR7 and CD45RO on the surface thereof and does not express or has decreased expression of CD45RA as compared to naive cells. In particular embodiments, central memory cells are positive for expression of CD62L, CCR7, CD25, CD127, CD45RO, and CD95, and have decreased expression of CD45RA as compared to naive cells.

“Effector memory” T-cell (or “TEM”) as used herein refers to an antigen experienced T-cell that does not express or has decreased expression of CD62L on the surface thereof as compared to central memory cells and does not express or has decreased expression of CD45RA as compared to a naive cell. In particular embodiments, effector memory cells are negative for expression of CD62L and CCR7, compared to naive cells or central memory cells, and have variable expression of CD28 and CD45RA. Effector T-cells are positive for granzyme B and perforin as compared to memory or naive T-cells.

“Naive” T-cells as used herein refers to a non-antigen experienced T cell that expresses CD62L and CD45RA and does not express CD45RO as compared to central or effector memory cells. In particular embodiments, naive CD8+T lymphocytes are characterized by the expression of phenotypic markers of naive T-cells including CD62L, CCR7, CD28, CD127, and CD45RA.

Natural killer cells (also known as NK cells, K cells, and killer cells) are activated in response to interferons or macrophage-derived cytokines. They serve to contain viral infections while the adaptive immune response is generating antigen-specific cytotoxic T cells that can clear the infection. NK cells express CD8, CD16 and CD56 but do not express CD3.

NK cells include NK-T cells. NK-T cells are a specialized population of T cells that express a semi invariant T cell receptor (TCR ab) and surface antigens typically associated with natural killer cells. NK-T cells contribute to antibacterial and antiviral immune responses and promote tumor-related immunosurveillance or immunosuppression. Like natural killer cells, NK-T cells can also induce perforin-, Fas-, and TNF-related cytotoxicity. Activated NK-T cells are capable of producing IFN-γ and IL-4. In particular embodiments, NK-T cells are CD3+/CD56+.

Macrophages (and their precursors, monocytes) reside in every tissue of the body (in certain instances as microglia, Kupffer cells and osteoclasts) where they engulf apoptotic cells, pathogens and other non-self-components. Monocytes/macrophages express CD11b, F4/80; CD68; CD11c; IL-4Rα; and/or CD163.

Immature dendritic cells (i.e., pre-activation) engulf antigens and other non-self-components in the periphery and subsequently, in activated form, migrate to T-cell areas of lymphoid tissues where they provide antigen presentation to T cells. Dendritic cells express CD1a, CD1b, CD1c, CD1d, CD21, CD35, CD39, CD40, CD86, CD101, CD148, CD209, and DEC-205.

Hematopoietic Stem/Progenitor Cells or HSPC refer to a combination of hematopoietic stem cells and hematopoietic progenitor cells.

Hematopoietic stem cells refer to undifferentiated hematopoietic cells that are capable of self-renewal either in vivo, essentially unlimited propagation in vitro, and capable of differentiation to all other hematopoietic cell types.

A hematopoietic progenitor cell is a cell derived from hematopoietic stem cells or fetal tissue that is capable of further differentiation into mature cell types. In certain embodiments, hematopoietic progenitor cells are CD24^{lo} Lin⁻ CD117⁺ hematopoietic progenitor cells. HPCs can differentiate into (i) myeloid progenitor cells which ultimately give rise to monocytes and macrophages, neutrophils, basophils, eosinophils, erythrocytes, megakaryocytes/platelets, or dendritic cells; or (ii) lymphoid progenitor cells which ultimately give rise to T-cells, B-cells, and NK-cells.

HSPC can be positive for a specific marker expressed in increased levels on HSPC relative to other types of hematopoietic cells. For example, such markers include CD34, CD43, CD45RO, CD45RA, CD59, CD90, CD109, CD117, CD133, CD166, HLA DR, or a combination thereof. Also, the HSPC can be negative for an expressed marker relative to other types of hematopoietic cells. For example, such markers include Lin, CD38, or a combination thereof. Preferably, the HSPC are CD34⁺ cells.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Cells to be genetically modified according to the teachings of the current disclosure can be patient-derived cells (autologous) or allogeneic when appropriate, and can also be in vivo or ex vivo.

### (ii) CELL SAMPLE COLLECTION AND CELL ENRICHMENT

Methods of sample collection and enrichment are known by those skilled in the art. In some embodiments, cells are derived from cell lines. The cells in some embodiments are obtained from a xenogeneic source, for example, from mouse, rat, non-human primate, or pig. In particular embodiments, cells are derived from humans.

In some embodiments, T cells are derived or isolated from samples such as whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. In particular embodiments, cells from the circulating blood of a subject are obtained, e.g., by apheresis or leukapheresis. The samples, in particular embodiments, contain lymphocytes, including T cells, monocytes, granulocytes, B cells, other nucleated white blood cells, HSC, HPC, HSPC, red blood cells, and/or platelets, and in some aspects contains cells other than red blood cells and platelets and further processing is necessary.

In some embodiments, blood cells collected from a subject are washed, e.g., to remove the plasma fraction and to place the cells in an appropriate buffer or media for subsequent processing steps. In particular embodiments, the cells are washed with phosphate buffered saline (PBS). In some embodiments, the wash solution lacks calcium and/or magnesium and/or many or all divalent cations. Washing can be accomplished using a semi-automated “flow-through” centrifuge (for example, the Cobe 2991 cell processor, Baxter) according to the manufacturer's instructions. Tangential flow filtration (TFF) can also be performed. In particular embodiments, cells can be re-suspended in a variety of biocompatible buffers after washing, such as, Ca++/Mg++ free PBS.

The isolation can include one or more of various cell preparation and separation steps, including separation based on one or more properties, such as size, density, sensitivity or resistance to particular reagents, and/or affinity, e.g., immunoaffinity, to antibodies or other binding partners. In particular embodiments, the isolation is carried out using the same apparatus or equipment sequentially in a single process stream and/or simultaneously. In particular embodiments, the isolation, culture, and/or engineering of the different populations is carried out from the same starting composition or material, such as from the same sample.

In particular embodiments, a sample can be enriched for T cells by using density-based cell separation methods and related methods. For example, white blood cells can be separated from other cell types in the peripheral blood by lysing red blood cells and centrifuging the sample through a Percoll or Ficoll gradient.

In particular embodiments, a bulk T cell population can be used that has not been enriched for a particular T cell type. In particular embodiments, a selected T cell type can be enriched for and/or isolated based on cell-marker based positive and/or negative selection. In positive selection, cells having bound cellular markers are retained for further use. In negative selection, cells not bound by a capture agent, such as an antibody to a cellular marker are retained for further use. In some examples, both fractions can be retained for a further use.

The separation need not result in 100% enrichment or removal of a particular cell population or cells expressing a particular marker. For example, positive selection of or enrichment for cells of a particular type refers to increasing the number or percentage of such cells but need not result in a complete absence of cells not expressing the marker. Likewise, negative selection, removal, or depletion of cells of a particular type refers to decreasing the number or percentage of such cells but need not result in a complete removal of all such cells.

In some examples, multiple rounds of separation steps are carried out, where the positively or negatively selected fraction from one step is subjected to another separation step, such as a subsequent positive or negative selection.

In some embodiments, an antibody or binding domain for a cellular marker is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, NJ); see also U.S. Pat. Nos. 4,452,773; 4,795,698; 5,200,084; and EP 452342.

In some embodiments, affinity-based selection is via magnetic-activated cell sorting (MACS) (Miltenyi Biotec, Auburn, CA). MACS systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376). In both cases, cells can be labeled with multiple markers, allowing for the isolation of well-defined cell subsets at high purity.

Cell-markers for different T cell subpopulations are described above. In particular embodiments, specific subpopulations of T cells, such as cells positive or expressing high levels of one or more surface markers, e.g., CCR7, CD45RO, CD8, CD27, CD28, CD62L, CD127, CD4, and/or CD45RA T cells, are isolated by positive or negative selection techniques.

CD3+, CD28+ T cells can be positively selected for and expanded using anti-CD3/anti-CD28 conjugated magnetic beads (e.g., DYNABEADS® M-450 CD3/CD28 T Cell Expander).

In particular embodiments, a CD8+ or CD4+ selection step is used to separate CD4+ helper and CD8+ cytotoxic T cells. Such CD8+ and CD4+ populations can be further sorted into sub-populations by positive or negative selection for markers expressed or expressed to a relatively higher degree on one or more naive, memory, and/or effector T cell subpopulations.

In some embodiments, enrichment for central memory T (TCM) cells is carried out. In particular embodiments, memory T cells are present in both CD62L subsets of CD8+ peripheral blood lymphocytes. PBMC can be enriched for or depleted of CD62L, CD8 and/or CD62L+CD8+ fractions, such as by using anti-CD8 and anti-CD62L antibodies.

In some embodiments, the enrichment for central memory T (TCM) cells is based on positive or high surface expression of CCR7, CD45RO, CD27, CD62L, CD28, CD3, and/or CD127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CCR7, CD45RO, and/or CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or RORI, and positive selection based on a marker characteristic of central memory T cells, such as CCR7, CD45RO, and/or CD62L, where the positive and negative selections are carried out in either order.

In particular embodiments, cell enrichment results in a bulk CD8+ FACs-sorted cell population.

Other cell types can be enriched based on known marker profiles and techniques. For example, CD34+ HSC, HSP, and HSPC can be enriched using anti-CD34 antibodies directly or indirectly conjugated to magnetic particles in connection with a magnetic cell separator, for example, the CliniMACS® Cell Separation System (Miltenyi Biotec, Bergisch Gladbach, Germany).

### (iii) GENETICALLY MODIFYING CELL POPULATIONS TO EXPRESS CHIMERIC ANTIGEN RECEPTORS (CAR)

Cell populations are genetically modified to express chimeric antigen receptors (CAR) described herein.

(iii-a) Genetic Engineering Techniques. Desired genes encoding CAR disclosed herein can be introduced into cells by any method known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, etc. Numerous techniques are known in the art for the introduction of foreign genes into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the gene to the cell, so that the gene is expressible by the cell and, in certain instances, preferably heritable and expressible by its cell progeny.

The term “gene” refers to a nucleic acid sequence (used interchangeably with polynucleotide or nucleotide sequence) that encodes a CAR including a CD33-binding domain as described herein. This definition includes various sequence polymorphisms, mutations, and/or sequence variants wherein such alterations do not substantially affect the function of the encoded CAR. The term “gene” may include not only coding sequences but also regulatory regions such as promoters, enhancers, and termination regions. The term further can include all introns and other DNA sequences spliced from an mRNA transcript, along with variants resulting from alternative splice sites. Gene sequences encoding the molecule can be DNA or RNA that directs the expression of the chimeric molecule. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type. Portions of complete gene sequences are referenced throughout the disclosure as is understood by one of ordinary skill in the art.

Gene sequences encoding CAR are provided herein and can also be readily prepared by synthetic or recombinant methods from the relevant amino acid sequences and other description provided herein. In embodiments, the gene sequence encoding any of these sequences can also have one or more restriction enzyme sites at the 5′ and/or 3′ ends of the coding sequence in order to provide for easy excision and replacement of the gene sequence encoding the sequence with another gene sequence encoding a different sequence. In embodiments, the gene sequence encoding the sequences can be codon optimized for expression in mammalian cells.

“Encoding” refers to the property of specific sequences of nucleotides in a gene, such as a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system. A “gene sequence encoding a protein” includes all nucleotide sequences that are degenerate versions of each other and that code for the same amino acid sequence or amino acid sequences of substantially similar form and function.

Polynucleotide gene sequences encoding more than one portion of an expressed CAR can be operably linked to each other and relevant regulatory sequences. For example, there can be a functional linkage between a regulatory sequence and an exogenous nucleic acid sequence resulting in expression of the latter. For another example, a first nucleic acid sequence can be operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary or helpful, join coding regions, into the same reading frame.

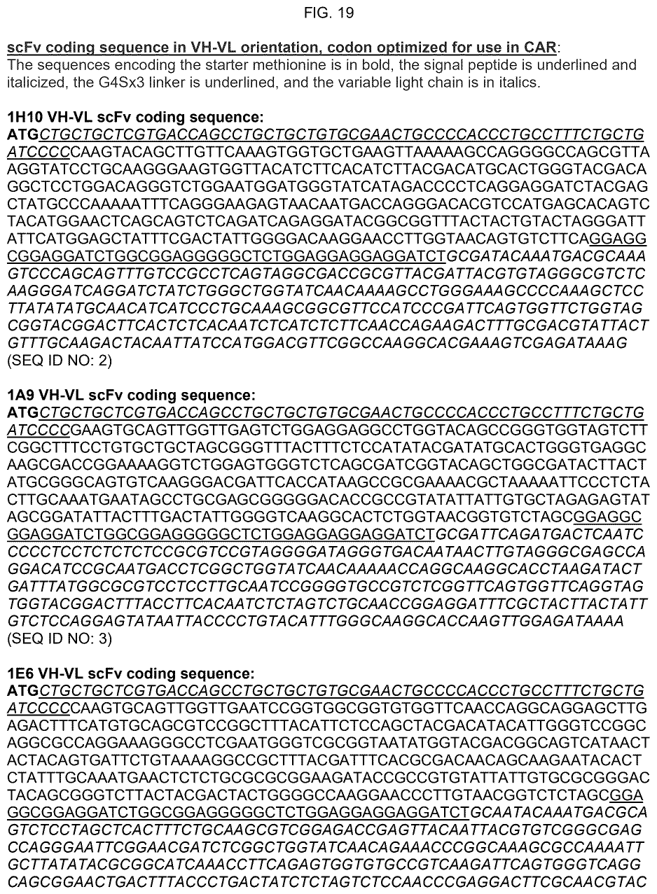

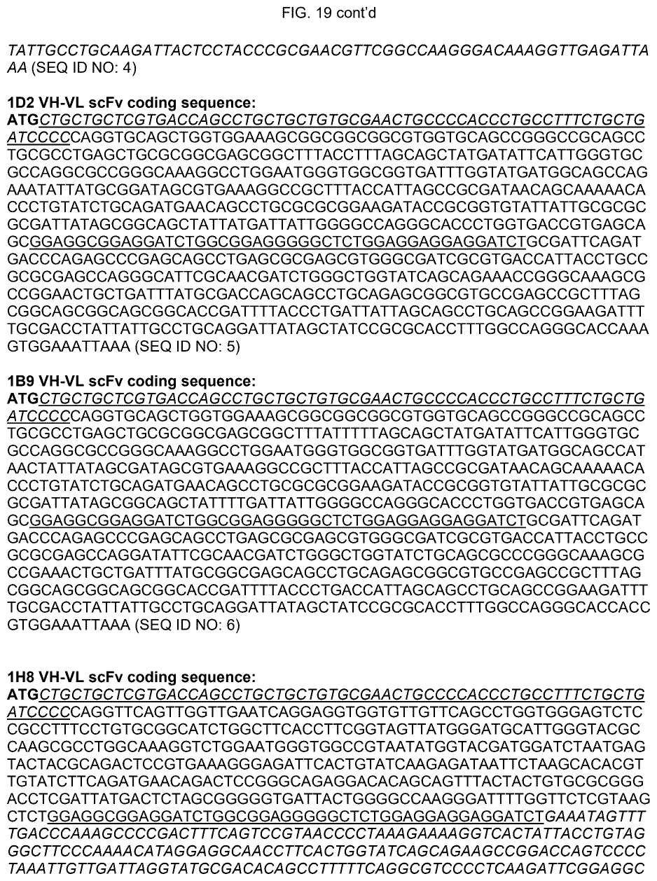

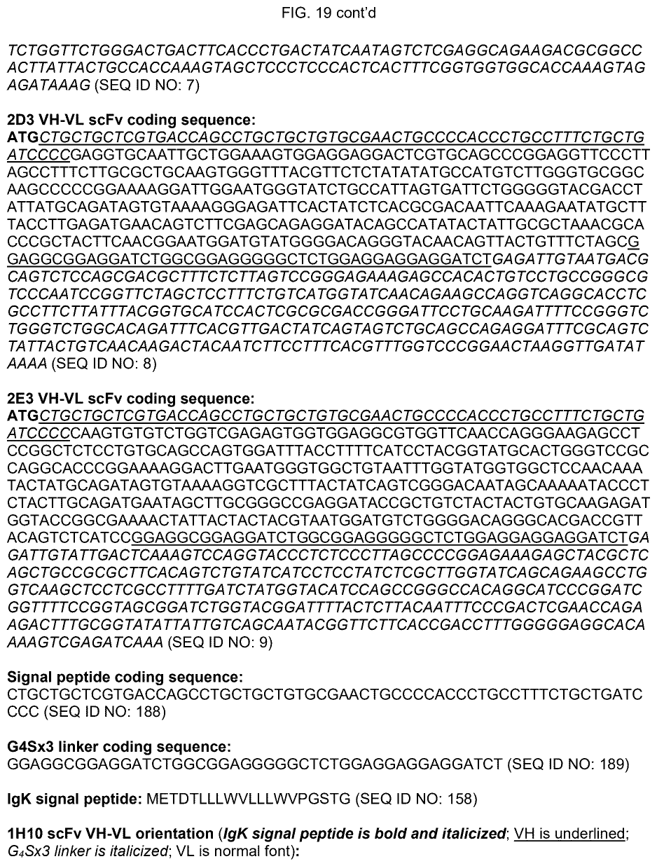

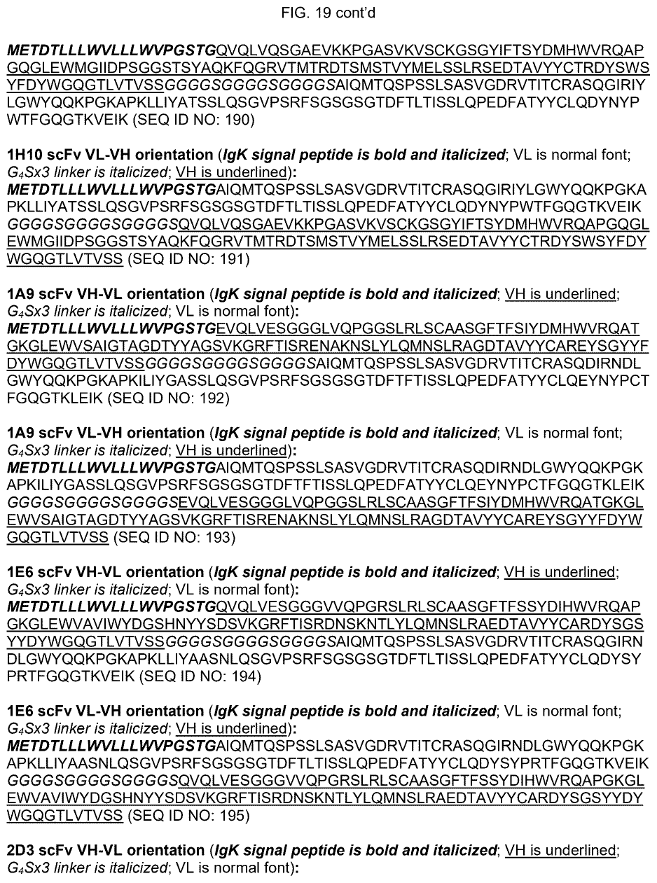

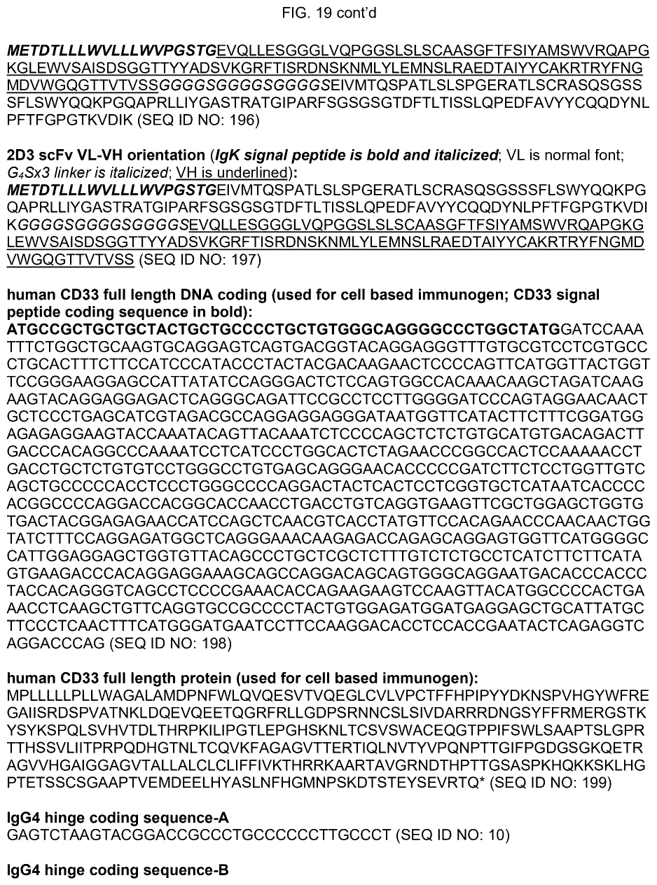

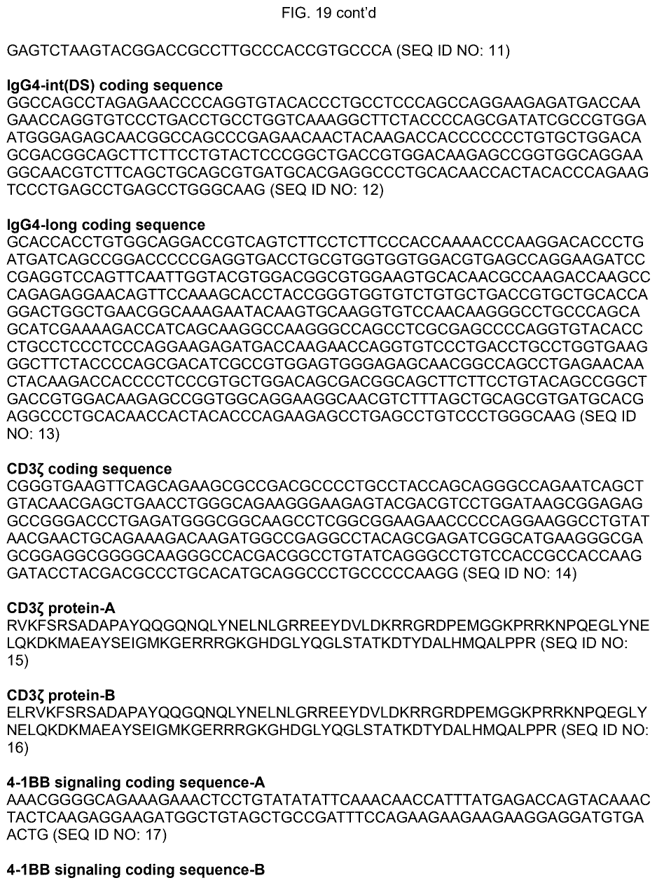

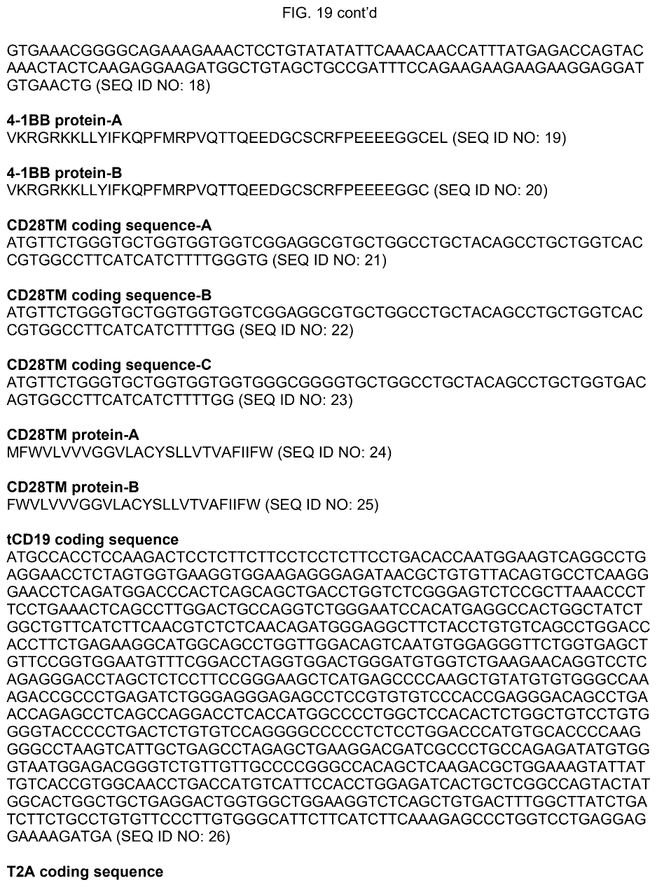

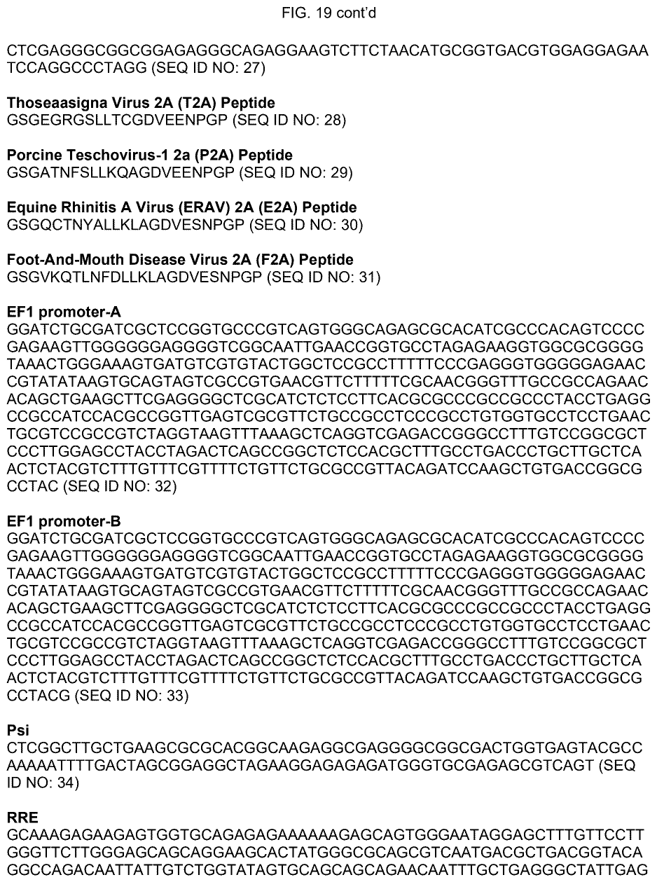

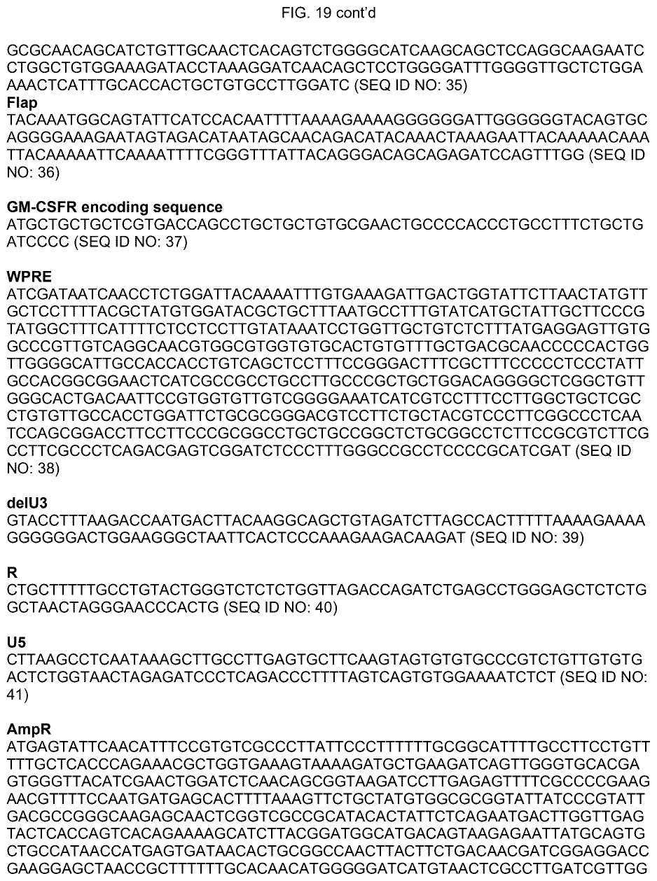

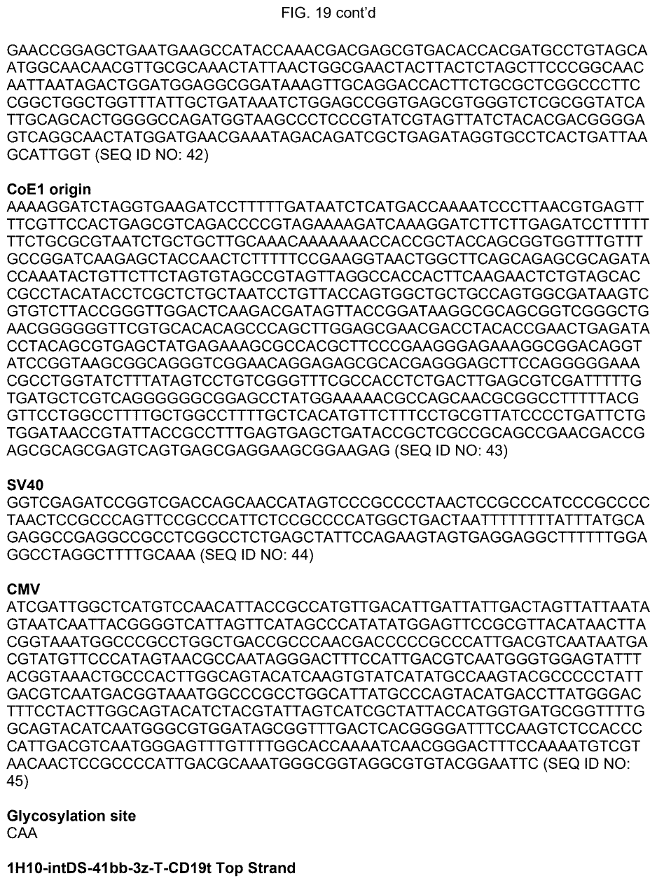

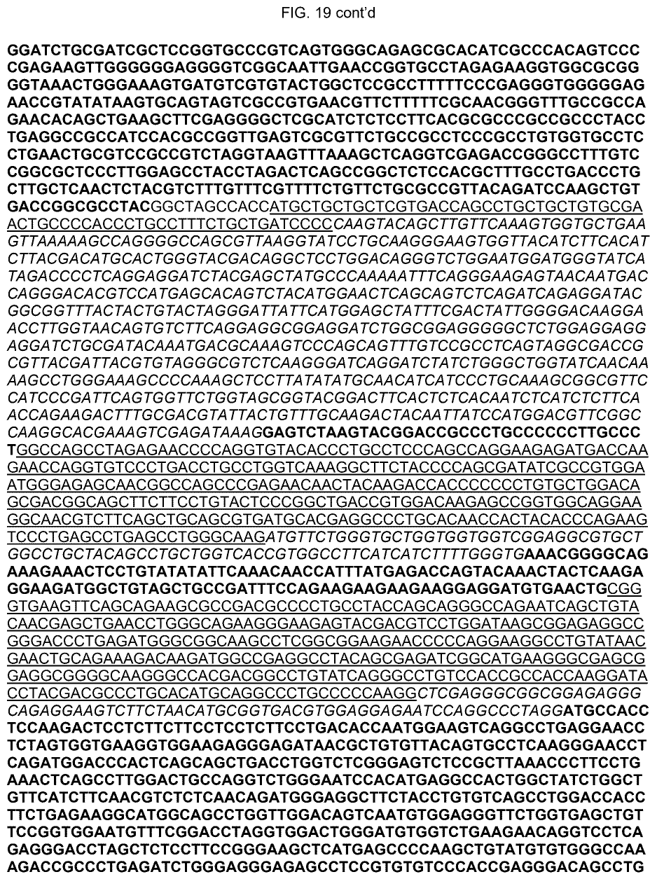

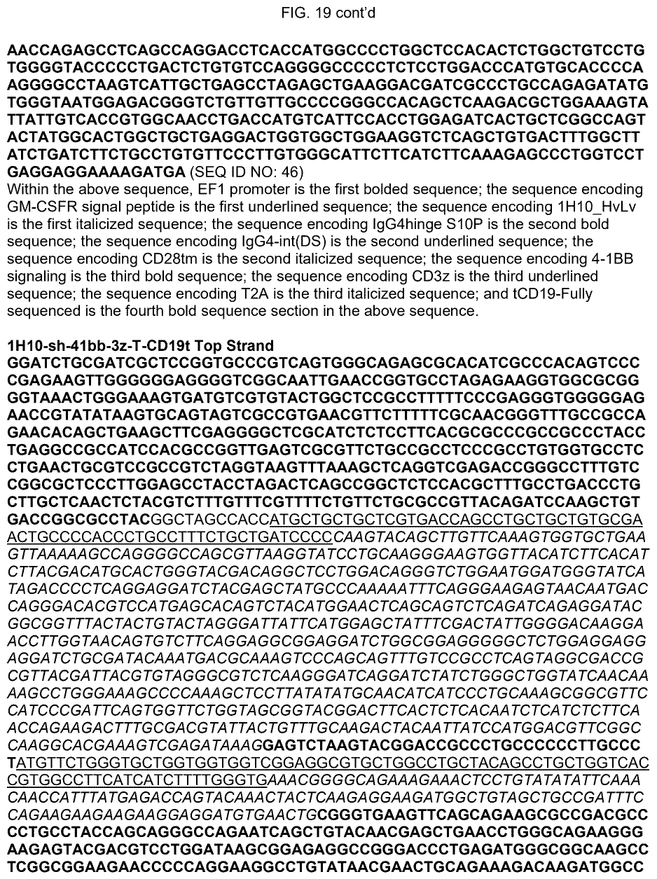

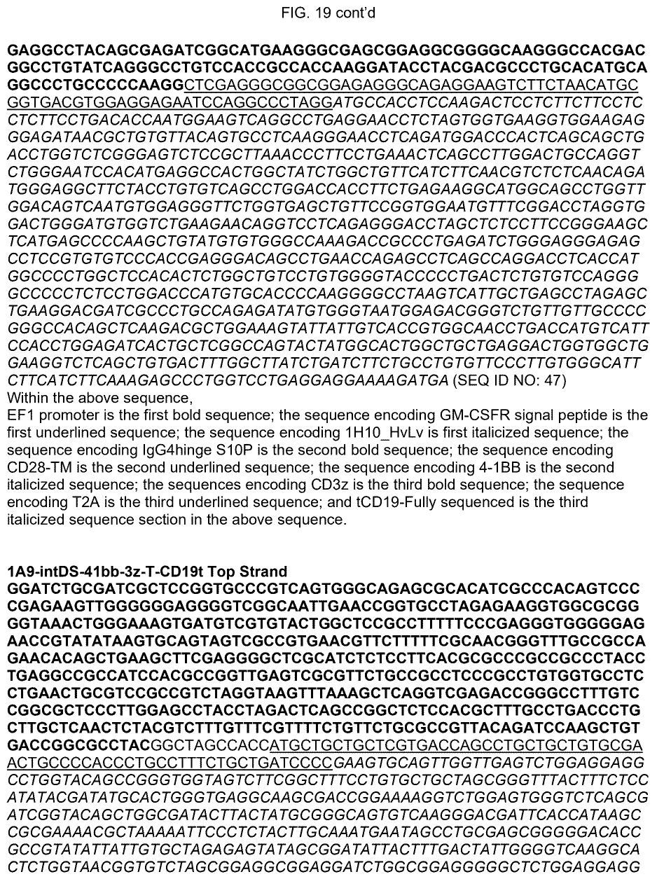

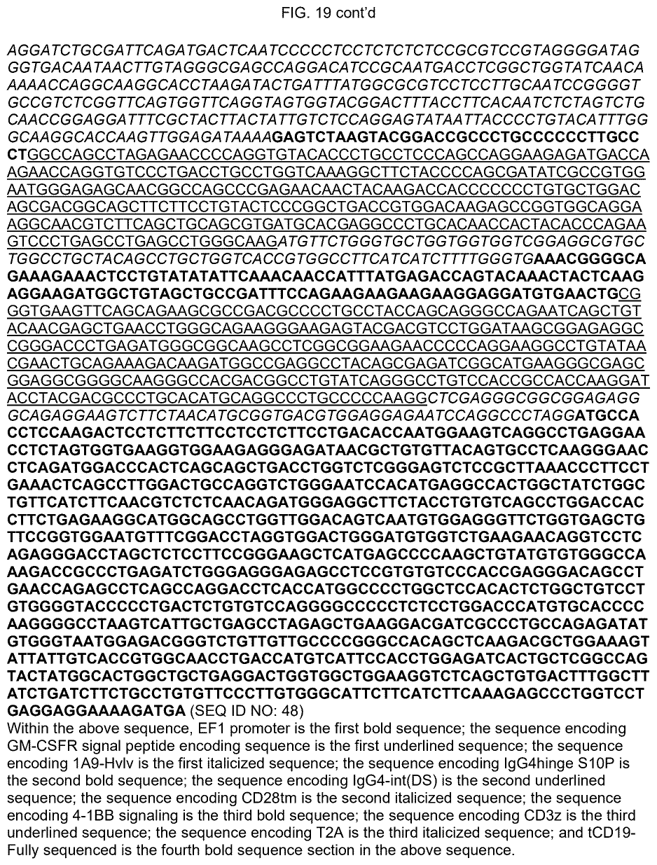

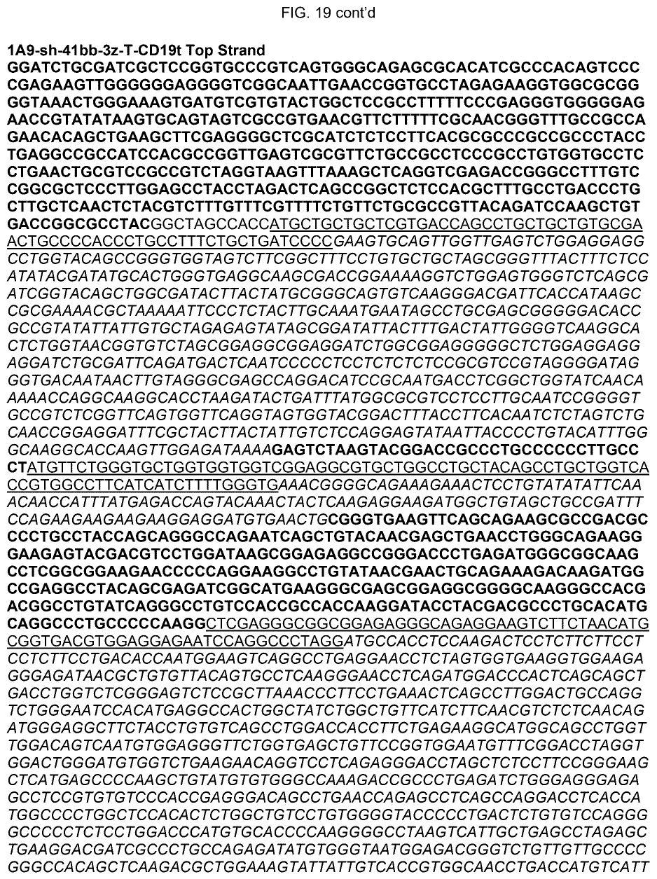

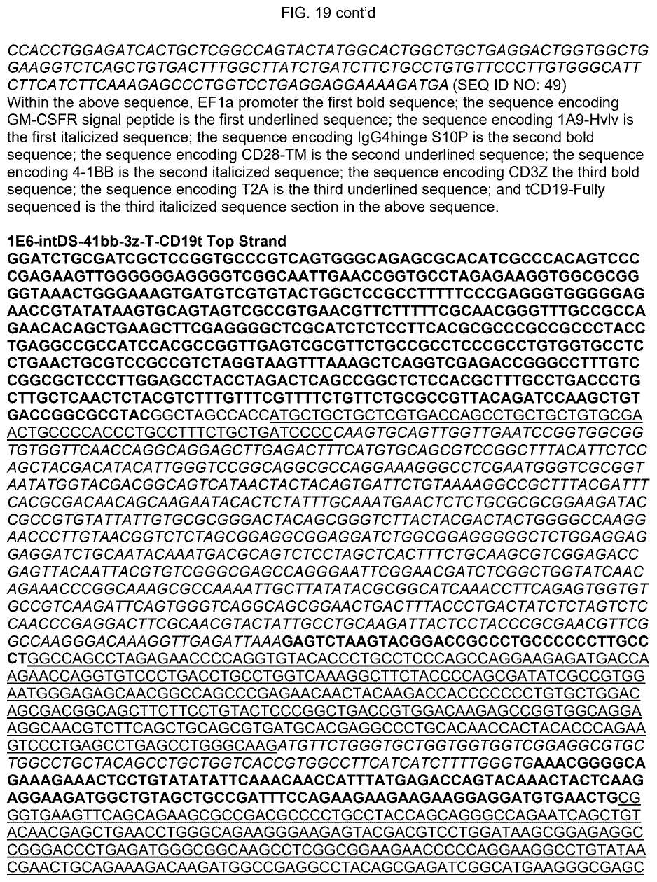

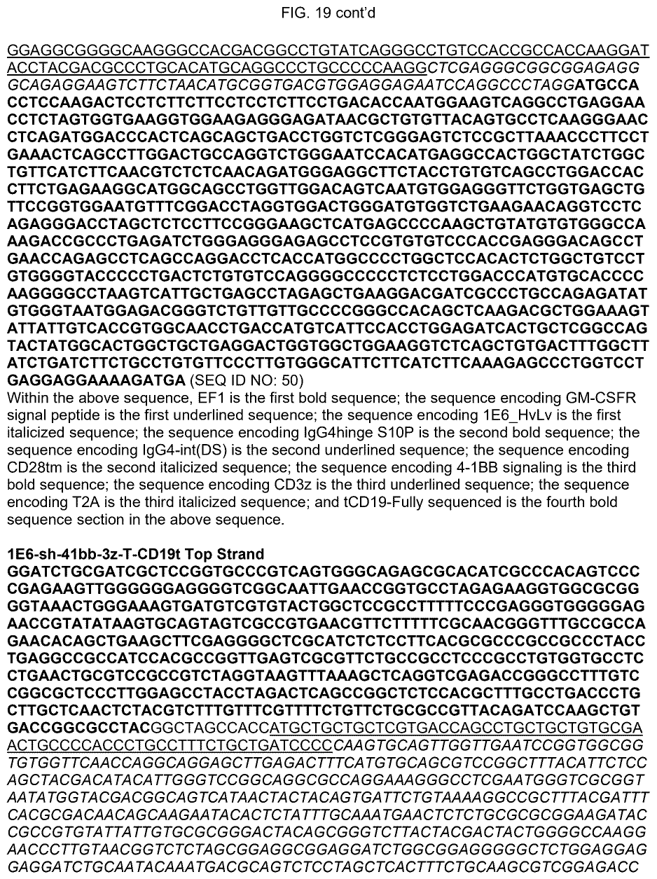

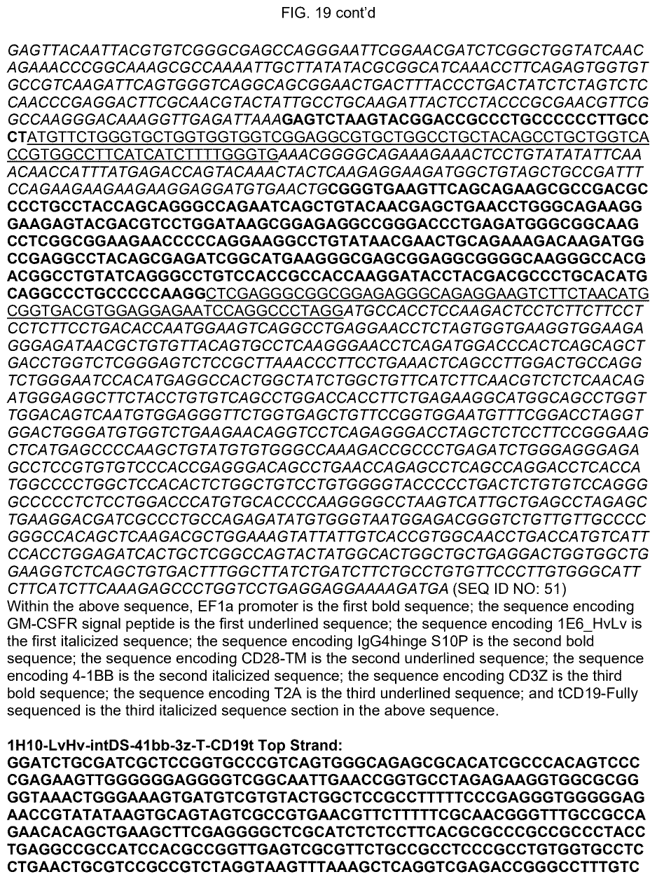

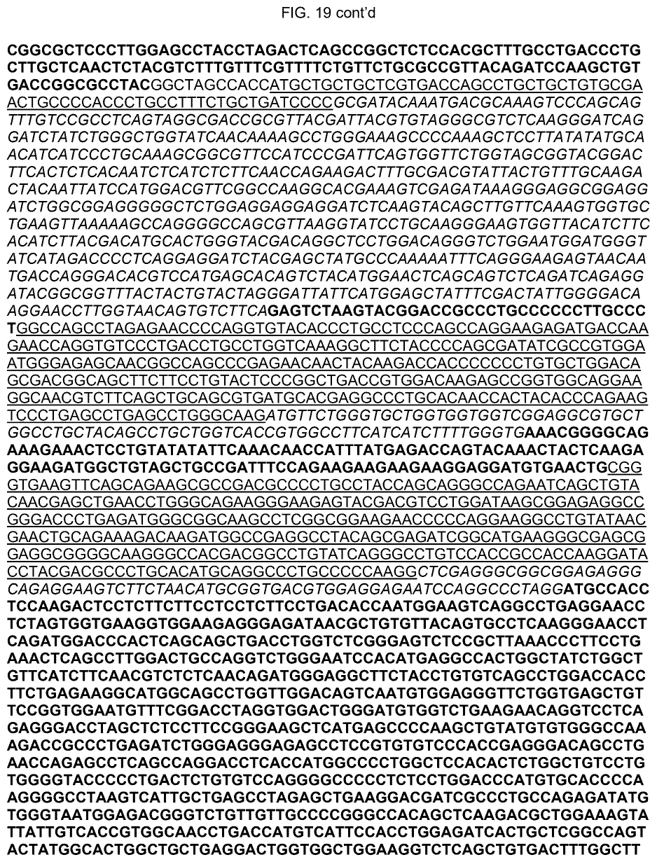

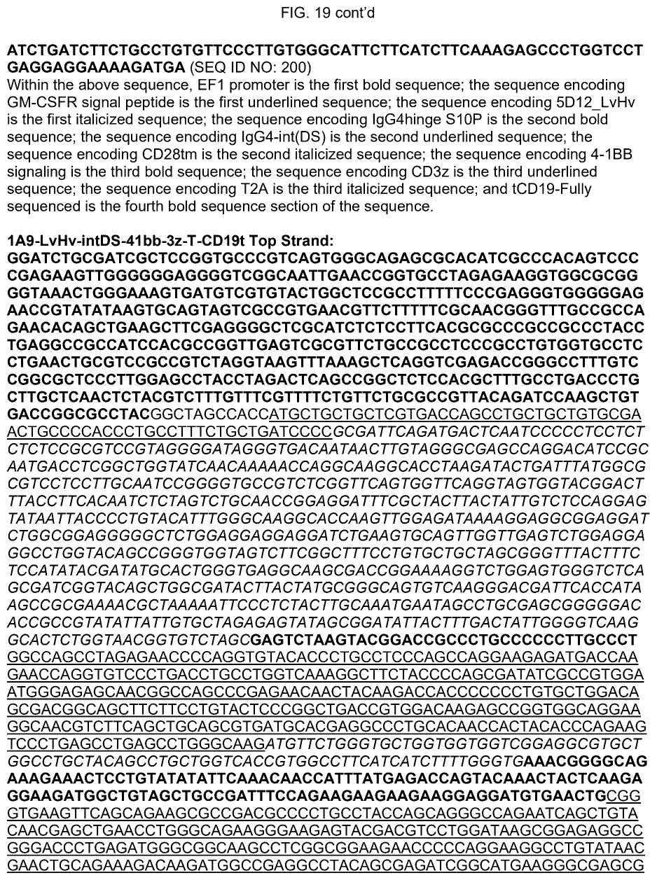

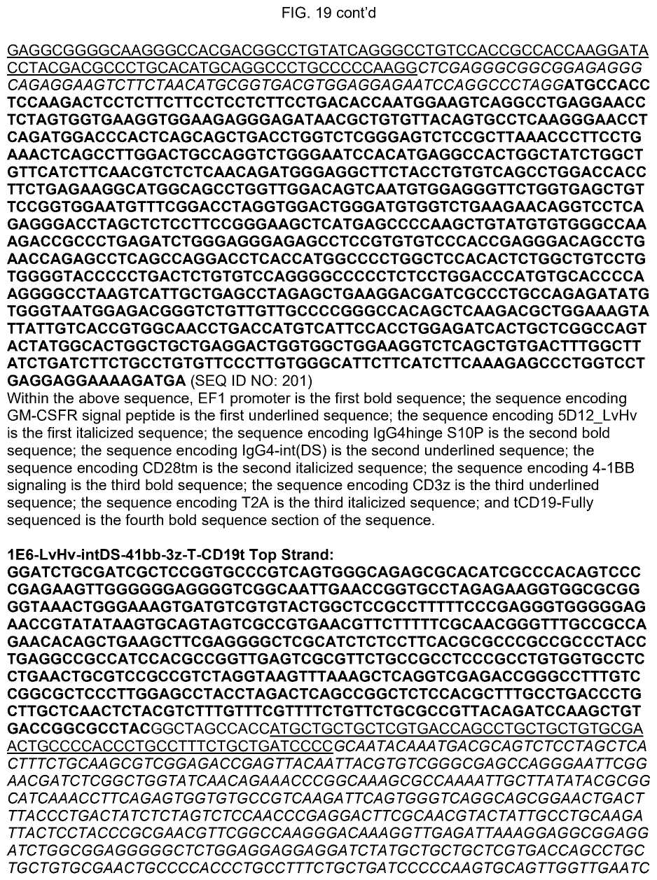

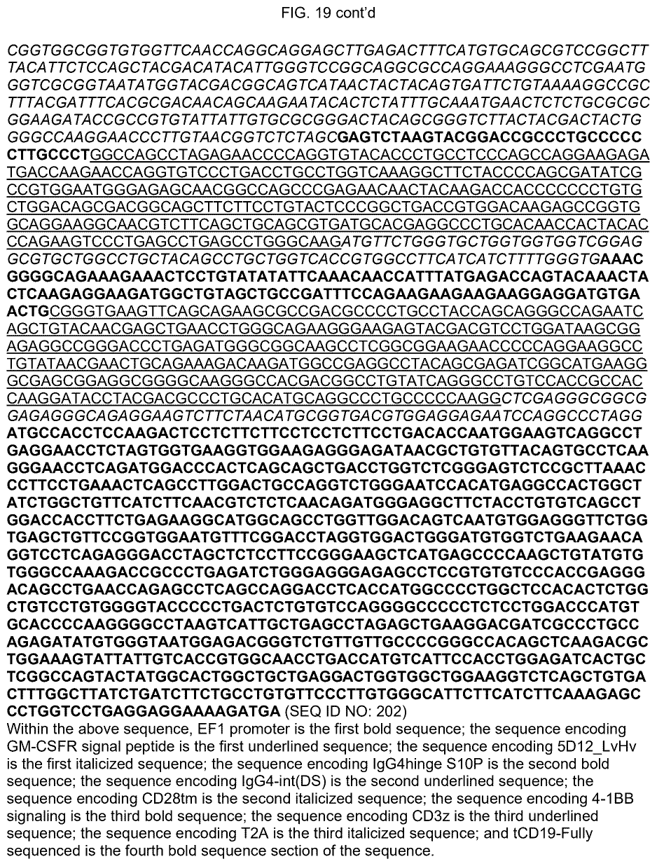

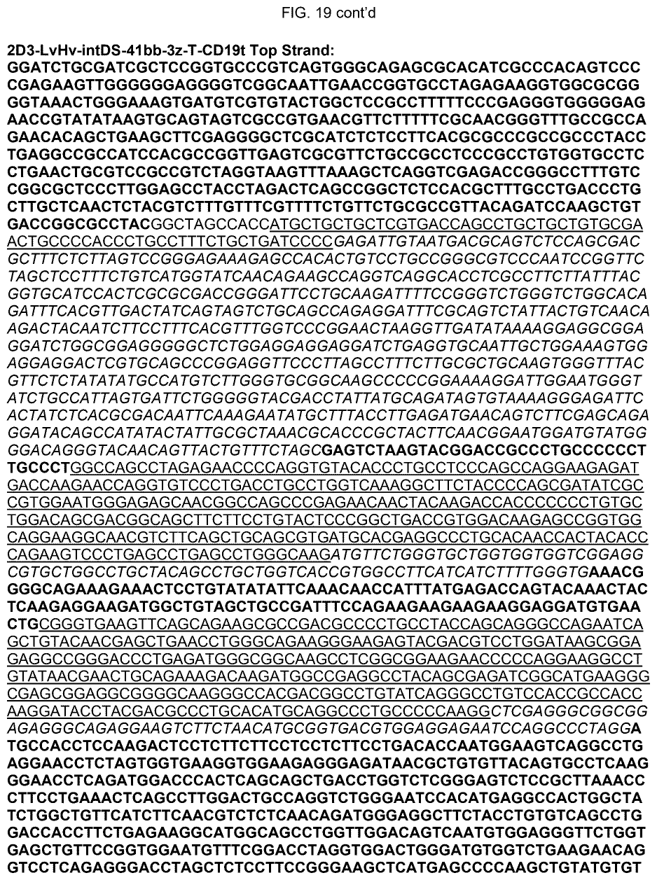

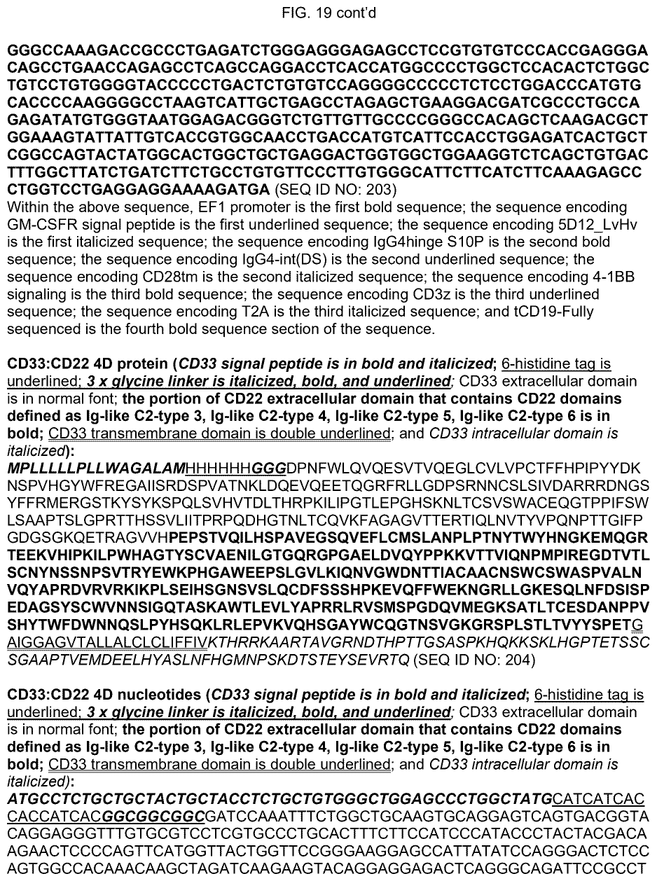

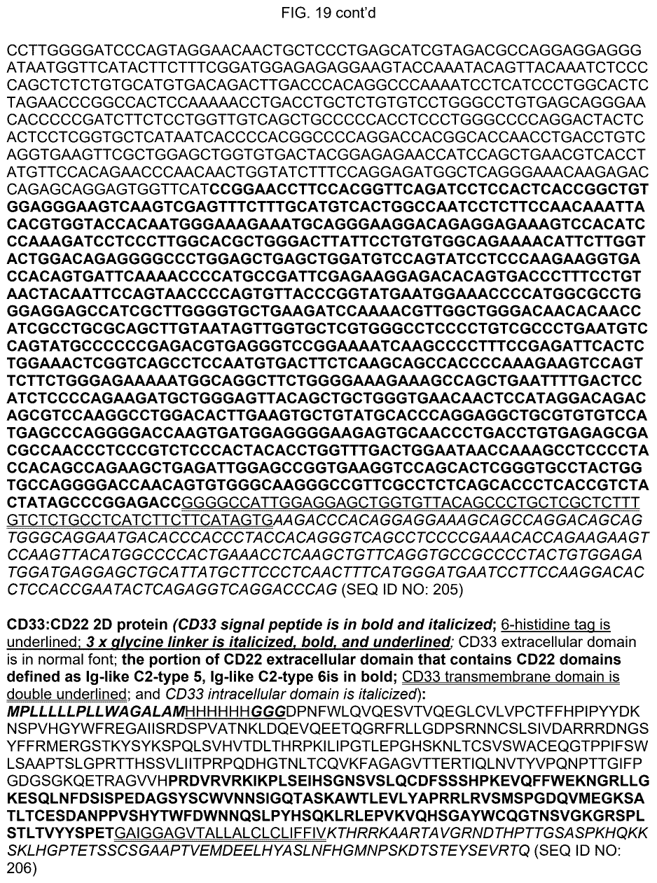

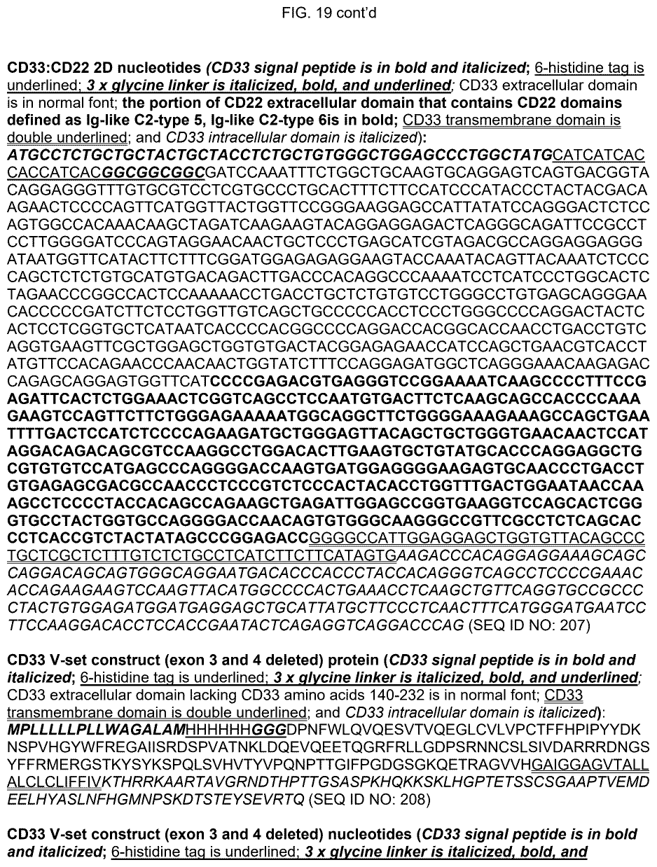

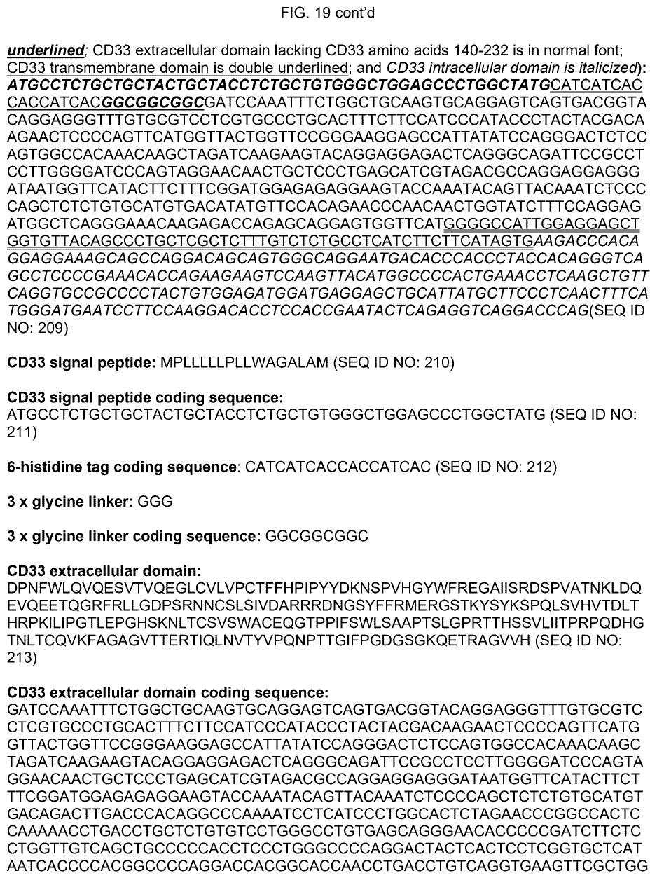

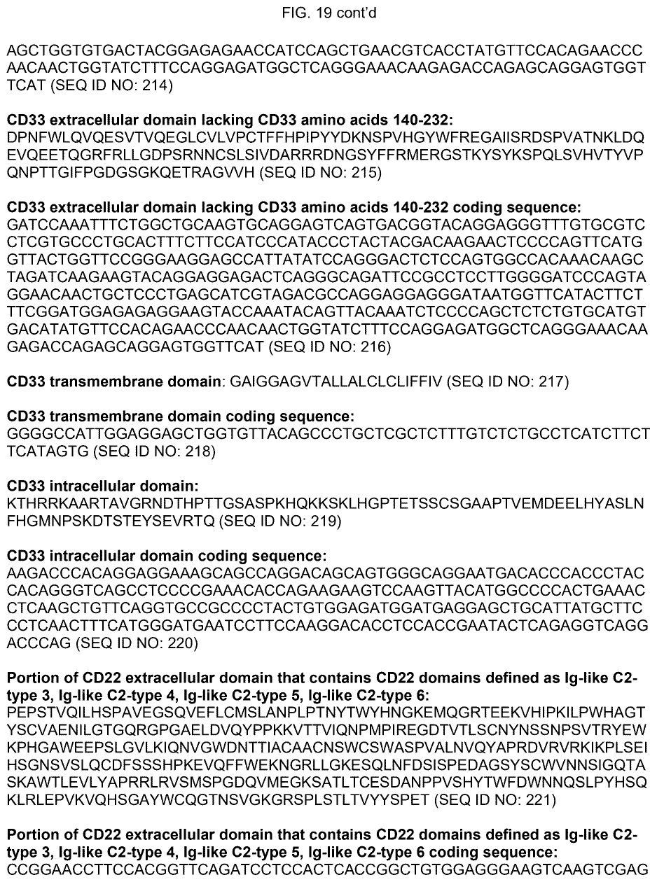

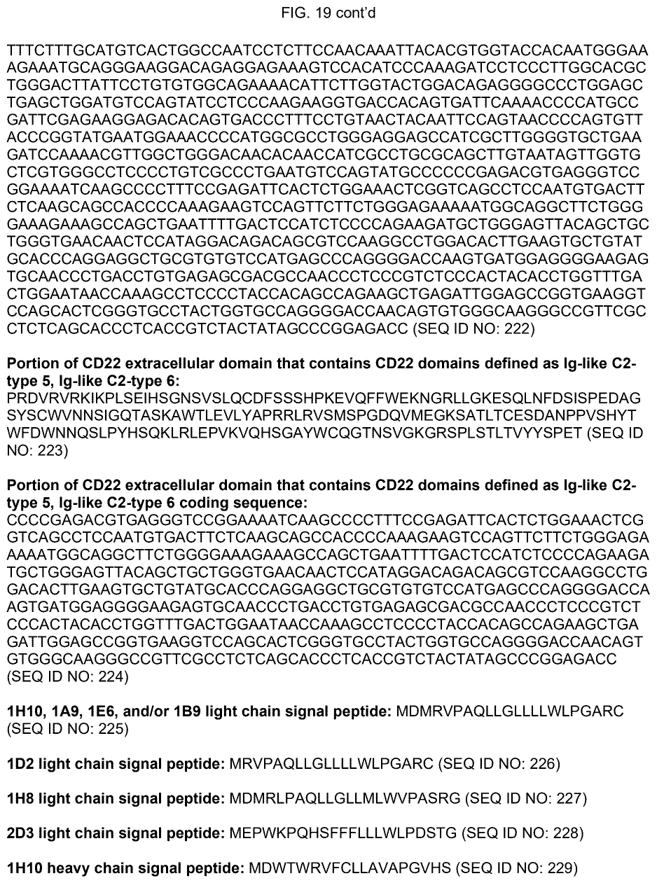

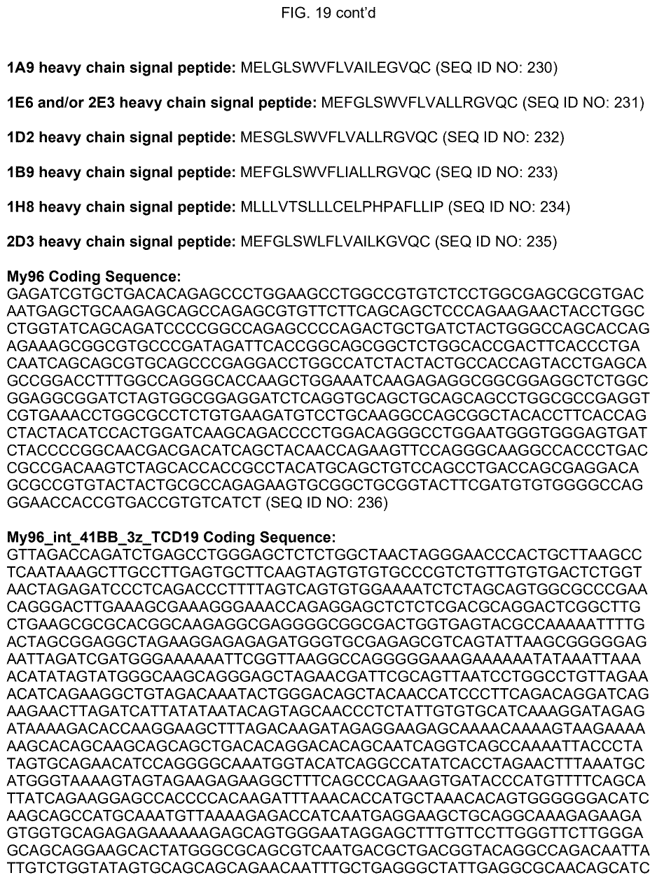

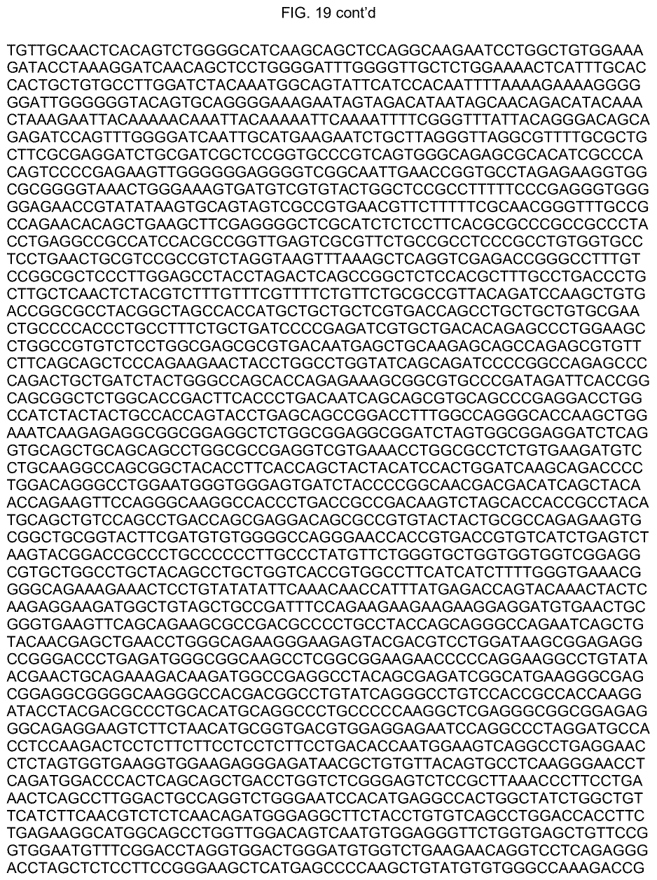

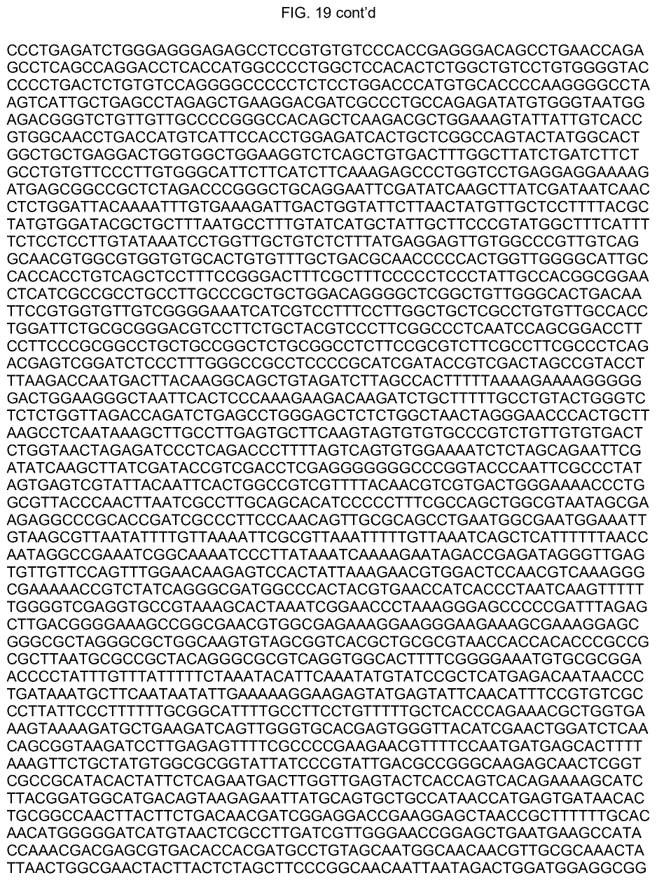

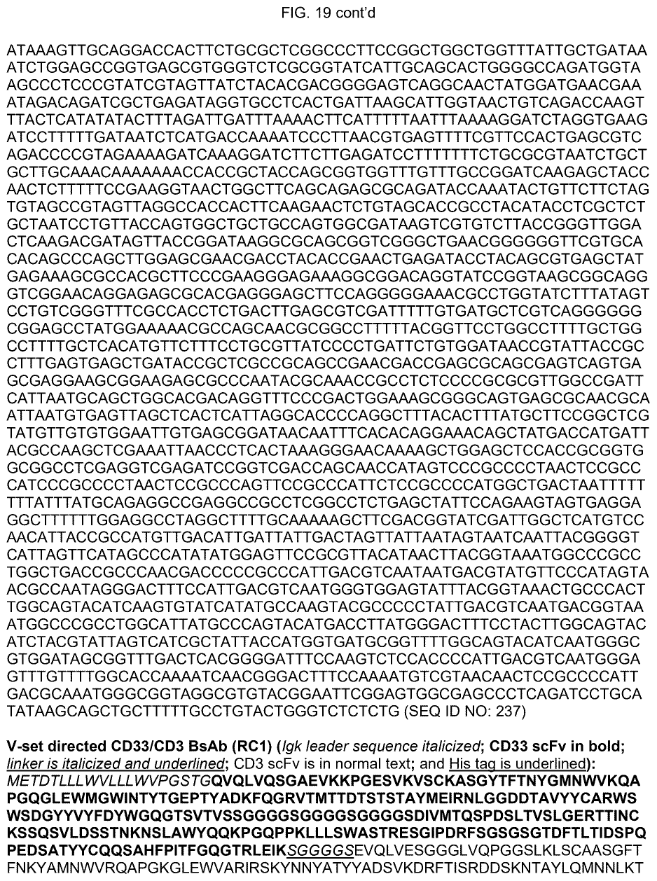

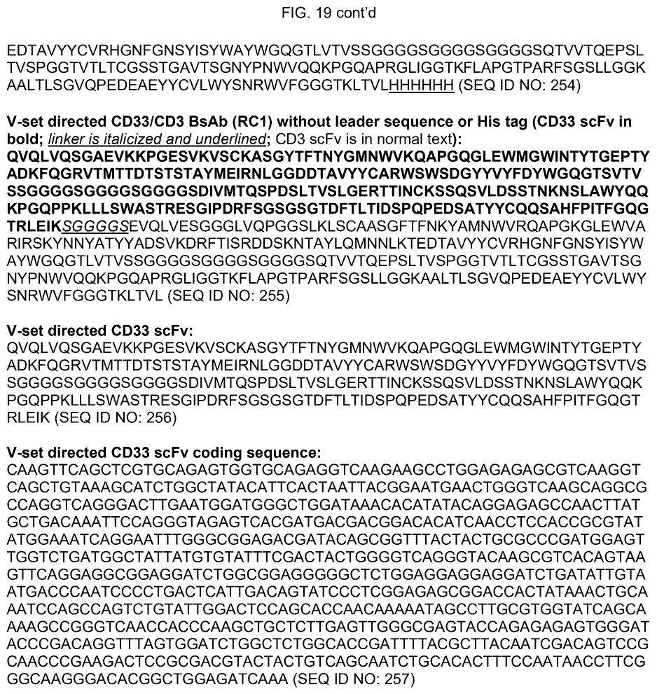

In any of the embodiments described herein, a polynucleotide can include a polynucleotide that encodes a self-cleaving polypeptide, wherein the polynucleotide encoding the self-cleaving polypeptide is located between the polynucleotide encoding the CAR construct and a polynucleotide encoding a transduction marker (e.g., tEGFR). Exemplary self-cleaving polypeptides include 2A peptide from porcine teschovirus-1 (P2A), Thosea asigna virus (T2A), equine rhinitis A virus (E2A), foot-and-mouth disease virus (F2A), or variants thereof (see FIG. 19). Further exemplary nucleic acid and amino acid sequences of 2A peptides are set forth in, for example, Kim et al. (PLOS One 6:e18556 (2011).

A “vector” is a nucleic acid molecule that is capable of transporting another nucleic acid. Vectors may be, e.g., plasmids, cosmids, viruses, or phage. An “expression vector” is a vector that is capable of directing the expression of a protein encoded by one or more genes carried by the vector when it is present in the appropriate environment.

“Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

“Retroviruses” are viruses having an RNA genome. “Gammaretrovirus” refers to a genus of the retroviridae family. Exemplary gammaretroviruses include mouse stem cell virus, murine leukemia virus, feline leukemia virus, feline sarcoma virus, and avian reticuloendotheliosis viruses.

Retroviral vectors (see Miller, et al., 1993, Meth. Enzymol. 217:581-599) can be used. In such embodiments, the gene to be expressed is cloned into the retroviral vector for its delivery into cells. In particular embodiments, a retroviral vector includes all of the cis-acting sequences necessary for the packaging and integration of the viral genome, i.e., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail about retroviral vectors can be found in Boesen, et al., 1994, Biotherapy 6:291-302; Clowes, et al., 1994, J. Clin. Invest. 93:644-651; Kiem, et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114. Adenoviruses, adeno-associated viruses (AAV) and alphaviruses can also be used. See Kozarsky and Wilson, 1993, Current Opinion in Genetics and Development 3:499-503, Rosenfeld, et al., 1991, Science 252:431-434; Rosenfeld, et al., 1992, Cell 68:143-155; Mastrangeli, et al., 1993, J. Clin. Invest. 91:225-234; Walsh, et al., 1993, Proc. Soc. Exp. Bioi. Med. 204:289-300; and Lundstrom, 1999, J. Recept. Signal Transduct. Res. 19: 673-686. Other methods of gene delivery include use of mammalian artificial chromosomes (Vos, 1998, Curr. Op. Genet. Dev. 8:351-359); liposomes (Tarahovsky and Ivanitsky, 1998, Biochemistry (Mosc) 63:607-618); ribozymes (Branch and Klotman, 1998, Exp. Nephrol. 6:78-83); and triplex DNA (Chan and Glazer, 1997, J. Mol. Med. 75:267-282).

There are a large number of available viral vectors suitable within the current disclosure, including those identified for human gene therapy applications (see Pfeifer and Verma, 2001, Ann. Rev. Genomics Hum. Genet. 2:177). Methods of using retroviral and lentiviral viral vectors and packaging cells for transducing mammalian host cells with viral particles including CAR transgenes are described in, e.g., U.S. Pat. No. 8,119,772; Walchli, et al., 2011, PLoS One 6:327930; Zhao, et al., 2005, J. Immunol. 174:4415; Engels, et al., 2003, Hum. Gene Ther. 14:1155; Frecha, et al., 2010, Mol. Ther. 18:1748; and Verhoeyen, et al., 2009, Methods Mol. Biol. 506:97. Retroviral and lentiviral vector constructs and expression systems are also commercially available.

Targeted genetic engineering approaches may also be utilized. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. ZFNs are a class of site-specific nucleases engineered to bind and cleave DNA at specific positions. ZFNs are used to introduce double stranded breaks (DSBs) at a specific site in a DNA sequence which enables the ZFNs to target unique sequences within a genome in a variety of different cells. For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing double DSBs in the DNA, which induce repair mechanisms in cells. Generally, two TALENs must bind and flank each side of the target DNA site for the DNA cleavage domain to dimerize and induce a DSB. For additional information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14(1):49-55; Beurdeley et al., Nat Commun, 2013, 4: 1762; Scharenberg et al., Curr Gene Ther, 2013, 13(4):291-303; Gaj et al., Nat Methods, 2012, 9(8):805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

Particular embodiments can utilize MegaTALs as gene editing agents. MegaTALs have a sc rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Nanoparticles that result in selective in vivo genetic modification of targeted cell types have been described and can be used within the teachings of the current disclosure. In particular embodiments, the nanoparticles can be those described in WO2014153114, WO2017181110, and WO201822672.

(iii-b) CAR Subcomponents. As described previously, CAR molecules include several distinct subcomponents that allow genetically modified cells to recognize and kill unwanted cells, such as cancer cells. The subcomponents include at least an extracellular component and an intracellular component. The extracellular component includes a binding domain that specifically binds a marker that is preferentially present on the surface of unwanted cells. When the binding domain binds such markers, the intracellular component activates the cell to destroy the bound cell. CAR additionally include a transmembrane domain that links the extracellular component to the intracellular component, and other subcomponents that can increase the CAR's function. For example, the inclusion of a spacer region and/or one or more linker sequences can allow the CAR to have additional conformational flexibility, often increasing the binding domain's ability to bind the targeted cell marker.

(iii-b-i) Binding Domains. The current disclosure provides newly developed binding domains for use in CAR based on antibodies that bind CD33. Antibodies are produced from two genes, a heavy chain gene and a light chain gene. Generally, an antibody includes two identical copies of a heavy chain, and two identical copies of a light chain. Within a variable heavy chain and variable light chain, segments referred to as complementary determining regions (CDRs) dictate epitope binding. Each heavy chain has three CDRs (i.e., CDRH1, CDRH2, and CDRH3) and each light chain has three CDRs (i.e., CDRL1, CDRL2, and CDRL3). CDR regions are flanked by framework residues (FR). The precise amino acid sequence boundaries of a given CDR or FR can be readily determined using any of a number of well-known schemes, including those described by: Kabat et al. (1991) “Sequences of Proteins of Immunological Interest,” 5th Ed. Public Health Service, National Institutes of Health, Bethesda, Md. (Kabat numbering scheme); Al-Lazikani et al. (1997) J Mol Biol 273: 927-948 (Chothia numbering scheme); North et al. (2011) J Mol Biol 406(2):228-256 (North numbering scheme); Maccallum et al. (1996) J Mol Biol 262: 732-745 (Contact numbering scheme); Martin et al. (1989) Proc. Natl. Acad. Sci., 86: 9268-9272 (AbM numbering scheme); Lefranc M P et al. (2003) Dev Comp Immunol 27(1): 55-77 (IMGT numbering scheme); and Honegger and Pluckthun (2001) J Mol Biol 309(3): 657-670 (“Aho” numbering scheme). The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. In particular embodiments, the antibody CDR sequences disclosed herein are according to Kabat numbering. CDR residues can be identified using software programs such as ABodyBuilder.

CD33 binding domains for use in CARs are derived from antibodies the 1H10, 1A9, 1E6, 1D2, 1B9, 1 H8, 2D3, or 2E3. In particular embodiments, the antibodies include the following CDR sets. A CDR set refers to 3 light chain CDRs and 3 heavy chain CDRs that together result in binding to CD33.

TABLE 1
Antibody CDR Sequences using North.

| | | | SEQ |
|---|---|---|---|
| Antibody | CDR | SEQUENCE | ID NO: |
| 1H10 | CDRL1 | RASQGIRIYLG | 54 |
| | CDRL2 | YATSSLQS | 55 |
| | CDRL3 | LQDYNYPWT | 56 |
| | CDRH1 | KGSGYIFTSYDMH | 57 |
| | CDRH2 | IIDPSGGSTS | 58 |
| | CDRH3 | TRDYSWSYFDY | 59 |
| 1A9 | CDRL1 | RASQDIRNDLG | 60 |
| | CDRL2 | YGASSLQS | 61 |
| | CDRL3 | LQEYNYPCT | 62 |
| | CDRH1 | AASGFTFSIYDMH | 63 |
| | CDRH2 | AIGTAGDTY | 64 |
| | CDRH3 | AREYSGYYFDY | 65 |
| 1E6 | CDRL1 | RASQGIRNDLG | 66 |
| | CDRL2 | YAASNLQS | 67 |
| | CDRL3 | LQDYSYPRT | 68 |
| | CDRH1 | AASGFTFSSYDIH | 69 |
| | CDRH2 | VIWYDGSHNY | 70 |
| | CDRH3 | ARDYSGSYYDY | 71 |
| 1D2 | CDRL1 | RASQGIRNDLG | 66 |
| | CDRL2 | YATSSLQS | 55 |
| | CDRL3 | LQDYSYPRT | 68 |
| | CDRH1 | AASGFTFSSYDIH | 69 |
| | CDRH2 | VIWYDGSQKY | 72 |
| | CDRH3 | ARDYSGSYYDY | 71 |
| 1B9 | CDRL1 | RASQDIRNDLG | 60 |
| | CDRL2 | YAASSLQS | 73 |
| | CDRL3 | LQDYSYPRT | 68 |
| | CDRH1 | AASGFIFSSYDIH | 74 |
| | CDRH2 | VIWYDGSHNY | 70 |
| | CDRH3 | ARDYSGSYFDY | 75 |
| 1H8 | CDRL1 | RASQNIGGNLH | 76 |
| | CDRL2 | RYATQPFS | 93 |
| | CDRL3 | HQSSSLPLT | 78 |
| | CDRH1 | AASGFTFGSYGMH | 94 |
| | CDRH2 | VIWYDGSNEY | 95 |
| | CDRH3 | ARDLDYDSSGGDY | 96 |
| 2D3 | CDRL1 | RASQSGSSSFLS | 97 |
| | CDRL2 | YGASTRAT | 98 |
| | CDRL3 | QQDYNLPFT | 84 |
| | CDRH1 | AASGFTFSIYAMS | 99 |
| | CDRH2 | AISDSGGTTY | 100 |
| | CDRH3 | AKRTRYFNGMDV | 101 |
| 2E3 | CDRL1 | RASQSVSSSYLA | 102 |
| | CDRL2 | YGTSSRAT | 103 |
| | CDRL3 | QQYGSSPT | 90 |
| | CDRH1 | AASGFTFSSYGMH | 104 |
| | CDRH2 | VIWYGGSNKY | 105 |
| | CDRH3 | ARDGTGENYYYYVMDV | 106 |

TABLE 2
Antibody CDR Sequences using IMGT

| | | | SEQ |
|---|---|---|---|
| Antibody | CDR | SEQUENCE | ID NO: |
| 1H10 | CDRL1 | QGIRIY | 107 |
| | CDRL2 | ATS | N/A |
| | CDRL3 | LQDYNYPWT | 56 |
| | CDRH1 | GYIFTSYD | 108 |
| | CDRH2 | IDPSGGST | 109 |
| | CDRH3 | TRDYSWSYFDY | 59 |
| 1A9 | CDRL1 | QDIRND | 110 |
| | CDRL2 | GAS | N/A |
| | CDRL3 | LQEYNYPCT | 62 |
| | CDRH1 | GFTFSIYD | 111 |
| | CDRH2 | IGTAGDT | 112 |
| | CDRH3 | AREYSGYYFDY | 65 |
| 1E6 | CDRL1 | QGIRND | 113 |
| | CDRL2 | AAS | N/A |
| | CDRL3 | LQDYSYPRT | 68 |
| | CDRH1 | GFTFSSYD | 114 |
| | CDRH2 | IWYDGSHN | 115 |
| | CDRH3 | ARDYSGSYYDY | 71 |
| 1D2 | CDRL1 | QGIRND | 113 |
| | CDRL2 | ATS | N/A |
| | CDRL3 | LQDYSYPRT | 68 |
| | CDRH1 | GFTFSSYD | 114 |
| | CDRH2 | IWYDGSQK | 116 |
| | CDRH3 | ARDYSGSYYDY | 71 |
| 1B9 | CDRL1 | QDIRND | 110 |
| | CDRL2 | AAS | N/A |
| | CDRL3 | LQDYSYPRT | 68 |
| | CDRH1 | GFIFSSYD | 117 |
| | CDRH2 | IWYDGSHN | 115 |
| | CDRH3 | ARDYSGSYFDY | 75 |
| 1H8 | CDRL1 | QNIGGN | 118 |
| | CDRL2 | YAT | N/A |
| | CDRL3 | HQSSSLPLT | 78 |
| | CDRH1 | GFTFGSYG | 119 |
| | CDRH2 | IWYDGSNE | 120 |
| | CDRH3 | ARDLDYDSSGGDY | 96 |
| 2D3 | CDRL1 | QSGSSSF | 121 |
| | CDRL2 | GAS | N/A |
| | CDRL3 | QQDYNLPFT | 84 |
| | CDRH1 | GFTFSIYA | 122 |
| | CDRH2 | ISDSGGTT | 123 |
| | CDRH3 | AKRTRYFNGMDV | 101 |
| 2E3 | CDRL1 | QSVSSSY | 124 |
| | CDRL2 | GTS | N/A |
| | CDRL3 | QQYGSSPT | 90 |
| | CDRH1 | GFTFSSYG | 125 |
| | CDRH2 | IWYGGSNK | 126 |
| | CDRH3 | ARDGTGENYYYYVMDV | 106 |

TABLE 3
Antibody CDR Sequences using Kabat.

| | | | SEQ |
|---|---|---|---|
| Antibody | CDR | SEQUENCE | ID NO: |
| 1H10 | CDRL1 | RASQGIRIYLG | 54 |
| | CDRL2 | ATSSLQS | 127 |
| | CDRL3 | LQDYNYPWT | 56 |
| | CDRH1 | SYDMH | 128 |
| | CDRH2 | IIDPSGGSTSYAQKFQG | 129 |
| | CDRH3 | DYSWSYFDY | 130 |
| 1A9 | CDRL1 | RASQDIRNDLG | 60 |
| | CDRL2 | GASSLQS | 131 |
| | CDRL3 | LQEYNYPCT | 62 |
| | CDRH1 | IYDMH | 132 |
| | CDRH2 | AIGTAGDTYYAGSVKG | 133 |
| | CDRH3 | EYSGYYFDY | 134 |
| 1E6 | CDRL1 | RASQGIRNDLG | 66 |
| | CDRL2 | AASNLQS | 135 |
| | CDRL3 | LQDYSYPRT | 68 |
| | CDRH1 | SYDIH | 136 |
| | CDRH2 | VIWYDGSHNYYSDSVKG | 137 |
| | CDRH3 | DYSGSYYDY | 138 |
| 1D2 | CDRL1 | RASQGIRNDLG | 66 |
| | CDRL2 | ATSSLQS | 127 |
| | CDRL3 | LQDYSYPRT | 68 |
| | CDRH1 | SYDIH | 136 |
| | CDRH2 | VIWYDGSQKYYADSVKG | 139 |
| | CDRH3 | DYSGSYYDY | 138 |
| 1B9 | CDRL1 | RASQDIRNDLG | 60 |
| | CDRL2 | AASSLQS | 140 |
| | CDRL3 | LQDYSYPRT | 68 |
| | CDRH1 | SYDIH | 136 |
| | CDRH2 | VIWYDGSHNYYSDSVKG | 137 |
| | CDRH3 | DYSGSYFDY | 141 |
| 1H8 | CDRL1 | RASQNIGGNLH | 76 |
| | CDRL2 | YATQPFS | 77 |
| | CDRL3 | HQSSSLPLT | 78 |
| | CDRH1 | SYGMH | 79 |
| | CDRH2 | VIWYDGSNEYYADSVKG | 142 |
| | CDRH3 | DLDYDSSGGDY | 143 |
| 2D3 | CDRL1 | RASQSGSSSFLS | 97 |
| | CDRL2 | GASTRAT | 83 |
| | CDRL3 | QQDYNLPFT | 84 |
| | CDRH1 | IYAMS | 85 |
| | CDRH2 | AISDSGGTTYYADSVKG | 144 |
| | CDRH3 | RTRYFNGMDV | 145 |
| 2E3 | CDRL1 | RASQSVSSSYLA | 102 |
| | CDRL2 | GTSSRAT | 89 |
| | CDRL3 | QQYGSSPT | 90 |
| | CDRH1 | SYGMH | 79 |
| | CDRH2 | VIWYGGSNKYYADSVKG | 146 |
| | CDRH3 | DGTGENYYYYVMDV | 147 |

TABLE 4
Antibody CDR Sequences using Chothia

| | | | SEQ |
|---|---|---|---|
| Antibody | CDR | SEQUENCE | ID NO: |
| 1H10 | CDRL1 | RASQGIRIYLG | 54 |
| | CDRL2 | ATSSLQS | 127 |
| | CDRL3 | LQDYNYPWT | 56 |
| | CDRH1 | GYIFTSY | 148 |
| | CDRH2 | DPSGGS | 149 |
| | CDRH3 | DYSWSYFDY | 130 |
| 1A9 | CDRL1 | RASQDIRNDLG | 60 |
| | CDRL2 | GASSLQS | 131 |
| | CDRL3 | LQEYNYPCT | 62 |
| | CDRH1 | GFTFSIY | 150 |
| | CDRH2 | GTAGD | 151 |
| | CDRH3 | EYSGYYFDY | 134 |
| 1E6 | CDRL1 | RASQGIRNDLG | 66 |
| | CDRL2 | AASNLQS | 135 |
| | CDRL3 | LQDYSYPRT | 68 |
| | CDRH1 | GFTFSSY | 152 |
| | CDRH2 | WYDGSH | 153 |
| | CDRH3 | DYSGSYYDY | 138 |
| 1D2 | CDRL1 | RASQGIRNDLG | 66 |
| | CDRL2 | ATSSLQS | 127 |
| | CDRL3 | LQDYSYPRT | 68 |
| | CDRH1 | GFTFSSY | 152 |
| | CDRH2 | WYDGSQ | 154 |
| | CDRH3 | DYSGSYYDY | 138 |
| 1B9 | CDRL1 | RASQDIRNDLG | 60 |
| | CDRL2 | AASSLQS | 140 |
| | CDRL3 | LQDYSYPRT | 68 |
| | CDRH1 | GFIFSSY | 155 |
| | CDRH2 | WYDGSH | 153 |
| | CDRH3 | DYSGSYFDY | 141 |
| 1H8 | CDRL1 | RASQNIGGNLH | 76 |
| | CDRL2 | YATQPFS | 77 |
| | CDRL3 | HQSSSLPLT | 78 |
| | CDRH1 | GFTFGSY | 156 |
| | CDRH2 | WYDGSN | 157 |
| | CDRH3 | DLDYDSSGGDY | 143 |
| 2D3 | CDRL1 | RASQSGSSSFLS | 97 |
| | CDRL2 | GASTRAT | 83 |
| | CDRL3 | QQDYNLPFT | 84 |
| | CDRH1 | GFTFSIY | 150 |
| | CDRH2 | SDSGGT | 158 |
| | CDRH3 | RTRYFNGMDV | 145 |
| 2E3 | CDRL1 | RASQSVSSSYLA | 102 |
| | CDRL2 | GTSSRAT | 89 |
| | CDRL3 | QQYGSSPT | 90 |
| | CDRH1 | GFTFSSY | 152 |
| | CDRH2 | WYGGSN | 159 |
| | CDRH3 | DGTGENYYYYVMDV | 147 |

TABLE 5
Antibody CDR Sequences-Set 5.

| | | | SEQ |
|---|---|---|---|
| Antibody | CDR | SEQUENCE | ID NO: |
| 1H10 | CDRL1 | RASQGIRIYLG | 54 |
| | CDRL2 | YATSSLQS | 55 |
| | CDRL3 | LQDYNYPWT | 56 |
| | CDRH1 | KGSGYIFTSYDMH | 57 |
| | CDRH2 | IIDPSGGSTS | 58 |
| | CDRH3 | TRDYSWSYFDY | 59 |
| 1A9 | CDRL1 | RASQDIRNDLG | 60 |
| | CDRL2 | YGASSLQS | 61 |
| | CDRL3 | LQEYNYPCT | 62 |
| | CDRH1 | AASGFTFSIYDMH | 63 |
| | CDRH2 | AIGTAGDTY | 64 |
| | CDRH3 | AREYSGYYFDY | 65 |
| 1E6 | CDRL1 | RASQGIRNDLG | 66 |
| | CDRL2 | YAASNLQS | 67 |
| | CDRL3 | LQDYSYPRT | 68 |
| | CDRH1 | AASGFTFSSYDIH | 69 |
| | CDRH2 | VIWYDGSHNY | 70 |
| | CDRH3 | ARDYSGSYYDY | 71 |
| 1D2 | CDRL1 | RASQGIRNDLG | 66 |
| | CDRL2 | YATSSLQS | 55 |
| | CDRL3 | LQDYSYPRT | 68 |
| | CDRH1 | AASGFTFSSYDIH | 69 |
| | CDRH2 | VIWYDGSQKY | 72 |
| | CDRH3 | ARDYSGSYYDY | 71 |
| 1B9 | CDRL1 | RASQDIRNDLG | 60 |
| | CDRL2 | YAASSLQS | 73 |
| | CDRL3 | LQDYSYPRT | 68 |
| | CDRH1 | AASGFIFSSYDIH | 74 |
| | CDRH2 | VIWYDGSHNY | 70 |
| | CDRH3 | ARDYSGSYFDY | 75 |
| 1H8 | CDRL1 | RASQNIGGNLH | 76 |
| | CDRL2 | YATQPFS | 77 |
| | CDRL3 | HQSSSLPLT | 78 |
| | CDRH1 | SYGMH | 79 |
| | CDRH2 | IWYDGSNEYYADSVKG | 80 |
| | CDRH3 | DLDYDSSG | 81 |
| 2D3 | CDRL1 | QSGSSSFLS | 82 |
| | CDRL2 | GASTRAT | 83 |
| | CDRL3 | QQDYNLPFT | 84 |
| | CDRH1 | IYAMS | 85 |
| | CDRH2 | ISDSGGTTYYADSVKG | 86 |
| | CDRH3 | RTRYFNG | 87 |
| 2E3 | CDRL1 | QSVSSSYLA | 88 |
| | CDRL2 | GTSSRAT | 89 |
| | CDRL3 | QQYGSSPT | 90 |
| | CDRH1 | SYGMH | 79 |
| | CDRH2 | IWYGGSNKYYADSVKG | 91 |
| | CDRH3 | DGTGENYYYYV | 92 |

Particular embodiments include an scFV derived from the CDRs, VL or VH of 1H10, 1A9; 1E6; 102; 11B9; 11 H8; 203; or 2E3 for use in a CAR. Examples of such scFvs are provided in FIG. 19. scFvs can be formed in a VH-VL orientation or a VL-VH orientation. scFvs for use in CAR can also be formulated from the variable chains of these antibodies.

In particular embodiments, the 1H10 antibody includes a variable light chain including the sequence: AIQMTQSPSSLSASVGDRVTITCRASQGIRIYLGWYQQKPGKAPKLLIYATSSLQSGVPSRFSG SGSGTDFTLTISSLQPEDFATYYCLQDYNYPWTFGQGTKVEIK (SEQ ID NO: 238) and a variable heavy chain including the sequence:

(SEQ ID NO: 239)
QVQLVQSGAEVKKPGASVKVSCKGSGYIFTSYDMHWVRQAPGQGLEW
MGIIDPSGGSTSYAQKFQGRVTMTRDTSMSTVYMELSSLRSEDTAVY
YCTRDYSWSYFDYWGQGTLVTVSS.

In particular embodiments, the 1A9 antibody includes a variable light chain including the sequence: AIQMTQSPSSLSASVGDRVTITCRASQDIRNDLGWYQQKPGKAPKILIYGASSLQSGVPSRFSG SGSGTDFTFTISSLQPEDFATYYCLQEYNYPCTFGQGTKLEIK (SEQ ID NO: 240) and a variable heavy chain including the sequence:

(SEQ ID NO: 241)
EVQLVESGGGLVQPGGSLRLSCAASGFTFSIYDMHWVRQATGKGLEW
VSAIGTAGDTYYAGSVKGRFTISRENAKNSLYLQMNSLRAGDTAVYY
CAREYSGYYFDYWGQGTLVTVSS.

In particular embodiments, the 1E6 antibody includes a variable light chain including the sequence: AIQMTQSPSSLSASVGDRVTITCRASQGIRNDLGWYQQKPGKAPKLLIYAASNLQSGVPSRFS GSGSGTDFTLTISSLQPEDFATYYCLQDYSYPRTFGQGTKVEIK (SEQ ID NO: 242) and a variable heavy chain including the sequence:

(SEQ ID NO: 243)
QVQLVESGGGVVQPGRSLRLSCAASGFTFSSYDIHWVRQAPGKGLEW
VAVIWYDGSHNYYSDSVKGRFTISRDNSKNTLYLQMNSLRAEDTAVY
YCARDYSGSYYDYWGQGTLVTVSS.

In particular embodiments, the 1D2 antibody includes a variable light chain including the sequence: AIQMTQSPSSLSASVGDRVTITCRASQGIRNDLGWYQQKPGKAPELLIYATSSLQSGVPSRFSG SGSGTDFTLIISSLQPEDFATYYCLQDYSYPRTFGQGTKVEIK (SEQ ID NO: 244) and a variable heavy chain including the sequence:

(SEQ ID NO: 245)
QVQLVESGGGVVQPGRSLRLSCAASGFTFSSYDIHWVRQAPGKGLEW
VAVIWYDGSQKYYADSVKGRFTISRDNSKNTLYLQMNSLRAEDTAVY
YCARDYSGSYYDYWGQGTLVTVSS.

In particular embodiments, the 1B9 antibody includes a variable light chain including the sequence: AIQMTQSPSSLSASVGDRVTITCRASQDIRNDLGWYLQRPGKAPKLLIYAASSLQSGVPSRFSG SGSGTDFTLTISSLQPEDFATYYCLQDYSYPRTFGQGTTVEIK (SEQ ID NO: 246) and a variable heavy chain including the sequence:

(SEQ ID NO: 247)
QVQLVESGGGVVQPGRSLRLSCAASGFIFSSYDIHWVRQAPGKGLEW
VAVIWYDGSHNYYSDSVKGRFTISRDNSKNTLYLQMNSLRAEDTAVY
YCARDYSGSYFDYWGQGTLVTVSS.

In particular embodiments, the CD33^{V-set} antibody includes 1H8. In particular embodiments, the 1H8 antibody includes a variable light chain including the sequence: EIVLTQSPDFQSVTPKEKVTITCRASQNIGGNLHWYQQKPDQSPKLLIRYATQPFSGVPSRFGG SGSGTDFTLTINSLEAEDAATYYCHQSSSLPLTFGGGTKVEIK (SEQ ID NO: 248) and a variable heavy chain including the sequence:

(SEQ ID NO: 249)
QVQLVESGGGVVQPGGSLRLSCAASGFTFGSYGMHWVRQAPGKGLEW
VAVIWYDGSNEYYADSVKGRFTVSRDNSKHTLYLQMNRLRAEDTAVY
YCARDLDYDSSGGDYWGQGILVLVSS.

In particular embodiments, the CD33^{V-set} antibody includes 2D3. In particular embodiments, the 2D3 antibody includes a variable light chain including the sequence: EIVMTQSPATLSLSPGERATLSCRASQSGSSSFLSWYQQKPGQAPRLLIYGASTRATGIPARFS GSGSGTDFTLTISSLQPEDFAVYYCQQDYNLPFTFGPGTKVDIK (SEQ ID NO: 250) and a variable heavy chain including the sequence:

(SEQ ID NO: 251)
EVQLLESGGGLVQPGGSLSLSCAASGFTFSIYAMSWVRQAPGKGLEW
VSAISDSGGTTYYADSVKGRFTISRDNSKNMLYLEMNSLRAEDTAIY
YCAKRTRYFNGMDVWGQGTTVTVSS.

In particular embodiments, the CD33-set antibody includes 2E3. In particular embodiments, the 2E3 antibody includes a variable light chain including the sequence: EIVLTQSPGTLSLSPGERATLSCRASQSVSSSYLAWYQQKPGQAPRLLIYGTSSRATGIPDRFS GSGSGTDFTLTISRLEPEDFAVYYCQQYGSSPTFGGGTKVEIK (SEQ ID NO: 252) and a variable heavy chain including the sequence:

(SEQ ID NO: 253)
QVCLVESGGGVVQPGKSLRLSCAASGFTFSSYGMHWVRQAPGKGLEW
VAVIWYGGSNKYYADSVKGRFTISRDNSKNTLYLQMNSLRAEDTAVY
YCARDGTGENYYYYVMDVWGQGTTVTVS.

In some instances, additional scFv based on the binding domains described herein and for use in a CAR can be prepared according to methods known in the art (see, for example, Bird et al., (1988) Science 242:423-426 and Huston et al., (1988) Proc. Natl. Acad. Sci. USA 85:5879-5883). ScFv molecules can be produced by linking VH and VL regions of an antibody together using flexible polypeptide linkers. If a short polypeptide linker is employed (e.g., between 5-10 amino acids) intrachain folding is prevented. Interchain folding is also required to bring the two variable regions together to form a functional epitope binding site. For examples of linker orientations and sizes see, e.g., Hollinger et al. 1993 Proc Natl Acad. Sci. U.S.A. 90:6444-6448, US 2005/0100543, US 2005/0175606, US 2007/0014794, and WO2006/020258 and WO2007/024715. More particularly, linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies. scFv are commonly used as the binding domains of CAR.

Other binding fragments, such as Fv, Fab, Fab′, F(ab′)2, can also be used within the CAR disclosed herein. Additional examples of antibody-based binding domain formats for use in a CAR include scFv-based grababodies and soluble VH domain antibodies. These antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

Functional variants include one or more residue additions or substitutions that do not substantially impact the physiological effects of the protein. Functional fragments include one or more deletions or truncations that do not substantially impact the physiological effects of the protein. A lack of substantial impact can be confirmed by observing experimentally comparable results in an activation study or a binding study. Functional variants and functional fragments of intracellular domains (e.g., intracellular signaling domains) transmit activation or inhibition signals comparable to a wild-type reference when in the activated state of the current disclosure. Functional variants and functional fragments of binding domains bind their cognate antigen or ligand at a level comparable to a wild-type reference.

In particular embodiments, a VL region in a binding domain of the present disclosure is derived from or based on a VL of an antibody disclosed herein and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the antibody disclosed herein. An insertion, deletion or substitution may be anywhere in the VL region, including at the amino- or carboxy-terminus or both ends of this region, provided that each CDR includes zero changes or at most one, two, or three changes and provided a binding domain containing the modified VL region can still specifically bind its target with an affinity similar to the wild type binding domain.

In particular embodiments, a binding domain VH region of the present disclosure can be derived from or based on a VH of an antibody disclosed herein and can contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of the antibody disclosed herein. An insertion, deletion or substitution may be anywhere in the VH region, including at the amino- or carboxy-terminus or both ends of this region, provided that each CDR includes zero changes or at most one, two, or three changes and provided a binding domain containing the modified VH region can still specifically bind its target with an affinity similar to the wild type binding domain.

In particular embodiments, a binding domain includes or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (VL) or to a heavy chain variable region (VH), or both, wherein each CDR includes zero changes or at most one, two, or three changes, from an antibody disclosed herein or fragment or derivative thereof that specifically binds to CD33.

(iii-b-ii) Spacer regions are used to create appropriate distances and/or flexibility from other CAR sub-components. As indicated, in particular embodiments, the length of a spacer region is customized for binding CD33-expressing cells and mediating destruction. In particular embodiments, a spacer region length can be selected based upon the location of a cellular marker epitope, affinity of a binding domain for the epitope, and/or the ability of the CD33-targeting agent to mediate cell destruction following CD33 binding.

Spacer regions typically include those having 10 to 250 amino acids, 10 to 200 amino acids, 10 to 150 amino acids, 10 to 100 amino acids, 10 to 50 amino acids, or 10 to 25 amino acids.

In particular embodiments, a spacer region is 5 amino acids, 8 amino acids, 10 amino acids, 12 amino acids, 14 amino acids, 20 amino acids, 21 amino acids, 26 amino acids, 27 amino acids, 45 amino acids, or 50 amino acids. These lengths qualify as short spacer regions.

In particular embodiments, a spacer region is 100 amino acids, 110 amino acids, 120 amino acids, 125 amino acids, 128 amino acids, 131 amino acids, 135 amino acids, 140 amino acids, 150 amino acids, 160 amino acids, or 170 amino acids. These lengths qualify as intermediate spacer regions.

Exemplary spacer regions include all or a portion of an immunoglobulin hinge region. An immunoglobulin hinge region may be a wild-type immunoglobulin hinge region or an altered wild-type immunoglobulin hinge region. In certain embodiments, an immunoglobulin hinge region is a human immunoglobulin hinge region. As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

An immunoglobulin hinge region may be an IgG, IgA, IgD, IgE, or IgM hinge region. An IgG hinge region may be an IgG1, IgG2, IgG3, or IgG4 hinge region. Sequences from IgG1, IgG2, IgG3, IgG4 or IgD can be used alone or in combination with all or a portion of a CH2 region; all or a portion of a CH3 region; or all or a portion of a CH2 region and all or a portion of a CH3 region.

In particular embodiments, the spacer is a short spacer including an IgG4 hinge region. In particular embodiments the short spacer is encoded by either of SEQ ID NOs: 10 or 11. In particular embodiments, the spacer is an intermediate spacer including an IgG4 hinge region and an IgG4 hinge CH3 region. In particular embodiments the intermediate spacer is encoded by SEQ ID NO: 12. In particular embodiments, the spacer is a long spacer including an IgG4 hinge region, an IgG4 CH3 region, and an IgG4 CH2 region. In particular embodiments the long spacer is encoded by SEQ ID NO: 13.

Other examples of hinge regions that can be used CAR described herein include the hinge region present in the extracellular regions of type 1 membrane proteins, such as CD8a, CD4, CD28 and CD7, which may be wild-type or variants thereof.

In particular embodiments, a spacer region includes a hinge region that includes a type II C-lectin interdomain (stalk) region or a cluster of differentiation (CD) molecule stalk region. A “stalk region” of a type II C-lectin or CD molecule refers to the portion of the extracellular domain (ECD) of the type II C-lectin or CD molecule that is located between the C-type lectin-like domain (CTLD; e.g., similar to CTLD of natural killer cell receptors) and the hydrophobic portion (transmembrane domain). For example, the ECD of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al., Immunity 10:15, 1999; for descriptions of other stalk regions, see also Beavil et al., Proc. Nat'l. Acad. Sci. USA 89:153, 1992; and Figdor et al., Nat. Rev. Immunol. 2:11, 2002). These type II C-lectin or CD molecules may also have junction amino acids (described below) between the stalk region and the transmembrane region or the CTLD. In another example, the 233 amino acid human NKG2A protein (GenBank Accession No. P26715.1) has a hydrophobic portion (transmembrane domain) ranging from amino acids 71-93 and an ECD ranging from amino acids 94-233. The CTLD includes amino acids 119-231 and the stalk region includes amino acids 99-116, which may be flanked by additional junction amino acids. Other type II C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

(iii-b-iii) Transmembrane Domains. As indicated, transmembrane domains within a CAR serve to connect the extracellular component and intracellular component through the cell membrane. The transmembrane domain can anchor the expressed molecule in the modified cell's membrane.

The transmembrane domain can be derived either from a natural and/or a synthetic source. When the source is natural, the transmembrane domain can be derived from any membrane-bound or transmembrane protein. Transmembrane domains can include at least the transmembrane region(s) of the α, β or ζ chain of a T-cell receptor, CD28, CD27, CD3 epsilon, CD45, CD4, CD5, CD8, CD9, CD16, CD22; CD33, CD37, CD64, CD80, CD86, CD134, CD137 and CD154. In particular embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7R a, ITGA1, VLA1, CD49a, ITGA4, IA4, CD49D, ITGA6, VLA-6, CD49f, ITGAD, CDI Id, ITGAE, CD103, ITGAL, CDI Ia, ITGAM, CDI Ib, ITGAX, CDI Ic, ITGB1, CD29, ITGB2, CD18, ITGB7, TNFR2, DNAM1(CD226), SLAMF4 (CD244, 2B4), CD84, CD96 (Tactile), CEACAM1, CRT AM, Ly9(CD229), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, or NKG2C. In particular embodiments, a variety of human hinges can be employed as well including the human Ig (immunoglobulin) hinge (e.g., an IgG4 hinge, an IgD hinge), a GS linker (e.g., a GS linker described herein), a KIR2DS2 hinge or a CD8a hinge.

In particular embodiments, a transmembrane domain has a three-dimensional structure that is thermodynamically stable in a cell membrane, and generally ranges in length from 15 to 30 amino acids. The structure of a transmembrane domain can include an a helix, a p barrel, a p sheet, a p helix, or any combination thereof.

A transmembrane domain can include one or more additional amino acids adjacent to the transmembrane region, e.g., one or more amino acid within the extracellular region of the CAR (e.g., up to 15 amino acids of the extracellular region) and/or one or more additional amino acids within the intracellular region of the CAR (e.g., up to 15 amino acids of the intracellular components). In one aspect, the transmembrane domain is from the same protein that the signaling domain, co-stimulatory domain or the hinge domain is derived from. In another aspect, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins to minimize interactions with other unintended members of the receptor complex. In particular embodiments, the transmembrane domain is encoded by the nucleic acid sequence encoding the CD28 transmembrane domain (SEQ ID NOs: 21-23). In particular embodiments, the transmembrane domain includes the amino acid sequence of the CD28 transmembrane domain (SEQ ID NOs: 24 and 25).

(iii-b-iv) Intracellular Effector Domains. The intracellular effector domains of a CAR are responsible for activation of the cell in which the CAR is expressed. The term “effector domain” is thus meant to include any portion of the intracellular domain sufficient to transduce an activation signal. An effector domain can directly or indirectly promote a biological or physiological response in a cell when receiving the appropriate signal. In certain embodiments, an effector domain is part of a protein or protein complex that receives a signal when bound, or it binds directly to a target molecule, which triggers a signal from the effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a cancer cell. Activation of the modified cell can include one or more of differentiation, proliferation and/or activation or other effector functions. In particular embodiments, an effector domain can include an intracellular signaling component including a T cell receptor and a co-stimulatory domain which can include the cytoplasmic sequence from co-receptor or co-stimulatory molecule.

An effector domain can include one, two, three or more intracellular signaling components (e.g., receptor signaling domains, cytoplasmic signaling sequences), co-stimulatory domains, or combinations thereof. Exemplary effector domains include signaling and stimulatory domains selected from: 4-1BB (CD137), CARD11, CD3γ, CD3δ, CD3ε, CD3ζ, CD27, CD28, CD79A, CD79B, DAP10, FcRα, FcRβ (FcεR1b), FcRγ, Fyn, HVEM (LIGHTR), ICOS, LAG3, LAT, Lck, LRP, NKG2D, NOTCH1, pTα, PTCH2, OX40, ROR2, Ryk, SLAMF1, Slp76, TCRα, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKp80 (KLRF1), CD127, CD160, CD19, CD4, CD8a, CD8P, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, or NKp46.

Intracellular signaling component sequences that act in a stimulatory manner may include ITAMs. Examples of ITAMs including primary cytoplasmic signaling sequences include those derived from CD3γ, CD3δ, CD3ε, CD3ζ, CD5, CD22, CD66d, CD79a, CD79b, and common FcRγ (FCER1G), FcγRIIa, FcRβ (Fcε Rib), DAP10, and DAP12. In particular embodiments, variants of CD3ζ retain at least one, two, three, or all ITAM regions.

In particular embodiments, an effector domain includes a cytoplasmic portion that associates with a cytoplasmic signaling protein, wherein the cytoplasmic signaling protein is a lymphocyte receptor or signaling domain thereof, a protein including a plurality of ITAMs, a co-stimulatory domain, or any combination thereof.

Additional examples of intracellular signaling components include the cytoplasmic sequences of the CD3ζ chain, and/or co-receptors that act in concert to initiate signal transduction following binding domain engagement.

A co-stimulatory domain is a domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83. For example, CD27 co-stimulation has been demonstrated to enhance expansion, effector function, and survival of human CART cells in vitro and augments human T cell persistence and anti-cancer activity in vivo (Song et al. Blood. 2012; 119(3):696-706). Further examples of such co-stimulatory domain molecules include CDS, ICAM-1, GITR, BAFFR, HVEM (LIGHTR), SLAMF7, NKp80 (KLRF1), NKp44, NKp30, NKp46, CD160, CD19, CD4, CD8a, CD8P, IL2Rβ, IL2Rγ, IL7Rα, ITGA4, VLA1, CD49a, ITGA4, IA4, CD49D, ITGA6, VLA-6, CD49f, ITGAD, CDIId, ITGAE, CD103, ITGAL, CDIIa, ITGAM, CDI Ib, ITGAX, CDIIc, ITGBI, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

In particular embodiments, the nucleic acid sequences encoding the intracellular signaling components includes CD3z encoding sequence (SEQ ID NO: 14) and a variant of the 4-1EE signaling encoding sequence (SEQ ID NOs: 17 and 18). In particular embodiments, the amino acid sequence of the intracellular signaling component includes a variant of CD3ζ (SEQ ID NOs: 15 and 16) and a portion of the 4-1EE (SEQ ID NO: 19 and 20) intracellular signaling component.

In particular embodiments, the intracellular signaling component includes (i) all or a portion of the signaling domain of CD3ζ, (ii) all or a portion of the signaling domain of 4-1BE, or (iii) all or a portion of the signaling domain of CD3ζ and 4-1BB. In particular embodiments, the intracellular signaling component includes (i) all or a portion of the signaling domain of CD3ζ, (ii) all or a portion of the signaling domain of 4-1 BB, (iii) all or a portion of the signaling domain of CD28, (iv) or all or a portion of the signaling domain of CD3ζ, 4-1BB, and CD28.

Intracellular components may also include one or more of a protein of a Wnt signaling pathway (e.g., LRP, Ryk, or ROR2), NOTCH signaling pathway (e.g., NOTCH1, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family); G-protein-coupled receptors, GPCRs (Frizzled or Smoothened); serine/threonine kinase receptors (BMPR or TGFR); or cytokine receptors (IL1R, IL2R, IL7R, or IL15R).

(iii-b-v) Linkers. As used herein, a linker can include any portion of a CAR molecule that serves to connect two other subcomponents of the molecule. Some linkers serve no purpose other than to link components while many linkers serve an additional purpose. Linkers can, for example, link VL and VH of antibody derived binding domains of scFvs and serve as junction amino acids between subcomponent portions of a CAR.

Linkers can be flexible, rigid, or semi-rigid, depending on the desired function of the linker. Linkers can include junction amino acids. For example, in particular embodiments, linkers provide flexibility and room for conformational movement between different components of CAR. Commonly used flexible linkers include Gly-Ser linkers. In particular embodiments, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include (Gly4Ser)n (SEQ ID NO: 160), (Gly3Ser)n(Gly4Ser)n (SEQ ID NO: 161), (Gly3Ser)n(Gly2Ser)n (SEQ ID NO: 162), or (Gly3Ser)n(Gly4Ser)1 (SEQ ID NO: 163). In particular embodiments, the linker is (Gly4Ser)4 (SEQ ID NO: 164), (Gly4Ser)3 (SEQ ID NO: 165), (Gly4Ser)2 (SEQ ID NO: 166), (Gly4Ser)1 (SEQ ID NO: 167), (Gly3Ser)2 (SEQ ID NO: 168), (Gly3Ser)1 (SEQ ID NO: 169), (Gly2Ser)2 (SEQ ID NO: 170) or (Gly2Ser)1, GGSGGGSGGSG (SEQ ID NO: 171), GGSGGGSGSG (SEQ ID NO: 172), or GGSGGGSG (SEQ ID NO: 173).

In particular embodiments, a linker region is (GGGGS)n (SEQ ID NO: 160) wherein n is an integer including, 1, 2, 3, 4, 5, 6, 7, 8, 9, or more. In particular embodiments, the spacer region is (EAAAK)n (SEQ ID NO: 174) wherein n is an integer including 1, 2, 3, 4, 5, 6, 7, 8, 9, or more.

In some situations, flexible linkers may be incapable of maintaining a distance or positioning of CAR needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

Linkers can be susceptible to cleavage (cleavable linker), such as, acid-induced cleavage, photo-induced cleavage, peptidase-induced cleavage, esterase-induced cleavage, and disulfide bond cleavage. Alternatively, linkers can be substantially resistant to cleavage (e.g., stable linker or noncleavable linker). In some aspects, the linker is a procharged linker, a hydrophilic linker, or a dicarboxylic acid-based linker.

Junction amino acids can be a linker which can be used to connect sequences when the distance provided by a spacer region is not needed and/or wanted. For example, junction amino acids can be short amino acid sequences that can be used to connect co-stimulatory intracellular signaling components. In particular embodiments, junction amino acids are 9 amino acids or less (e.g., 2, 3, 4, 5, 6, 7, 8, or 9 amino acids). In particular embodiments, a glycine-serine doublet can be used as a suitable junction amino acid linker. In particular embodiments, a single amino acid, e.g., an alanine, a glycine, can be used as a suitable junction amino acid.

(iii-b-vi) Control Features Including Tag Cassettes, Transduction Markers, and/or Suicide Switches. In particular embodiments, CAR constructs can include one or more tag cassettes and/or transduction markers. Tag cassettes and transduction markers can be used to activate, promote proliferation of, detect, enrich for, isolate, track, deplete and/or eliminate genetically modified cells in vitro, in vivo and/or ex vivo. “Tag cassette” refers to a unique synthetic peptide sequence affixed to, fused to, or that is part of a CAR, to which a cognate binding molecule (e.g., ligand, antibody, or other binding partner) is capable of specifically binding where the binding property can be used to activate, promote proliferation of, detect, enrich for, isolate, track, deplete and/or eliminate the tagged protein and/or cells expressing the tagged protein. Transduction markers can serve the same purposes but are derived from naturally occurring molecules and are often expressed using a skipping element that separates the transduction marker from the rest of the CAR molecule.

Tag cassettes that bind cognate binding molecules include, for example, His tag (HHHHHH; SEQ ID NO: 175), Flag tag (DYKDDDDK; SEQ ID NO: 176), Xpress tag (DLYDDDDK; SEQ ID NO: 177), Avi tag (GLNDIFEAQKIEWHE; SEQ ID NO: 178), Calmodulin tag (KRRWKKNFIAVSAANRFKKISSSGAL; SEQ ID NO: 179), Polyglutamate tag, HA tag (YPYDVPDYA; SEQ ID NO: 180), Myc tag (EQKLISEEDL; SEQ ID NO: 181), Strep tag (which refers the original STREP® tag (WRHPQFGG; SEQ ID NO: 182), STREP® tag II (WSHPQFEK SEQ ID NO: 183 (IBA Institut fur Bioanalytik, Germany); see, e.g., U.S. Pat. No. 7,981,632), Softag 1 (SLAELLNAGLGGS; SEQ ID NO: 184), Softag 3 (TQDPSRVG; SEQ ID NO: 185), and V5 tag (GKPIPNPLLGLDST; SEQ ID NO: 186).

Conjugate binding molecules that specifically bind tag cassette sequences disclosed herein are commercially available. For example, His tag antibodies are commercially available from suppliers including Life Technologies, Pierce Antibodies, and GenScript.Flag tag antibodies are commercially available from suppliers including Pierce Antibodies, GenScript, and Sigma-Aldrich. Xpress tag antibodies are commercially available from suppliers including Pierce Antibodies, Life Technologies and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal and Abcam. Myc tag antibodies are commercially available from suppliers including Santa Cruz Biotechnology, Abcam, and Cell Signal. Strep tag antibodies are commercially available from suppliers including Abcam, Iba, and Qiagen.

Transduction markers may be selected from at least one of a truncated CD19 (tCD19; see Budde et al., Blood 122: 1660, 2013); a truncated human EGFR (tEGFR; see Wang et al., Blood 118: 1255, 2011); an extracellular domain of human CD34; and/or RQR8 which combines target epitopes from CD34 (see Fehse et al, Mol. Therapy 1(5 Pt 1); 448-456, 2000) and CD20 antigens (see Philip et al, Blood 124: 1277-1278).

In particular embodiments, a polynucleotide encoding an iCaspase9 construct (iCasp9) may be inserted into a CAR construct as a suicide switch.

Control features may be present in multiple copies in a CAR or can be expressed as distinct molecules with the use of a skipping element (SEQ ID NOs: 187). For example, a CAR can have one, two, three, four or five tag cassettes and/or one, two, three, four, or five transduction markers could also be expressed. For example, embodiments can include a CAR construct having two Myc tag cassettes, or a His tag and an HA tag cassette, or a HA tag and a Softag 1 tag cassette, or a Myc tag and a SBP tag cassette. Exemplary transduction markers and cognate pairs are described in U.S. Ser. No. 13/463,247.

One advantage of including at least one control feature in a CAR is that cells expressing CAR administered to a subject can be increased or depleted using the cognate binding molecule to a tag cassette. In certain embodiments, the present disclosure provides a method for depleting a modified cell expressing a CAR by using an antibody specific for the tag cassette, using a cognate binding molecule specific for the control feature, or by using a second modified cell expressing a CAR and having specificity for the control feature. Elimination of modified cells may be accomplished using depletion agents specific for a control feature. For example, if tEGFR is used, then an anti-tEGFR binding domain (e.g., antibody, scFv) fused to or conjugated to a cell-toxic reagent (such as a toxin, radiometal) may be used, or an anti-tEGFR/anti-CD3 bispecific scFv, or an anti-tEGFR CAR T cell may be used.

In certain embodiments, modified cells expressing a chimeric molecule may be detected or tracked in vivo by using antibodies that bind with specificity to a control feature (e.g., anti-Tag antibodies), or by other cognate binding molecules that specifically bind the control feature, which binding partners for the control feature are conjugated to a fluorescent dye, radio-tracer, iron-oxide nanoparticle or other imaging agent known in the art for detection by X-ray, CT-scan, MRI-scan, PET-scan, ultrasound, flow-cytometry, near infrared imaging systems, or other imaging modalities (see, e.g., Yu, et al., Theranostics 2:3, 2012).

Thus, modified cells expressing at least one control feature with a CAR can be, e.g., more readily identified, isolated, sorted, induced to proliferate, tracked, and/or eliminated as compared to a modified cell without a tag cassette.

### (iv) CELL ACTIVATING CULTURE CONDITIONS

Cell populations can be incubated in a culture-initiating composition to expand genetically modified cell populations. The incubation can be carried out in a culture vessel, such as a bag, cell culture plate, flask, chamber, chromatography column, cross-linked gel, cross-linked polymer, column, culture dish, hollow fiber, microtiter plate, silica-coated glass plate, tube, tubing set, well, vial, or other container for culture or cultivating cells.

Culture conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177, Klebanoff et al. (2012) J Immunother. 35(9): 651-660, Terakura et al. (2012) Blood. 1:72-82, and/or Wang et al. (2012) J Immunother. 35(9):689-701.

Exemplary culture media for culturing T cells include (i) RPMI supplemented with non-essential amino acids, sodium pyruvate, and penicillin/streptomycin; (ii) RPMI with HEPES, 5-15% human serum, 1-3% L-Glutamine, 0.5-1.5% penicillin/streptomycin, and 0.25×10-4-0.75×10-4 M β-MercaptoEthanol; (iii) RPMI-1640 supplemented with 10% fetal bovine serum (FBS), 2 mM L-glutamine, 10 mM HEPES, 100 U/ml penicillin and 100 m/mL streptomycin; (iv) DMEM medium supplemented with 10% FBS, 2 mM L-glutamine, 10 mM HEPES, 100 U/ml penicillin and 100 m/mL streptomycin; and (v) X-Vivo 15 medium (Lonza, Walkersville, MD) supplemented with 5% human AB serum (Gemcell, West Sacramento, CA), 1% HEPES (Gibco, Grand Island, NY), 1% Pen-Strep (Gibco), 1% GlutaMax (Gibco), and 2% N-acetyl cysteine (Sigma-Aldrich, St. Louis, MO). T cell culture media are also commercially available from Hyclone (Logan, UT). Additional T cell activating components that can be added to such culture media are described in more detail below.

In some embodiments, the T cells are expanded by adding to the culture-initiating composition feeder cells, such as non-dividing peripheral blood mononuclear cells (PBMC), (e.g., such that the resulting population of cells contains at least 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can include gamma-irradiated PBMC feeder cells. In some embodiments, the PBMC are irradiated with gamma rays in the range of 3000 to 3600 rads to prevent cell division. In some aspects, the feeder cells are added to culture medium prior to the addition of the populations of T cells.

Optionally, the incubation may further include adding non-dividing EBV-transformed lymphoblastoid cells (LCL) as feeder cells. LCL can be irradiated with gamma rays in the range of 6000 to 10,000 rads. The LCL feeder cells in some aspects is provided in any suitable amount, such as a ratio of LCL feeder cells to initial T lymphocytes of at least 10:1.

In some embodiments, the stimulating conditions include temperature suitable for the growth of human T lymphocytes, for example, at least 25° C., at least 30° C., or 37° C.

The activating culture conditions for T cells include conditions whereby T cells of the culture-initiating composition proliferate or expand. T cell activating conditions can include one or more cytokines, for example, interleukin (IL)-2, IL-7, IL-15 and/or IL-21. IL-2 can be included at a range of 10-100 ng/mL (e.g., 40, 50, or 60 ng/mL). IL-7, IL-15, and/or IL-21 can be individually included at a range of 0.1-50 ng/mL (e.g., 5, 10, or 15 ng/mL). Particular embodiments utilize IL-2 at 50 ng/mL. Particular embodiments utilize IL-7, IL-15 and IL-21 individually included at 10 ng/mL.

In particular embodiments, T cell activating culture condition conditions can include T cell stimulating epitopes. T cell stimulating epitopes include CD3, CD27, CD2, CD4, CD5, CD7, CD8, CD28, CD30, CD40, CD56, CD83, CD90, CD95, 4-1BB (CD 137), B7-H3, CTLA-4, Frizzled-1 (FZD1), FZD2, FZD3, FZD4, FZD5, FZD6, FZD7, FZD8, FZD9, FZD10, HVEM, ICOS, IL-1R, LAT, LFA-1, LIGHT, MHCI, MHCII, NKG2D, OX40, ROR2 and RTK.

CD3 is a primary signal transduction element of T cell receptors. As indicated previously, CD3 is expressed on all mature T cells. In particular embodiments, the CD3 stimulating molecule (i.e., CD3 binding domain) can be derived from the OKT3 antibody (see U.S. Pat. Nos. 5,929,212; 4,361,549; ATCC® CRL-8001™; and Arakawa et al., J. Biochem. 120, 657-662 (1996)), the 20G6-F3 antibody, the 4B4-D7 antibody, the 4E7-C9, or the 18F5-H10 antibody.

In particular embodiments, CD3 stimulating molecules can be included within culture media at a concentration of at least 0.25 or 0.5 ng/ml or at a concentration of 2.5-10 μg/mL. Particular embodiments utilize a CD3 stimulating molecule (e.g., OKT3) at 5 μg/mL.

In particular embodiments, activating molecules associated with avi-tags can be biotinylated and bound to streptavidin beads. This approach can be used to create, for example, a removable T cell epitope stimulating activation system.

An exemplary binding domain for CD28 can include or be derived from TGN1412, CD80, CD86 or the 9D7 antibody. Additional antibodies that bind CD28 include 9.3, KOLT-2, 15E8, 248.23.2, EX5.3D10, and CD28.3 (deposited as a synthetic single chain Fv construct under GenBank Accession No. AF451974.1; see also Vanhove et al., BLOOD, 15 Jul. 2003, Vol. 102, No. 2, pages 564-570). Further, 1YJD provides a crystal structure of human CD28 in complex with the Fab fragment of a mitogenic antibody (5.11A1). In particular embodiments, antibodies that do not compete with 9D7 are selected.

4-1BB binding domains can be derived from LOB12, IgG2a, LOB12.3, or IgG1 as described in Taraban et al. Eur J Immunol. 2002 December; 32(12):3617-27. In particular embodiments a 4-1BB binding domain is derived from a monoclonal antibody described in U.S. Pat. No. 9,382,328. Additional 4-1BB binding domains are described in U.S. Pat. Nos. 6,569,997, 6,303,121, and Mittler et al. Immunol Res. 2004; 29(1-3):197-208.

OX40 (CD134) and/or ICOS activation may also be used. OX40 binding domains are described in US20100196359, US 20150307617, WO 2015/153513, WO2013/038191 and Melero et al. Clin Cancer Res. 2013 Mar. 1; 19(5):1044-53. Exemplary binding domains that can bind and activate ICOS are described in e.g., US20080279851 and Deng et al. Hybrid Hybridomics. 2004 June; 23(3):176-82.

When in soluble form, T-cell activating agents can be coupled with another molecule, such as polyethylene glycol (PEG) molecule. Any suitable PEG molecule can be used. Typically, PEG molecules up to a molecular weight of 1000 Da are soluble in water or culture media. In some cases, such PEG based reagent can be prepared using commercially available activated PEG molecules (for example, PEG-NHS derivatives available from NOF North America Corporation, Irvine, Calif., USA, or activated PEG derivatives available from Creative PEGWorks, Chapel Hills, N.C., USA).

In particular embodiments, cell stimulating agents are immobilized on a solid phase within the culture media. In particular embodiments, the solid phase is a surface of the culture vessel (e.g., bag, cell culture plate, chamber, chromatography column, cross-linked gel, cross-linked polymer, column, culture dish, hollow fiber, microtiter plate, silica-coated glass plate, tube, tubing set, well, vial, other structure or container for culture or cultivation of cells).

In particular embodiments, a solid phase can be added to a culture media. Such solid phases can include, for example, beads, hollow fibers, resins, membranes, and polymers.

Exemplary beads include magnetic beads, polymeric beads, and resin beads (e.g., Strep-Tactin® Sepharose, Strep-Tactin® Superflow, and Strep-Tactin® MacroPrep IBA GmbH, Gottingen)). Anti-CD3/anti-CD28 beads are commercially available reagents for T cell expansion (Invitrogen). These beads are uniform, 4.5 μm superparamagnetic, sterile, non-pyrogenic polystyrene beads coated with a mixture of affinity purified monoclonal antibodies against the CD3 and CD28 cell surface molecules on human T cells. Hollow fibers are available from TerumoBCT Inc. (Lakewood, Colo., USA). Resins include metal affinity chromatography (IMAC) resins (e.g., TALON® resins (Westburg, Leusden)). Membranes include paper as well as the membrane substrate of a chromatography matrix (e.g., a nitrocellulose membrane or a polyvinylidene difluoride (PVDF) membrane).

Exemplary polymers include polysaccharides, such as polysaccharide matrices. Such matrices include agarose gels (e.g., Superflow™ agarose or a Sepharose® material such as Superflow™ Sepharose® that are commercially available in different bead and pore sizes) or a gel of crosslinked dextran(s). A further illustrative example is a particulate cross-linked agarose matrix, to which dextran is covalently bonded, that is commercially available (in various bead sizes and with various pore sizes) as Sephadex® or Superdex®, both available from GE Healthcare.

Synthetic polymers that may be used include polyacrylamide, polymethacrylate, a copolymer of polysaccharide and agarose (e.g. a polyacrylamide/agarose composite) or a polysaccharide and N,N′-methylenebisacrylamide. An example of a copolymer of a dextran and N,N′-methylenebisacrylamide is the Sephacryl® (Pharmacia Fine Chemicals, Inc., Piscataway, NJ) series of materials.

Particular embodiments may utilize silica particles coupled to a synthetic or to a natural polymer, such as polysaccharide grafted silica, polyvinylpyrrolidone grafted silica, polyethylene oxide grafted silica, poly(2-hydroxyethylaspartamide) silica and poly(N-isopropylacrylamide) grafted silica.

Cell activating agents can be immobilized to solid phases through covalent bonds or can be reversibly immobilized through non-covalent attachments.

In particular embodiments, a T-cell activating culture media includes a FACS-sorted T cell population cultured within RPMI with HEPES, 5-15% human serum, 1-3% L-Glutamine, 0.5-1.5% Pen/strep, 0.25×10-4-0.75×10⁻⁴ M β-MercaptoEthanol, with IL-7, IL-15 and IL-21 individually included at 5-15 (e.g., 10) ng/μl. The culture is carried out on a flat-bottom well plate with 0.1-0.5×10e6 plated cells/well. On Day 3 post activation cells are transferred to a TC-treated plate.

In particular embodiments, a T-cell activating culture media includes a FACS-sorted CD8+T population cultured within RPMI with HEPES, 10% human serum, 2% L-Glutamine, 1% Pen/strep, 0.5×10⁻⁴ M β-MercaptoEthanol, with IL-7, IL-15 and IL-21 individually included at 5-15 (e.g., 10) ng/μl. The culture is carried out on a flat-bottom non-tissue culture (TC)-treated 96/48-well plate with 0.1-0.5×10e6 plated cells/well. On Day 3 post activation cells are transferred to TC-treated plate.

Culture conditions for HSC/HSP can include expansion with a Notch agonist (see, e.g., U.S. Pat. Nos. 7,399,633; 5,780,300; 5,648,464; 5,849,869; and 5,856,441 and growth factors present in the culture condition as follows: 25-300 ng/ml SCF, 25-300 ng/ml Flt-3L, 25-100 ng/ml TPO, 25-100 ng/ml IL-6 and 10 ng/ml IL-3. In more specific embodiments, 50, 100, or 200 ng/ml SCF; 50, 100, or 200 ng/ml of Flt-3L; 50 or 100 ng/ml TPO; 50 or 100 ng/ml IL-6; and 10 ng/ml IL-3 can be used.

### (v) EX VIVO MANUFACTURED CELL FORMULATIONS

In particular embodiments, genetically modified cells can be harvested from a culture medium and washed and concentrated into a carrier in a therapeutically-effective amount. Exemplary carriers include saline, buffered saline, physiological saline, water, Hanks' solution, Ringer's solution, Nonnosol-R (Abbott Labs), PLASMA-LYTE A® (Baxter Laboratories, Inc., Morton Grove, IL), glycerol, ethanol, and combinations thereof.

In particular embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, compositions or formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within compositions or formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

In compositions and formulations disclosed herein, cells are generally in a volume of a liter or less, 500 mls or less, 250 mls or less or 100 mls or less. Hence the density of administered cells is typically greater than 10⁴ cells/ml, 10⁷ cells/ml or 10⁸ cells/ml.

As indicated, compositions include at least one genetically modified cell type (e.g., modified T cells, NK cells, or stem cells). Formulations can include different types of genetically modified cells (e.g., T cells, NK cells, and/or stem cells in combination).

Different types of genetically-modified cells or cell subsets (e.g., modified T cells, NK cells, and/or stem cells) can be provided in different ratios e.g., a 1:1:1 ratio, 2:1:1 ratio, 1:2:1 ratio, 1:1:2 ratio, 5:1:1 ratio, 1:5:1 ratio, 1:1:5 ratio, 10:1:1 ratio, 1:10:1 ratio, 1:1:10 ratio, 2:2:1 ratio, 1:2:2 ratio, 2:1:2 ratio, 5:5:1 ratio, 1:5:5 ratio, 5:1:5 ratio, 10:10:1 ratio, 1:10:10 ratio, 10:1:10 ratio, etc. These ratios can also apply to numbers of cells expressing the same or different CAR components. If only two of the cell types are combined or only 2 combinations of expressed CAR components are included within a formulation, the ratio can include any 2-number combination that can be created from the 3 number combinations provided above. In embodiments, the combined cell populations are tested for efficacy and/or cell proliferation in vitro, in vivo and/or ex vivo, and the ratio of cells that provides for efficacy and/or proliferation of cells is selected. Particular embodiments include a 1:1 ratio of CD4 T cells and CD8 T cells.

The cell-based compositions disclosed herein can be prepared for administration by, e.g., injection, infusion, perfusion, or lavage. The compositions and formulations can further be formulated for bone marrow, intravenous, intradermal, intraarterial, intranodal, intralymphatic, intraperitoneal, intralesional, intratumoral, intravesicular, and/or subcutaneous injection.

### (vi) METHODS OF USE

Methods disclosed herein include treating subjects (humans, veterinary animals (dogs, cats, reptiles, birds, etc.) livestock (horses, cattle, goats, pigs, chickens, etc.) and research animals (monkeys, rats, mice, fish, etc.) with compositions and formulations disclosed herein. Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological change in the subject. For example, an effective amount can provide an immunogenic anti-cancer effect. Effective amounts are often administered for research purposes. Effective amounts disclosed herein can cause a statistically significant effect in an animal model or in vitro assay relevant to the assessment of a cancer's development or progression. An immunogenic composition can be provided in an effective amount, wherein the effective amount stimulates an immune response.

A “prophylactic treatment” includes a treatment administered to a subject who does not display signs or symptoms of a cancer or displays only early signs or symptoms of a cancer such that treatment is administered for the purpose of diminishing or decreasing the risk of developing the cancer further. Thus, a prophylactic treatment functions as a preventative treatment against a CD33-expressing cancer. In particular embodiments, prophylactic treatments reduce, delay, or prevent metastasis from a primary a cancer tumor site from occurring.

A “therapeutic treatment” includes a treatment administered to a subject who displays symptoms or signs of a cancer and is administered to the subject for the purpose of diminishing or eliminating those signs or symptoms of the cancer. The therapeutic treatment can reduce, control, or eliminate the presence or activity of the cancer and/or reduce control or eliminate side effects of the cancer.

Function as an effective amount, prophylactic treatment or therapeutic treatment are not mutually exclusive, and in particular embodiments, administered dosages may accomplish more than one treatment type.

In particular embodiments, therapeutically effective amounts provide anti-cancer effects. Anti-cancer effects include a decrease in the number of cancer cells, decrease in the number of metastases, a decrease in tumor volume, an increase in life expectancy, induced chemo- or radiosensitivity in cancer cells, inhibited angiogenesis near cancer cells, inhibited cancer cell proliferation, inhibited tumor growth, prevented or reduced metastases, prolonged subject life, reduced cancer-associated pain, and/or reduced relapse or re-occurrence of cancer following treatment.

A “tumor” is a swelling or lesion formed by an abnormal growth of cells (called neoplastic cells or tumor cells). A “tumor cell” is an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be benign, pre-malignant or malignant.

In particular embodiments, therapeutically effective amounts induce an immune response. The immune response can be against a cancer cell.

Examples of CD33-related disorders include hematological cancers such as leukemias and lymphomas and other myelo- or lymphoproliferative disorders.

Exemplary leukemias include acute lymphoblastic leukemia (ALL), AML, chronic myelogenous leukemia (CML), chronic myelomonocytic leukemia (CML), mast cell leukemia, myelodysplastic syndrome (MDS), B-cell acute lymphoblastic leukemia (B-ALL), T-cell acute lymphoblastic leukemia (T-ALL), and megakaryocytic leukemia.

Exemplary sub-types of AML include: acute basophilic leukemia, acute erythroid leukemia (AML-M6), acute megakaryoblastic leukemia (AML-M7), acute monoblastic leukemia (AML-M5a), acute monocytic leukemia (AML-M5b), acute myeloblasts leukemia with granulocytic maturation, acute myeloblasts leukemia without maturation, acute myelomonocytic leukemia (AML-M4), acute panmyelosis with myelofibrosis, acute promyelocytic leukemia (APL), erythroleukemia (AML-M6a), minimally differentiated acute myeloblasts leukemia, myelomonocytic leukemia with bone marrow eosinophilia, and pure erythroid leukemia (AML-M6b).

An exemplary lymphoma includes multiple myeloma.

Compositions disclosed herein can also be used to treat a complication or disease related to the above-noted lymphoproliferative disorders and hematological cancers. For example, complications relating to AML may include a preceding myelodysplastic syndrome (MDS, formerly known as “preleukemia”), secondary leukemia, in particular secondary AML, high white blood cell count, and absence of Auer rods. Among others, leukostasis and involvement of the central nervous system (CNS), hyperleukocytosis, residual disease, are also considered complications or diseases related to AML.

Compositions disclosed herein can be used to target myeloid-derived suppressor cells (MDSCs). MDSCs are a major player in the immunosuppressive tumor microenvironment and have been found to inhibit the antitumor reactivity of T cells and NK cells. Particular MDSCs have high CD33 expression and can be targeted with anti-CD33 treatments, including monocytic MDSCs and immature MDSCs.

Compositions disclosed herein may also find use in the treatment of other pathological conditions or genetic syndromes associated with the risk of AML such as Down syndrome, trisomy, Fanconi anemia, Bloom syndrome, Ataxia-telangiectasia, Diamond-Blackfan anemia, Schwachman-Diamond syndrome, Li-Fraumeni syndrome, Neurofibromatosis type 1, Severe congenital neutropenia (also called Kostmann syndrome).

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of cancer, stage of cancer, previous or concurrent therapeutic interventions, idiopathy of the subject and route of administration.

Therapeutically effective amounts of cell-based compositions can include 10⁴ to 10⁹ cells/kg body weight, or 10³ to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

Therapeutically effective amounts can be administered by, e.g., injection, infusion, perfusion, or lavage. Routes of administration can include bolus intravenous, intradermal, intraarterial, intraparenteral, intranodal, intralymphatic, intraperitoneal, intralesional, intraprostatic, intrathecal, intratumoral, intravesicular, and/or subcutaneous.

In certain embodiments, cells are administered to a patient in conjunction with (e.g., before, simultaneously or following) any number of relevant treatment modalities. In particular embodiments, cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycoplienolic acid, steroids, FR901228, cytokines, and irradiation.

### (vii) REFERENCE LEVELS DERIVED FROM CONTROL POPULATIONS

Obtained values for parameters associated with a therapy described herein can be compared to a reference level derived from a control population, and this comparison can indicate whether a therapy described herein is effective for a subject in need thereof. Reference levels can be obtained from one or more relevant datasets from a control population. A “dataset” as used herein is a set of numerical values resulting from evaluation of a sample (or population of samples) under a desired condition. The values of the dataset can be obtained, for example, by experimentally obtaining measures from a sample and constructing a dataset from these measurements. As is understood by one of ordinary skill in the art, the reference level can be based on e.g., any mathematical or statistical formula useful and known in the art for arriving at a meaningful aggregate reference level from a collection of individual data points; e.g., mean, median, median of the mean, etc. Alternatively, a reference level or dataset to create a reference level can be obtained from a service provider such as a laboratory, or from a database or a server on which the dataset has been stored.

A reference level from a dataset can be derived from previous measures derived from a control population. A “control population” is any grouping of subjects or samples of like specified characteristics. The grouping could be according to, for example, clinical parameters, clinical assessments, therapeutic regimens, disease status, severity of condition, etc. In particular embodiments, the grouping is based on age range (e.g., 60-65 years) and non-immunocompromised status. In particular embodiments, a normal control population includes individuals that are age-matched to a test subject and non-immune compromised. In particular embodiments, age-matched includes, e.g., 0-10 years old; 30-40 years old, 60-65 years old, 70-85 years old, etc., as is clinically relevant under the circumstances. In particular embodiments, a control population can include those that have a CD33-related disorder and have not been administered a therapeutically effective amount

In particular embodiments, the relevant reference level for values of a particular parameter associated with a therapy described herein is obtained based on the value of a particular corresponding parameter associated with a therapy in a control population to determine whether a therapy disclosed herein has been therapeutically effective for a subject in need thereof.

In particular embodiments, conclusions are drawn based on whether a sample value is statistically significantly different or not statistically significantly different from a reference level. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular data point, where the data point is the result of random chance alone. A result is often considered significant (not random chance) at a p-value less than or equal to 0.05. In particular embodiments, a sample value is “comparable to” a reference level derived from a normal control population if the sample value and the reference level are not statistically significantly different.

### (VIII) EXEMPLARY EMBODIMENTS

1. A chimeric antigen receptor (CAR) including an extracellular component including a binding domain having a complementarity determining region (CDR) set of antibody 1H10, 1A9, 1E6, 1D2, 1B9, 1H8, 2D3, or 2E3, according to North, IMGT, Kabat, Chothia or Set 5;
an intracellular component including an effector domain; and
a transmembrane domain linking the extracellular component to the intracellular component.

2. A CAR of embodiment 1, wherein the binding domain includes a single chain variable fragment (scFv).

3. A CAR of embodiment 2, wherein the scFv is encoded by the 1H10 scFv coding sequence as set forth in SEQ ID NO: 2;
the 1A9 scFv coding sequence as set forth in SEQ ID NO: 3;
the 1E6 scFv coding sequence as set forth in SEQ ID NO: 4;
the 1 D2 scFv coding sequence as set forth in SEQ ID NO: 5;
the 1B9 scFv coding sequence as set forth in SEQ ID NO: 6;
the 1H8 scFv coding sequence as set forth in SEQ ID NO: 7;
the 2D3 scFv coding sequence as set forth in SEQ ID NO: 8; or
the 2E3 scFv coding sequence as set forth in SEQ ID NO: 9.

4. A CAR of embodiment 2, wherein the scFv is the 1H10 scFv in the VH-VL orientation as set forth in SEQ ID NO: 190;
the 1H10 scFv in the VL-VH orientation as set forth in SEQ ID NO: 191;
the 1A9 scFv in the VH-VL orientation as set forth in SEQ ID NO: 192;
the 1A9 scFv in the VL-VH orientation as set forth in SEQ ID NO: 193;
the 1E6 scFv in the VH-VL orientation as set forth in SEQ ID NO: 194;
the 1E6 scFv in the VL-VH orientation as set forth in SEQ ID NO: 195;
the 2D3 scFv in the VH-VL orientation as set forth in SEQ ID NO: 196; or
the 2D3 scFv in the VL-VH orientation as set forth in SEQ ID NO: 197.

5. A CAR of any of embodiments 1-4, wherein the extracellular component further includes a spacer region.

6. A CAR of embodiment 5, wherein the spacer region is 135 amino acids or less or 16 amino acids of less.

7. A CAR of embodiments 5 or 6, wherein the spacer region is 131 amino acids or less and includes the hinge region and CH3 domain of IgG4.

8. A CAR of embodiments 5 or 6, wherein the spacer region is 12 amino acids or less and includes the hinge region of IgG4.

9. A CAR of embodiments 7 or 8 wherein the IgG4 is human IgG4.

10. A CAR of any of embodiments 5-8, wherein the spacer region is encoded by the IgG4 hinge coding sequence-A as set forth in SEQ ID NO: 10; the IgG4 hinge coding sequence-B as set forth in SEQ ID NO: 11; or the IgG4-int(DS) coding sequence as set forth in SEQ ID NO: 12.

11. A CAR of any of embodiments 1-10, wherein the effector domain includes all or a portion of the signaling domain of CD3ζ; all or a portion of the signaling domain of 4-1BB, all or a portion of the signaling domain of CD28, all or a portion of the signaling domain of CD3ζ and 4-1BB; all or a portion of the signaling domain of CD3ζ and CD28; or all or a portion of the signaling domain of CD3ζ, 4-1BB, and CD28

12. A CAR of any of embodiments 1-11, wherein the effector domain includes all or a portion of the signaling domain of CD3ζ and 4-1BB.

13. A CAR of embodiments 11 or 12, wherein the CD3ζ signaling domain is encoded by the CD3ζ coding sequence as set forth in SEQ ID NO: 14.

14. A CAR of any of embodiments 11-13, wherein the CD3ζ has the sequence as set forth in SEQ ID NO: 15 or SEQ ID NO: 16.

15. A CAR of any of embodiments 11-14, wherein the 4-1 BB signaling domain is encoded by 4-1BB signaling coding sequence-A as set forth in SEQ ID NO: 17 or 4-1 BB signaling coding sequence-B as set forth in SEQ ID NO: 18.

16. A CAR of any of embodiments 11-15, wherein the 4-1 BB has the sequence as set forth in SEQ ID NO: 19 or SEQ ID NO: 20.

17. A CAR of any of embodiments 1-16, wherein the transmembrane domain includes a CD28 transmembrane domain.

18. A CAR of embodiment 17, wherein the CD28 transmembrane domain is encoded by CD28TM coding sequence-A (SEQ ID NO: 21);
CD28TM coding sequence-B (SEQ ID NO: 22); or
or CD28TM coding sequence-C(SEQ ID NO: 23).

19. A CAR of embodiment 17, wherein the CD28 transmembrane domain has the sequence as set forth in SEQ ID NO: 24 or SEQ ID NO: 25.

20. A CAR of any of embodiments 1-19, further including a control feature selected from a tag cassette, a transduction marker, and/or a suicide switch.

21. A genetic construct encoding a CAR of any of embodiments 1-20.

22. A genetic construct of embodiment 21, wherein the genetic construct includes the 1H10-intDS-41bb-3z-T-CD19t Top Strand as set forth in SEQ ID NO: 46;
1H10-sh-41bb-3z-T-CD19t Top Strand as set forth in SEQ ID NO: 47;
1A9-intDS-41bb-3z-T-CD19t Top Strand as set forth in SEQ ID NO: 48;
1A9-sh-41bb-3z-T-CD19t Top Strand as set forth in SEQ ID NO: 49;
1E6-intDS-41bb-3z-T-CD19t Top Strand as set forth in SEQ ID NO: 50;
1E6-sh-41bb-3z-T-CD19t Top Strand as set forth in SEQ ID NO: 51;
1H10-LvHv-intDS-41bb-3z-T-CD19t Top Strand as set forth in SEQ ID NO: 200;
1A9-LvHv-intDS-41bb-3z-T-CD19t Top Strand as set forth in SEQ ID NO: 201;
1E6-LvHv-intDS-41bb-3z-T-CD19t Top Strand as set forth in SEQ ID NO: 202; or
2D3-LvHv-intDS-41bb-3z-T-CD19t Top Strand as set forth in SEQ ID NO: 203.

23. A nanoparticle encapsulating the genetic construct of embodiments 21 or 22.

24. A cell genetically modified to express the CAR of any of embodiments 1-20 and/or including a genetic construct of embodiments 21 or 22.

25. A cell of embodiment 24, wherein the cell is an autologous cell or an allogeneic cell in reference to a subject.

26. A cell of embodiments 24 or 25, wherein the cell is in vivo or ex vivo.

27. A cell of any of embodiments 24-26, wherein the cell is a T cell, B cell, natural killer (NK) cell, NK-T cell, monocyte/macrophage, hematopoietic stem cells (HSC), or a hematopoietic progenitor cell (HPC).

28. A cell of any of embodiments 24-27, wherein the cell is a T cell selected from a CD3+ T cell, a CD4+ T cell, a CD8+ T cell, a central memory T cell, an effector memory T cell, and/or a naive T cell.

29. A cell of any of embodiments 24-28, wherein the cell is a CD8+ T cell.

30. A cell of any of embodiments 24-29, wherein the cell has been incubated in a cell media including IL-2, IL-7, IL-15, and/or IL-21.

31. A cell embodiment 30, wherein the cell has been incubated in a cell media including IL-2.

32. A cell of embodiment 31, wherein the cell media includes 10-100 ng/mL IL-2.

33. A cell of embodiments 31 or 32, wherein the cell media includes 50 ng/mL IL-2.

34. A cell of embodiment 30, wherein the cell has been incubated in a cell media including IL-7 and IL-15.

35. A cell of embodiment 34, wherein the cell media includes 5-15 ng/mL IL-7 and 5-15 ng/mL IL-15.

36. A cell of embodiments 34 or 35, wherein the cell media includes 10 ng/mL IL-7 and 10 ng/mL IL-15.

37. A cell of embodiment 30, wherein the cell media includes IL-7, IL-15, and IL-21.

38. A cell of embodiment 37, wherein the cell media includes 5-15 ng/mL IL-7, 5-15 ng/mL IL-15, and 5-15 ng/mL IL-21.

39. A cell of embodiments 37 or 38, wherein the cell media includes 10 ng/mL IL-7, 10 ng/mL IL-15, and 10 ng/mL IL-21.

40. A population of cells of any of embodiments 24-39 formulated for administration to a subject.

41. A method of treating a subject with a CD33-related disorder including administering a therapeutically effective amount of the nanoparticle of embodiment 23 or the population of cells of embodiment 40 to the subject thereby treating the subject with the CD33-related disorder.

42. A method of embodiment 41, wherein the cell population includes autologous cells or allogeneic cells.

43. A method of embodiments 41 or 42, wherein the CD33-related disorder includes acute myeloid leukemia (AML).

44. A method of embodiments 41 or 42, wherein the CD33-related disorder includes acute lymphoblastic leukemia (ALL), chronic myelogenous leukemia (CML), chronic myelomonocytic leukemia (CML), mast cell leukemia, myelodysplastic syndrome (MDS), B-cell acute lymphoblastic leukemia (B-ALL), T-cell acute lymphoblastic leukemia (T-ALL), or megakaryocytic leukemia.

45. A method of any of embodiments 41-44, further including determining whether the subject expresses or lacks the V-set domain of CD33, and
if the subject expresses the V-set domain of CD33, selecting a combination therapy including a composition encoding a binding domain of one or more of 1H10, 1A9, 1E6, 1D2, and 1B9 and a binding domain of one or more of one or more of 1H8, 2D3, and 2E3.

46. A method of any of embodiments 41-44, further including determining whether the subject expresses or lacks the V-set domain of CD33, and
if the subject does not express the V-set domain of CD33, selecting a therapy including a composition encoding a binding domain of one or more of 6H9, 9G2, 3A5, 7D5, 1H7, and 2D5.

47. A method of activating an immune response against CD33-expressing cells in a subject in need thereof including administering a therapeutically effective amount of the nanoparticle of embodiment 23 or the population of cells of embodiment 40 to the subject activating an immune response against CD33-expressing cells in the subject in need.

48. A method of embodiment 47, wherein the cell population includes autologous cells or allogeneic cells.

49. A method of embodiments 47 or 48, wherein the CD33-expressing cells include acute myeloid leukemia (AML) cells.

50. A method of embodiments 47 or 48, wherein the CD33-expressing cells include acute lymphoblastic leukemia (ALL), chronic myelogenous leukemia (CML), chronic myelomonocytic leukemia (CML), mast cell leukemia, myelodysplastic syndrome (MDS), B-cell acute lymphoblastic leukemia (B-ALL), T-cell acute lymphoblastic leukemia (T-ALL), or megakaryocytic leukemia.

51. A method of any of embodiments 47-50, wherein the cell population includes autologous cells or allogeneic cells.

52. A method of any of embodiments 47-51, further including determining whether the subject expresses or lacks the V-set domain of CD33, and
if the subject expresses the V-set domain of CD33, selecting a combination therapy including a composition encoding a binding domain of one or more of 1H10, 1A9, 1E6, 1D2, and 1B9 and a binding domain of one or more of one or more of 1H8, 2D3, and 2E3.

53. A method of any of embodiments 47-51, further including determining whether the subject expresses or lacks the V-set domain of CD33, and
if the subject does not express the V-set domain of CD33, selecting a therapy including a composition encoding a binding domain of one or more of 6H9, 9G2, 3A5, 7D5, 1 H7, and 2D5.

54. A kit including a nucleotide sequence encoding a CAR including a binding domain of one or more of 1H10, 1A9, 1E6, 1D2, and 1B9 and a nucleotide sequence encoding a CAR including a binding domain of one or more of one or more of 1H8, 2D3, and 2E3.

55. A kit including a nucleotide sequence encoding a CAR including a binding domain of one or more of 1H10, 1A9, 1E6, 1D2, and 1B9.

### (IX) CLOSING PARAGRAPHS

The nucleic acid and amino acid sequences provided herein are shown using letter abbreviations for nucleotide bases and amino acid residues, as defined in 37 C.F.R. § 1.822 and set forth in the tables in WIPO Standard ST.25 (1998), Appendix 2, Tables 1 and 3. Only one strand of each nucleic acid sequence is shown, but the complementary strand is understood as included in embodiments where it would be appropriate.

To the extent not explicitly provided herein, coding sequences for proteins disclosed herein and protein sequences for coding sequences disclosed herein can be readily derived from one of ordinary skill in the art.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (nonpolar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol.

215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, a CAR binding domain or a nanoparticle selected cell targeting ligand) to its cognate binding molecule with an affinity or Kₐ (i.e., an equilibrium association constant of a particular binding interaction with units of 1/M) equal to or greater than 10⁵ M⁻¹, while not significantly associating with any other molecules or components in a relevant environment sample. Binding domains may be classified as “high affinity” or “low affinity”. In particular embodiments, “high affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Kₐ (equilibrium association constant) for the cognate binding molecule that is higher than the reference binding domain or due to a K_{d}(dissociation constant) for the cognate binding molecule that is less than that of the reference binding domain, or due to an off-rate (K_{off}) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in CD33-expressing cell lysis in an vitro assay cell killing assay, as described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 19% of the stated value; ±18% of the stated value; 17% of the stated value; 16% of the stated value; ±15% of the stated value; 14% of the stated value; ±13% of the stated value; 12% of the stated value; 11% of the stated value; 10% of the stated value; 9% of the stated value; 8% of the stated value; 7% of the stated value; ±6% of the stated value; 5% of the stated value; 4% of the stated value; ±3% of the stated value; 2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A chimeric antigen receptor (CAR) comprising
an extracellular component comprising a binding domain having a complementarity determining region (CDR) set of antibody 1E6, 1H10, 1A9, 1D2, 1B9, 1H8, 2D3, or 2E3, according to North, IMGT, Kabat or Chothia;
an intracellular component comprising an effector domain; and
a transmembrane domain linking the extracellular component to the intracellular component.

2. The CAR of claim 1, wherein the binding domain comprises a single chain variable fragment (scFv).

3. The CAR of claim 2, wherein the scFv is encoded by
the 1H10 scFv coding sequence as set forth in SEQ ID NO: 2;
the 1A9 scFv coding sequence as set forth in SEQ ID NO: 3;
the 1E6 scFv coding sequence as set forth in SEQ ID NO: 4;
the 1 D2 scFv coding sequence as set forth in SEQ ID NO: 5;
the 1B9 scFv coding sequence as set forth in SEQ ID NO: 6;
the 1H8 scFv coding sequence as set forth in SEQ ID NO: 7;
the 2D3 scFv coding sequence as set forth in SEQ ID NO: 8; or
the 2E3 scFv coding sequence as set forth in SEQ ID NO: 9.

4. The CAR of claim 2, wherein the scFv is
the 1H10 scFv in the VH-VL orientation as set forth in SEQ ID NO: 190;
the 1H10 scFv in the VL-VH orientation as set forth in SEQ ID NO: 191;
the 1A9 scFv in the VH-VL orientation as set forth in SEQ ID NO: 192;
the 1A9 scFv in the VL-VH orientation as set forth in SEQ ID NO: 193;
the 1E6 scFv in the VH-VL orientation as set forth in SEQ ID NO: 194;
the 1E6 scFv in the VL-VH orientation as set forth in SEQ ID NO: 195;
the 2D3 scFv in the VH-VL orientation as set forth in SEQ ID NO: 196; or
the 2D3 scFv in the VL-VH orientation as set forth in SEQ ID NO: 197.

5. The CAR of claim 1, wherein the extracellular component further comprises a spacer region.

6. The CAR of claim 5, wherein the spacer region is 135 amino acids or less or 16 amino acids of less.

7. The CAR of claim 5, wherein the spacer region is 131 amino acids or less and consists of the hinge region and CH3 domain of IgG4.

8. The CAR of claim 5, wherein the spacer region is 12 amino acids or less and consists of the hinge region of IgG4.

9. The CAR of claim 7, wherein the IgG4 is human IgG4.

10. The CAR of claim 5, wherein the spacer region is encoded by the sequence as set forth in SEQ ID NO: 10; SEQ ID NO: 11; or SEQ ID NO: 12.

11. The CAR of claim 1, wherein the effector domain comprises all or a portion of the signaling domain of CD3ζ; all or a portion of the signaling domain of 4-1BB, all or a portion of the signaling domain of CD28, all or a portion of the signaling domain of CD3ζ and 4-1BE; all or a portion of the signaling domain of CD3ζ and CD28; or all or a portion of the signaling domain of CD3ζ, 4-1 BB, and CD28

12. The CAR of claim 1, wherein the effector domain comprises all or a portion of the signaling domain of CD3ζ and 4-11BE.

13. The CAR of claim 11, wherein the CD3ζ signaling domain is encoded by the CD3ζ coding sequence as set forth in SEQ ID NO: 14.

14. The CAR of claim 11, wherein the CD3ζ has the sequence as set forth in SEQ ID NO: 15 or SEQ ID NO: 16.

15. The CAR of claim 11, wherein the 4-1 BB signaling domain is encoded by the sequence as set forth in SEQ ID NO: 17 or SEQ ID NO: 18.

16. The CAR of claim 11, wherein the 4-1BB has the sequence as set forth in SEQ ID NO: 19 or SEQ ID NO: 20.

17. The CAR of claim 1, wherein the transmembrane domain comprises a CD28 transmembrane domain.

18. The CAR of claim 17, wherein the CD28 transmembrane domain is encoded by the sequence as set forth in SEQ ID NO: 21; SEQ ID NO: 22; or SEQ ID NO: 23.

19. The CAR of claim 17, wherein the CD28 transmembrane domain has the sequence as set forth in SEQ ID NO: 24 or SEQ ID NO: 25.

20. The CAR of claim 1, further comprising a control feature selected from a tag cassette, a transduction marker, and/or a suicide switch.

21. A genetic construct encoding a CAR of claim 1.

22. The genetic construct of claim 21, wherein the genetic construct comprises the sequence as set forth in SEQ ID NO: 46; SEQ ID NO: 47; SEQ ID NO: 48; SEQ ID NO: 49; SEQ ID NO: 50; SEQ ID NO: 51; SEQ ID NO: 200; SEQ ID NO: 201; SEQ ID NO: 202; or SEQ ID NO: 203.

23. A nanoparticle encapsulating a genetic construct of claim 21 or 22.

24. A cell genetically modified to express a CAR of claim 1.

25. The cell of claim 24, wherein the cell is an autologous cell or an allogeneic cell in reference to a subject.

26. The cell of claim 24, wherein the cell is in vivo or ex vivo.

27. The cell of claim 24, wherein the cell is a T cell, B cell, natural killer (NK) cell, NK-T cell, monocyte/macrophage, hematopoietic stem cells (HSC), or a hematopoietic progenitor cell (HPC).

28. The cell of claim 24, wherein the cell is a T cell selected from a CD3+ T cell, a CD4+ T cell, a CD8+ T cell, a central memory T cell, an effector memory T cell, and/or a naïve T cell.

29. The cell of claim 24, wherein the cell is a CD8+ T cell.

30. The cell of claim 24, wherein the cell has been incubated in a cell media comprising IL-2, IL-7, IL-15, and/or IL-21.

31. The cell of claim 30, wherein the cell has been incubated in a cell media comprising IL-2.

32. The cell of claim 31, wherein the cell media comprises 10-100 ng/mL IL-2.

33. The cell of claim 32, wherein the cell media comprises 50 ng/mL IL-2.

34. The cell of claim 30, wherein the cell has been incubated in a cell media comprising IL-7 and IL-15.

35. The cell of claim 34, wherein the cell media comprises 5-15 ng/mL IL-7 and 5-15 ng/mL IL-15.

36. The cell of claim 35, wherein the cell media comprises 10 ng/mL IL-7 and 10 ng/mL IL-15.

37. The cell of claim 30, wherein the cell media comprises IL-7, IL-15, and IL-21.

38. The cell of claim 37, wherein the cell media comprises 5-15 ng/mL IL-7, 5-15 ng/mL IL-15, and 5-15 ng/mL IL-21.

39. The cell of claim 38, wherein the cell media comprises 10 ng/mL IL-7, 10 ng/mL IL-15, and 10 ng/mL IL-21.

40. A population of cells of claim 24 formulated for administration to a subject.

41. A method of treating a subject with a CD33-related disorder comprising administering a therapeutically effective amount of the nanoparticle of claim 23 or the population of cells of claim 40 to the subject thereby treating the subject with the CD33-related disorder.

42. The method of claim 41, wherein the population of cells comprise autologous cells or allogeneic cells.

43. The method of claim 41, wherein the CD33-expressing cells comprise acute myeloid leukemia (AML) cells.

44. The method of claim 41, wherein the CD33-related disorder comprises acute Page 3 of 5 lymphoblastic leukemia (ALL), chronic myelogenous leukemia (CML), chronic myelomonocytic leukemia (CML), mast cell leukemia, myelodysplastic syndrome (MDS), B-cell acute lymphoblastic leukemia (B-ALL), T-cell acute lymphoblastic leukemia (T-ALL), or megakaryocytic leukemia.

45. The method of claim 41, further comprising determining whether the subject expresses or lacks the V-set domain of CD33, and
if the subject expresses the V-set domain of CD33, selecting a combination therapy comprising a composition comprising a binding domain of one or more of 1H10, 1A9, 1E6, 1D2, and 1B9 and
a binding domain of one or more of one or more of 1H8, 2D3, and 2E3.

46. The method of claim 41, further comprising determining whether the subject expresses or lacks the V-set domain of CD33, and
if the subject does not express the V-set domain of CD33, selecting a therapy comprising a composition comprising a binding domain of one or more of 1H10, 1A9, 1E6, 1D2, and 1B9.

47. A method of activating an immune response against CD33-expressing cells in a subject in need thereof comprising administering a therapeutically effective amount of the nanoparticle of claim 23 or the population of cells of claim 40 to the subject activating an immune response against CD33-expressing cells in the subject in need.

48. The method of claim 47, wherein the cell population comprises autologous cells or allogeneic cells.

49. The method of claim 47, wherein the CD33-expressing cells comprise acute myeloid leukemia (AML) cells.

50. The method of claim 47, wherein the CD33-expressing cells comprise acute lymphoblastic leukemia (ALL), chronic myelogenous leukemia (CML), chronic myelomonocytic leukemia (CML), mast cell leukemia, myelodysplastic syndrome (MDS), B-cell acute lymphoblastic leukemia (B-ALL), T-cell acute lymphoblastic leukemia (T-ALL), or megakaryocytic leukemia.

51. The method of claim 47, further comprising determining whether the subject expresses or lacks the V-set domain of CD33, and
if the subject expresses the V-set domain of CD33, selecting a combination therapy comprising a composition comprising a binding domain of one or more of 1H10, 1A9, 1E6, 1D2, and 1B9 and
a binding domain of one or more of one or more of 1H8, 2D3, and 2E3.

52. The method of claim 47, further comprising determining whether the subject expresses or lacks the V-set domain of CD33, and
if the subject does not express the V-set domain of CD33, selecting a therapy comprising a composition comprising a binding domain of one or more of 1H10, 1A9, 1E6, 1D2, and 1B9.

53. A kit comprising a nucleotide sequence encoding a CAR comprising a binding domain of one or more of 1H10, 1A9, 1E6, 1D2, and 1B9 and a nucleotide sequence encoding a CAR comprising a binding domain of one or more of one or more of 1H8, 2D3, and 2E3.
